(19) United States
(12) Patent Application Publication
(10) Pub. No.: US 2016/0052936 A1
(43) Pub. Date: Feb. 25, 2016

(54) SPIROINDOLINE DERIVATIVES FOR USE AS GONADOTROPIN-RELEASING HORMONE RECEPTOR ANTAGONISTS

(71) Applicant: BAYER PHARMA AKTIENGESELLSCHAFT, Berlin (DE)

(72) Inventors: Olaf PANKNIN, Berlin (DE); Sven RING, Jena (DE); Stefan BÄURLE, Berlin (DE); Andrea WAGENFELD, Berlin (DE); Reinhard NUBBEMEYER, Berlin (DE); Katrin NOWAK-REPPEL, Berlin (DE); Gernot LANGER, Falkensee (DE)

(73) Assignee: BAYER PHARMA AKTIENGESELLSCHAFT, Berlin (DE)

(21) Appl. No.: 14/783,458

(22) PCT Filed: Apr. 8, 2014
(86) PCT No.: PCT/EP2014/057079

§ 371 Date: Oct. 9, 2015

(30) Foreign Application Priority Data
Apr. 9, 2013 (EP) 13162986.7

(51) Int. Cl. C07D 495/10

(52) U.S. Cl. CPC C07D 495/10

(57) ABSTRACT

Spiroindoline derivatives, processes for their preparation and pharmaceutical compositions thereof, their use for the treatment of diseases, and their use for the manufacture of medicaments for the treatment of diseases, especially sex-hormone-related diseases in both men and women, in particularly those selected from the group of endometriosis, uterine leiomyoma (fibroids), polycystic ovarian disease, menorrhagia, dysmenorrhea, hirsutism, precocious puberty, gonadal steroid-dependent neoplasia such as cancers of the prostate, breast and ovary, gonadotrope pituitary adenomas, sleep apnea, irritable bowel syndrome, premenstrual syndrome, benign prostatic hypertrophy, contraception, infertility and assisted reproductive therapy such as in vitro fertilization. The present application relates in particular to spiroindoline derivatives as gonadotropin-releasing hormone (GnRH) receptor antagonists.

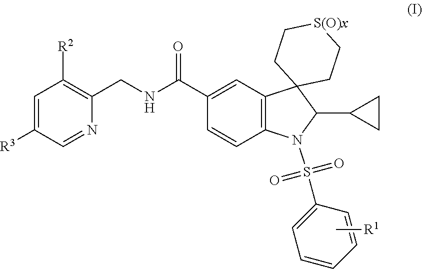

### TECHNICAL FIELD

The present invention refers to spiroindoline derivatives as gonadotropin-releasing hormone (GnRH) receptor antagonists according to Formula (I)

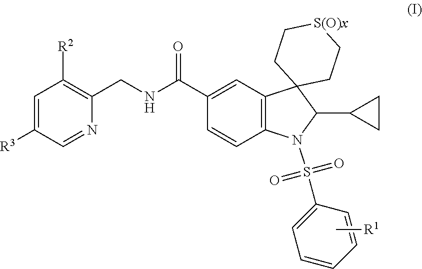

in which
x=0, 1 or 2;
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN;
   with the proviso of N-[(3-chloro-5-fluoropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide, pharmaceutical compositions containing a spiroindoline derivative according to Formula (I) and methods of treating disorders by administration of a spiroindoline derivative according to Formula (I) to a mammal, particularly a human, in need thereof.

### BACKGROUND ART

Gonadotropin-releasing hormone (GnRH) is a decapeptide (pGlu-His-Trp-Ser-Tyr-Gly-Leu-Arg-Pro-Gly-NH₂) released from the hypothalamus, also known as luteinizing hormone-releasing hormone (LHRH). GnRH acts on the pituitary gland to stimulate the biosynthesis and release of luteinizing hormone (LH) and follicle-stimulating hormone (FSH). LH released from the pituitary gland is responsible for the regulation of gonadal steroid production in both genders and late ovarian follicle development and ovulation in female mammals, FSH regulates spermatogenesis in males and early follicular development in females. Thus GnRH plays a key role in human reproduction.

As a consequence of its biological significance, synthetic antagonists and agonists to GnRH have been the center of several research activities, particularly in the field of endometriosis, uterine leiomyoma (fibroids), prostate cancer, breast cancer, ovarian cancer, prostatic hyperplasia, assisted reproductive therapy and precocious puberty.

For example, peptidic GnRH agonists, such as leuprorelin (pGlu-His-Trp-Ser-Tyr-d-Leu-Leu-Arg-Pro-NHEt), are described for the use in the treatment of such conditions (The Lancet 2001, 358, 1793-1803; Mol. Cell. Endo. 2000, 166, 9-14). Said agonists initially induce the synthesis and release of gonadotropins, by binding to the GnRH receptor on the pituitary gonadotrophic cells (‘flare-up’). However, chronic administration of GnRH agonists reduces gonadotropin release from the pituitary and results in the down-regulation of the receptor, with the consequence of suppressing sex steroidal hormone production after some period of treatment.

GnRH antagonists, on the contrary, are supposed to suppress gonadotropins from the onset, offering several advantages, in particular a lack of side effects associated with the flare up seen under GnRH superagonist treatment. Several peptidic antagonists with low histamine release potential are known in the art. Said peptidic products show low oral bioavailability which limits their clinical use.

Nonpeptidic compounds active as GnRH receptor antagonists are described in WO2011/076687, WO05/007165, WO03/064429 and WO04/067535. Although intensive research has been driven for more than 15 years aiming at non-peptidic GnRH antagonists, none of them succeeded so far to reach the market.

Nevertheless, effective small molecule GnRH receptor ligands, especially compounds which are active as antagonists as well as pharmaceutical compositions containing such GnRH receptor antagonists and methods relating to the use thereof to treat, for example, sex-hormone-related conditions, in particular for the treatment of leiomyoma are still highly required in the pharmaceutical field.

The spiroindoline derivatives according to the present invention aim to fulfill such unmet need, and provide at the same time further advantages over the known art.

Spiroindoline derivatives are known in the art as pharmaceutically active ingredients and in the cropscience field as insecticides.

The document WO00/66554 describes generic indolines as potential progesteron receptor antagonists.

The document US2006/63791 refers to compounds useful in the treatment of tumors and cancers and, on page 20, describes the synthesis of a nitroindoline by condensing an aldehyde and a phenylhydrazine under acidic conditions (Fischer indole synthesis) and subsequent reduction of the indolenine intermediate.

The document WO13/017678 refers to compounds useful for the treatment of helminth infections and parasitoses in animals. Said document, pp. 38-39, describes the synthesis of a spiroindoline-piperidine via Fischer indole synthesis by condensing an aldehyde or an enol ether and a phenylhydrazine under acidic conditions and subsequent reduction of the indolenine intermediate.

Liu et al. describes the synthesis of a spiroindoline-tetrahydropyrane in a similar manner in a one-pot reaction (Tetrahedron 2010, 66, 3, 573-577).

The document WO10/151737 refers to compounds useful for the treatment of general inflammation and, on page 224, describes the synthesis of an indolenine mixture in an analogous Fischer indole synthesis by condensing an aldehyde with a phenylhydrazine.

The document WO06/090261 refers to compounds useful for treating abnormal cell growth in mammal, and describes on pp. 67-68 the synthesis of a spiroindoline-piperidine via Fischer indole synthesis and subsequent addition of a Grignard reagent to the indolenine intermediate.

The document WO08/157741 discloses compounds useful in the treatment of diseases associated with the overexpression of CCR2, and describes on pp. 41-42 the synthesis of a spiroindoline-piperidine starting from an oxindole precursor via Grignard addition and subsequent deoxygenation.

The document WO93/15051 discloses a generic oxindole as potential vasopressin/oxytocin antagonists.

Further spiroindoline derivatives with pharmaceutical properties were disclosed for example in the documents WO1994/29309, WO1999/64002 and WO2002/47679.

Spiroindoline derivatives active as GnRH receptor antagonists have been described for the first time in EP2013/050676.

### DISCLOSURE OF THE INVENTION

The aim of the present invention is to provide gonadotropin-releasing hormone (GnRH) receptor antagonists, methods for their preparation, and pharmaceutical compositions containing the same as well as the use of the same for the treatment of endometriosis, uterine leiomyoma (fibroids), polycystic ovarian disease, menorrhagia, dysmenorrhea, hirsutism, precocious puberty, gonadal steroid-dependent neoplasia such as cancers of the prostate, breast and ovary, gonadotrope pituitary adenomas, sleep apnea, irritable bowel syndrome, premenstrual syndrome, benign prostatic hypertrophy, contraception, infertility, assisted reproductive therapy such as in vitro fertilization, in the treatment of growth hormone deficiency and short stature, and in the treatment of systemic lupus erythematosus, and in particular in the treatment of endometriosis, uterine leiomyoma (fibroids), prostate cancer, breast cancer, ovarian cancer, benign prostatic hypertrophy, assisted reproductive therapy, menorrhagia, dysmenorrhea and precocious puberty.

In particular, the present invention relates to compounds according to Formula (I)

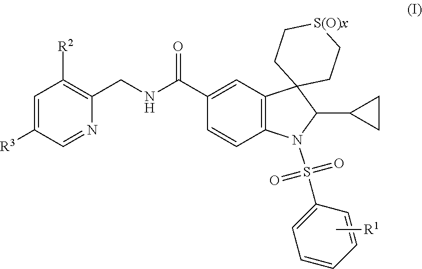

in which
x=0, 1 or 2;
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN;
   with the proviso of N-[(3-chloro-5-fluoropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide.

A particular form of the invention refers to the compounds according to Formula (Ia)

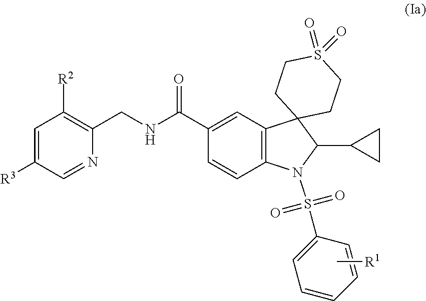

in which
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN;
   with the proviso of N-[(3-chloro-5-fluoropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide.

A further particular form of the invention refers to the compounds according to Formula (Ib)

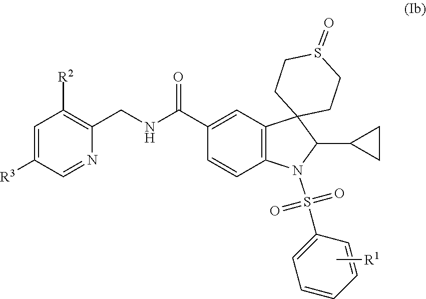

in which
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN.

A further particular form of the invention refers to the compounds according to Formula (Ic)

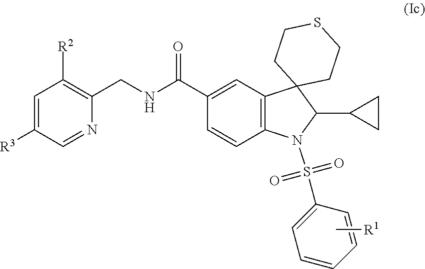

in which
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN.

Compounds according to the invention are the compounds of the formulae (I), (Ia), (Ib), (Ic) and the salts, solvates and solvates of the salts thereof, the compounds which are encompassed by formulae (I), (Ia), (Ib), (Ic) and are of the formulae mentioned hereinafter, and the salts, solvates and solvates of the salts thereof, and the compounds which are encompassed by formulae (I), (Ia), (Ib), (Ic) and are mentioned hereinafter as exemplary embodiments, and the salts, solvates and solvates of the salts thereof, insofar as the compounds encompassed by formulae (I), (Ia), (Ib), (Ic) and mentioned hereinafter are not already salts, solvates and solvates of the salts.

Hydrates of the compounds of the invention or their salts are stoichiometric compositions of the compounds with water, such as, for example, hemi-, mono-, or dihydrates.

Solvates of the compounds of the invention or their salts are stoichiometric compositions of the compounds with solvents.

Solvates which are preferred for the purposes of the present invention are hydrates.

Salts for the purposes of the present invention are preferably pharmaceutically acceptable salts of the compounds according to the invention (for example, see S. M. Berge et al., “Pharmaceutical Salts”, J. Pharm. Sci. 1977, 66, 1-19).

Pharmaceutically acceptable salts include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, maleic, fumaric, benzoic, ascorbic, succinic, acetic, trifluoroacetic, oxalic, propionic, tartaric, salicylic, citric, gluconic, lactic, mandelic, cinnamic, aspartic, stearic, palmitic, glycolic, and glutamic acid.

Pharmaceutically acceptable salts also include salts of customary bases, such as for example and preferably alkali metal salts (for example sodium, lithium and potassium salts), alkaline earth metal salts (for example calcium and magnesium salts), and ammonium salts derived from ammonia or organic amines, such as illustratively and preferably ethylamine, diethylamine, triethylamine, ethyldiisopropylamine, monoethanolamine, diethanolamine, triethanolamine, dicyclohexylamine, dimethylaminoethanol, benzylamine, dibenzylamine, N-methylmorpholine, N-methylpiperidine, dihydroabietyl-amine, arginine, lysine, and ethylenediamine.

Also encompassed are salts which are themselves unsuitable for pharmaceutical uses but can be used for example for isolating or purifying the compounds of the invention.

The present invention additionally encompasses prodrugs of the compounds of the invention. The term “prodrugs” encompasses compounds which themselves may be biologically active or inactive, but are converted during their residence time in the body into compounds of the invention (for example by metabolism or hydrolysis).

The present invention includes all possible stereoisomers of the compounds of the present invention as single stereoisomers, or as any mixture of said stereoisomers, e.g. R- or S-isomers, or E- or Z-isomers, in any ratio.

All isomers, whether separated, pure, partially pure, or in racemic mixture, of the compounds of this invention are encompassed within the scope of this invention. The purification of said isomers and the separation of said isomeric mixtures may be accomplished by standard techniques known in the art. For example, diastereomeric mixtures can be separated into the individual isomers by chromatographic processes or crystallization, and racemates can be separated into the respective enantiomers either by chromatographic processes on chiral phases or by resolution.

If the compounds of the invention may occur in tautomeric forms, the present invention encompasses all tautomeric forms.

Unless otherwise stated, the following definitions apply for the substituents and residues used throughout this specification and claims. The particularly named chemical groups and atoms (for example fluorine, methyl, methyloxy and so on) should be considered as particular forms of embodiment for the respective groups in compounds according to the invention.

The term “halogen atom” or “halo” is to be understood as meaning a fluorine, chlorine, bromine or iodine atom, most preferably fluorine.

The term “C₁-C₄-alkyl” is to be understood as preferably meaning a linear or branched, saturated, monovalent hydrocarbon group having 1, 2, 3, or 4 carbon atoms, e.g. a methyl, ethyl, propyl, butyl, isopropyl, isobutyl, sec-butyl or tert-butyl group, or an isomer thereof. Particularly, said group has 1, 2 or 3 carbon atoms (“C₁-C₃-alkyl”), methyl, ethyl, n-propyl- or iso-propyl.

The term “halo-C₁-C₄-alkyl” is to be understood as preferably meaning a linear or branched, saturated, monovalent hydrocarbon group in which the term “C₁-C₄-alkyl” is defined supra, and in which one or more hydrogen atoms is replaced by a halogen atom, in the same way or differently, i.e. one halogen atom being independent from another.

Particularly, said halogen atom is a fluorine atom. Said halo-C₁-C₄-alkyl group is in particular —CF₃, —CHF₂, —CH₂F, —CF₂CF₃, —CF₂CH₃, or —CH₂CF₃.

The term “C₁-C₄-alkoxy” is to be understood as preferably meaning a linear or branched, saturated, monovalent, hydrocarbon group of formula —O-alkyl, in which the term “alkyl” is defined supra, e.g. a methoxy, ethoxy, propoxy, isopropoxy, butoxy, isobutoxy, tert-butoxy or sec-butoxy group, or an isomer thereof.

The term “halo-C₁-C₄-alkoxy” is to be understood as preferably meaning a linear or branched, saturated, monovalent C₁-C₄-alkoxy group, as defined supra, in which one or more of the hydrogen atoms is replaced, in the same way or differently, by a halogen atom. Particularly, said halogen atom is a fluorine atom. Said halo-C₁-C₄-alkoxy group is, for example, —OCF₃, —OCHF₂, —OCH₂F, —OCF₂CF₃, or —OCH₂CF₃.

The term “C₁-C₄”, as used throughout this text, e.g. in the context of the definition of “C₁-C₄-alkyl”, “C₁-C₄-haloalkyl”, “C₁-C₄-alkoxy”, or “C₁-C₄-haloalkoxy” is to be understood as meaning an alkyl group having a finite number of carbon atoms of 1 to 4, i.e. 1, 2, 3 or 4 carbon atoms. It is to be understood further that said term “C₁-C₄” is to be interpreted as any sub-range comprised therein, e.g. C₁-C₄, C₂-C₃, C₁-C₂, C₁-C₃; particularly C₁-C₂, C₁-C₃, C₁-C₄; more particularly C₁-C₄; in the case of “C₁-C₄-haloalkyl” or “C₁-C₄-haloalkoxy” even more particularly C₁-C₂.

Oxo represents a double-bonded oxygen atom.

As used herein, the term “one or more times”, e.g. in the definition of the substituents of the compounds of the general formulae of the present invention, is understood as meaning “one, two, three, four or five times”, particularly “one, two, three or four times”, more particularly “one, two or three times”, even more particularly “one or two times”.

Throughout this document, for the sake of simplicity, the use of singular language is given preference over plural language, but is generally meant to include the plural language if not otherwise stated. E.g., the expression “A method of treating a disease in a patient, comprising administering to a patient an effective amount of a compound of formula (I)” is meant to include the simultaneous treatment of more than one disease as well as the administration of more than one compound of formula (I).

Particular forms of embodiment of compounds of the general formula (I) as described above are going to be illustrated in the following.

In conjunction with the above or below definitions and embodiments, compounds according to formulae (I), (Ia), (Ib) and (Ic) are in particular those in which R¹ is a single group in para or meta position and is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂, according to a further particular embodiment R¹ is a single group in para or meta position selected from the group consisting of halogen, C₁-C₄-alkoxy, halo-C₁-C₄-alkoxy, CN, C(O)NH₂, more particularly R¹ is a single group in para position selected from the group consisting of F, Cl, OCF₂H, CN, C(O)NH₂, or a single group in meta position selected from the group consisting of OCH₃, OCF₂H, OCF₃, CN.

Compounds according to formulae (I), (Ia), (Ib) and (Ic) according to the present invention further comprise as a particular form of embodiment R² being selected from the group consisting of halogen, halo-C₁-C₄-alkyl, and more particularly R² being selected from the group consisting of F, Cl, CF₃.

A further particular form of embodiment according to the invention refers to compounds according to formulae (I), (Ia), (Ib) and (Ic) in which R³ is selected from the group consisting of halogen, C₁-C₄-alkyl, halo-C₁-C₄-alkyl, more particularly R³ is selected from the group consisting of Cl, CH₃, CF₃.

Compounds of formulae (I), (Ia), (Ib) and (Ic) according to the invention are in particular those in which R¹ is a single group in para or meta position and is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂, R² is selected from the group consisting of F, Cl, CF₃, and R³ is selected from the group consisting of Cl, CH₃, CF₃, and more particularly in that R² is a Cl, and R³ is a CF₃.

Furthermore, compounds of formulae (I), (Ia), (Ib) and (Ic) according to the invention are in particular those in which R¹ is a single group in para or meta position selected from the group consisting of halogen, C₁-C₄-alkoxy, halo-C₁-C₄-alkoxy, CN, C(O)NH₂, R² is selected from the group consisting of F, Cl, CF₃, and R³ is selected from the group consisting of Cl, CH₃, CF₃, and more particularly in that R² is a Cl, and R³ is a CF₃.

More particularly, compounds of formulae (I), (Ia), (Ib) and (Ic) according to the invention are those in which R¹ a single group in para position selected from the group consisting of F, Cl, OCF₂H, CN, C(O)NH₂, R² is selected from the group consisting of F, Cl, CF₃, and R³ is selected from the group consisting of Cl, CH₃, CF₃, and more particularly in that R² is a Cl, and R³ is a CF₃.

Further compounds of formulae (I), (Ia), (Ib) and (Ic) according to the invention are those in which R¹ is a single group in meta position selected from the group consisting of OCH₃, OCF₂H, OCF₃, CN, R² is selected from the group consisting of F, Cl, CF₃, and R³ is selected from the group consisting of Cl, CH₃, CF₃, and more particularly in that R² is a Cl, and R³ is a CF₃.

Compounds according to the invention are:
2-cyclopropyl-1-[(4-fluorophenyl)sulfonyl]-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide
N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′-oxide
N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
N-{[5-chloro-3-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-{[5-methyl-3-(trifluoromethyl)pyridin-2-yl]methyl}-1,2,2′,3′,5′,6′-hexahydrospiro[indole-3,4′-thiopyran]-5-carboxamide 1′,1′-dioxide
2-cyclopropyl-N-{[3-fluoro-5-(trifluoromethyl)pyridin-2-yl]methyl}-1-[(3-methoxyphenyl)sulfonyl]-1,2,2′,3′,5′,6′-hexahydrospiro[indole-3,4′-thiopyran]-5-carboxamide 1′,1′-dioxide
1-[(4-cyanophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide
N-{[3-chloro-5-(trifluoromethyl)pyridin-2-yl]methyl}-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide
1-[(3-cyanophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide
N-{[3-chloro-5-(trifluoromethyl)pyridin-2-yl]methyl}-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide
2-cyclopropyl-N-{[3-fluoro-5-(trifluoromethyl)pyridin-2-yl]methyl}-1-{[3-(trifluoromethoxy)phenyl]sulfonyl}-1,2,2′,3′,5′,6′-hexahydrospiro[indole-3,4′-thiopyran]-5-carboxamide 1′,1′-dioxide
N-{[3-chloro-5-(trifluoromethyl)pyridin-2-yl]methyl}-2-cyclopropyl-1-{[3-(trifluoromethoxy)phenyl]sulfonyl}-1,2,2′,3′,5′,6′-hexahydrospiro[indole-3,4′-thiopyran]-5-carboxamide 1′,1′-dioxide
2-cyclopropyl-1-{[3-(difluoromethoxy)phenyl]sulfonyl}-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide
N-{[3-chloro-5-(trifluoromethyl)pyridin-2-yl]methyl}-2-cyclopropyl-1-{[3-(difluoromethoxy)phenyl]sulfonyl}-1,2,2′,3′,5′,6′-hexahydrospiro[indole-3,4′-thiopyran]-5-carboxamide 1′,1′-dioxide
2-cyclopropyl-1-{[4-(difluoromethoxy)phenyl]sulfonyl}-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide
N-{[3-chloro-5-(trifluoromethyl)pyridin-2-yl]methyl}-2-cyclopropyl-1-{[4-(difluoromethoxy)phenyl]sulfonyl}-1,2,2′,3′,5′,6′-hexahydrospiro[indole-3,4′-thiopyran]-5-carboxamide 1′,1′-dioxide
1-[(4-carbamoylphenyl)sulfonyl]-N-{[3-chloro-5-(trifluoromethyl)pyridin-2-yl]methyl}-2-cyclopropyl-1,2,2′,3′,5′,6′-hexahydrospiro[indole-3,4′-thiopyran]-5-carboxamide 1′,1′-dioxide
1-[(4-chlorophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide
1-[(4-chlorophenyl)sulfonyl]-N-{[3-chloro-5-(trifluoromethyl)pyridin-2-yl]methyl}-2-cyclopropyl-1,2,2′,3′,5′,6′-hexahydrospiro[indole-3,4′-thiopyran]-5-carboxamide 1′,1′-dioxide
N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide

Furthermore, compounds according to the invention are in particular those having chiral configuration S for the chiral carbon atom in position 2 of the 1,2,2′,3′,5′,6′-hexahydrospiro[indole-3,4′-thiopyran] ring on which the cyclopropyl group is bound ((2S)-2-cyclopropyl).

More particularly, compounds according to the invention are:
(2S)-2-cyclopropyl-1-[(4-fluorophenyl)sulfonyl]-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide
(2S)—N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
(2S)—N-{[3-chloro-5-(trifluoromethyl)pyridin-2-yl]methyl}-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide
(2S)—N-{[3-chloro-5-(trifluoromethyl)pyridin-2-yl]methyl}-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide

Another embodiment of the present invention provides compounds according to general formulae (I), (la), (Ib) and (Ic) and related specific embodiments for use as a medicament.

In another embodiment, the present invention provides a method of treating GnRH related disorder in a patient in need of such treatment, comprising administering to the patient an effective amount of a compound according to the invention as defined above.

In still another aspect, the invention provides use of a compound according to the invention as defined above for manufacturing a pharmaceutical composition for the treatment or prevention of GnRH related disorders.

The term “treating” or “treatment” as stated throughout this document is used conventionally, e.g., the management or care of a subject for the purpose of combating, alleviating, reducing, relieving, improving the condition of a disease or disorder, such as for example endometriosis and uterine leiomyoma (fibroids).

The term “subject” or “patient” includes organisms which are capable of suffering from a disorder or who could otherwise benefit from the administration of a compound of the invention, such as human and non-human animals. Preferred humans include human patients suffering from or prone to suffering from disorders, such as for example endometriosis and uterine fibroids. The term “non-human animals” includes vertebrates, e.g., mammals, such as non-human primates, sheep, cows, dogs, cats and rodents, e.g., mice, and non-mammals, such as chickens, amphibians, reptiles, etc.

In another aspect, the invention provides a pharmaceutical composition comprising a compound according to the invention, together with a pharmaceutically acceptable carrier.

In still another aspect, the invention provides a process for preparing a pharmaceutical composition. The process includes the step of combining at least one compound according to the invention as defined above with at least one pharmaceutically acceptable carrier, and bringing the resulting combination into a suitable administration form.

The compounds according to general formulae (I), (Ia), (Ib) and (Ic) are used as a medicament. In particular, said compounds are used to treat sexual hormone-related conditions in both men and women, as well as a mammal in general (also referred to herein as a “subject”). For example, such conditions include endometriosis, uterine leiomyoma (fibroids), polycystic ovarian disease, menorrhagia, dysmenorrhea, hirsutism, precocious puberty, gonadal steroid-dependent neoplasia such as cancers of the prostate, breast and ovary, gonadotrope pituitary adenomas, sleep apnea, irritable bowel syndrome, premenstrual syndrome, benign prostatic hypertrophy, contraception, infertility, assisted reproductive therapy such as in vitro fertilization, in the treatment of growth hormone deficiency and short stature, and in the treatment of systemic lupus erythematosus, and in particular in the treatment of endometriosis, uterine leiomyoma (fibroids), prostate cancer, breast cancer, ovarian cancer, benign prostatic hypertrophy, assisted reproductive therapy, menorrhagia, dysmenorrhea and precocious puberty.

The compounds according to general formulae (I), (Ia), (Ib) and (Ic) are further used as contraceptive.

The compounds of this invention are also useful as an adjunct to treatment of growth hormone deficiency and short stature, and for the treatment of systemic lupus erythematosus.

According to a further embodiment of the present invention the compounds according to general formulae (I), (Ia), (Ib) and (Ic) are also useful and can be used in combination with androgens, estrogens, progestins, SERMs, antiestrogens and antiprogestins for the treatment of endometriosis, uterine leiomyoma (fibroids), and in contraception, as well as in combination with an angiotensin-converting enzyme inhibitor, an angiotensin II-receptor antagonist, or a renin inhibitor for the treatment of uterine leiomyoma (fibroids). A combination of compounds according to general formulae (I), (Ia), (Ib) and (Ic) with bisphosphonates and other agents for the treatment and/or prevention of disturbances of calcium, phosphate and bone metabolism, and in combination with estrogens, SERMs, progestins and/or androgens for the prevention or treatment of bone loss or hypogonadal symptoms such as hot flushes during therapy with a GnRH antagonist is also part of the present invention.

The methods of this invention include administering an effective amount of a GnRH receptor antagonist, preferably in the form of a pharmaceutical composition, to a mammal in need thereof. Thus, in still a further embodiment, pharmaceutical compositions are disclosed containing one or more GnRH receptor antagonists of this invention in combination with a pharmaceutically acceptable carrier and/or diluent.

These and other aspects of the invention will be apparent upon reference to the following detailed description. To this end, various references are set forth herein which describe in more detail certain background information, procedures, compounds and/or compositions, and are each hereby incorporated by reference in their entirety.

The compounds of the present invention may generally be utilized as the free acid or free base. Alternatively, the compounds of this invention may be used in the form of acid or base addition salts.

Thus, the term “pharmaceutically acceptable salt” of compounds of general formulae (I), (Ia), (Ib) and (Ic) is intended to encompass any and all acceptable salt forms.

In addition, prodrugs are also included within the context of this invention. Prodrugs are any covalently bonded carriers that release a compound of general formulae (I), (Ia), (Ib) and (Ic) in vivo when such prodrug is administered to a patient. Prodrugs are generally prepared by modifying functional groups in a way such that the modification is cleaved, either by routine manipulation or in vivo, yielding the parent compound.

Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of general formulae (I), (Ia), (Ib) and (Ic). Further, in the case of a carboxylic acid (—COOH), esters may be employed, such as methyl esters, ethyl esters, and the like.

With regard to stereoisomers, the compounds of general formulae (I), (Ia), (Ib) and (Ic) may have chiral centers and may occur as racemates, racemic mixtures and as individual enantiomers or diastereomers. All such isomeric forms are included within the present invention, including mixtures thereof. Furthermore, some of the crystalline forms of the compounds of general formulae (I), (Ia), (Ib) and (Ic) may exist as polymorphs, which are included in the present invention. In addition, some of the compounds of general formulae (I), (Ia), (Ib) and (Ic) may also form solvates with water or other organic solvents. Such solvates are similarly included within the scope of this invention.

The effectiveness of a compound as a GnRH receptor antagonist may be determined by various assay techniques. Assay techniques well known in the field include the use of cultured pituitary cells for measuring GnRH activity (Vale et al., Endocrinology 1972, 91, 562-572) and the measurement of radioligand binding to rat pituitary membranes (Perrin et al., Mol. Pharmacol. 1983, 23, 44-51) or to membranes from cells expressing cloned receptors as described below. Other assay techniques include (but are not limited to) measurement of the effects of GnRH receptor antagonists on the inhibition of GnRH-stimulated calcium flux, modulation of phosphoinositol hydrolysis, and the circulating concentrations of gonadotropins in the castrate animal. Descriptions of these techniques, the synthesis of radiolabeled ligand, the employment of radiolabeled ligand in radioimmunoassay, and the measurement of the effectiveness of a compound as a GnRH receptor antagonist follow.

In another embodiment of the invention, pharmaceutical compositions containing one or more GnRH receptor antagonists are disclosed. For the purposes of administration, the compounds of the present invention may be formulated as pharmaceutical compositions.

Pharmaceutical compositions of the present invention comprise a GnRH receptor antagonist of the present invention and a pharmaceutically acceptable carrier and/or diluent. The GnRH receptor antagonist is present in the composition in an amount which is effective to treat a particular disorder that is, in an amount sufficient to achieve GnRH receptor antagonist activity, and preferably with acceptable toxicity to the patient. Typically, the pharmaceutical compositions of the present invention may include a GnRH receptor antagonist in an amount from 0.1 mg to 500 mg per day dosage depending upon the route of administration, and more typically from 5 mg to 250 mg per day. Appropriate concentrations and dosages can be readily determined by one skilled in the art.

Determination of a therapeutically effective amount or a prophylactically effective amount of the compounds of the invention can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific GnRH mediated disorder involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other coadministered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a GnRH receptor antagonist, diluents, dispersing and surface active agents, binders, and lubricants. One skilled in this art may further formulate the GnRH receptor antagonist in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating sex-hormone-related conditions as discussed above. Such methods include administering of a compound of the present invention to a warm-blooded animal in an amount sufficient to treat the condition. In this context, “treat” includes prophylactic administration. Such methods include systemic administration of a GnRH receptor antagonist of this invention, preferably in the form of a pharmaceutical composition as discussed above. As used herein, systemic administration includes oral and parenteral methods of administration. For oral administration, suitable pharmaceutical compositions of GnRH receptor antagonists include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parenteral administration, the compounds of the present invention can be prepared in aqueous injection solutions which may contain, in addition to the GnRH receptor antagonist, buffers, antioxidants, bacteriostats, and other additives commonly employed in such solutions.

### MODE(S) FOR CARRYING OUT THE INVENTION

The following examples are provided for purposes of illustration, not limitation. In summary, the GnRH receptor antagonists of this invention may be assayed by the general methods disclosed above, while the following examples disclose the synthesis of representative compounds of this invention.

### EXPERIMENTAL DETAILS AND GENERAL PROCESSES

The following table lists the abbreviations used in this paragraph and in the examples section as far as they are not explained within the text body.

| Abbreviation | Meaning |
|---|---|
| Ac | acetyl |
| aq. | aqueous |
| br. s. | broad singlet |
| d | doublet |
| dd | doublet of doublets |
| dt | doublet of triplets |
| DCM | dichloromethane |
| DIPEA | N,N-diisopropylethylamine |
| DMF | N,N-dimethylformamide |
| DMSO | dimethyl sulfoxide |
| eq. | equivalent(s) |
| ESI | electrospray ionization |
| EtOAc | ethyl acetate |
| GP | general procedure |
| HATU | O-(7-azabenzotriazol-1-yl)-1,1,3,3- |
| | tetramethyluronium hexafluorophosphate |
| HOAt | 1-hydroxy-7-azabenzotriazole |
| HPLC | high performance liquid chromatography |
| LCMS | liquid chromatography mass spectrometry |
| LDA | lithium diisopropylamide |
| m | multiplet |
| mc | centred multiplet |
| mCPBA | meta-chloroperoxybenzoic acid |
| MS | mass spectrometry |
| NMR | nuclear magnetic resonance spectroscopy: |
| | chemical shifts (δ) are given in ppm |
| q | quartet |
| Rₜ | retention time |
| r.t. or rt or room temp. | room temperature |
| s | singlet |
| sat. | saturated |
| t | triplet |
| TEA | triethylamine |
| TLC | thin layer chromatography |
| TFA | trifluoroacetic acid |
| THF | tetrahydrofuran |
| UPLC | ultra performance liquid chromatography |
| UPLC-MS | ultra performance liquid chromatography- |
| | mass spectrometry |

NMR peak forms are stated as they appear in the spectra, possible higher order effects have not been considered. Chemical shifts are given in ppm; all spectra were calibrated to solvent residual peak. Integrals are given in integers.

Ultra performance liquid chromatography/liquid chromatography mass spectrometry—methods:

The terms “UPLC-MS (ESI+)” or “UPLC-MS (ESI−)” refer to the following conditions: Instrument: Waters Acquity UPLC-MS SQD 3001; column: Acquity UPLC BEH C18 1.7 50×2.1 mm; eluent A: water+0.1% vol. formic acid (99%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD; or

Instrument: Waters Acquity UPLC-MS SQD 3001; column: Acquity UPLC BEH C18 1.7 50×2.1 mm; eluent A: water+0.05% vol. formic acid (98%), eluent B: acetonitrile+0.05% vol. formic acid (98%); gradient: 0-1.6 min 1-99% B, 1.6-2.0 min 99% B; flow 0.8 mL/min; temperature: 60° C.; injection: 2 μl; DAD scan: 210-400 nm; ELSD; or

Instrument: Waters Acquity UPLC-MS SQD 3001; column: Acquity UPLC BEH C18 1.7 50×2.1 mm; Eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

The separation of enantiomers was performed by preparative chiral HPLC. In the description of the individual examples is referred to the applied HPLC procedure from the following list:

Method A: Dionex: Pump P 580, Gilson: Liquid Handler 215, Knauer: UV-Detector K-2501; Temperature: rt. Columns, solvent system, flow, injection parameters and detection system are specified at the respective example.

Method B: Sepiatec: Prep SFC100; Pressure outlet: 150 bar. Columns, solvent system, flow, temperature, injection parameters and detection system are specified at the respective example.

Method C: Agilent: Prep 1200, 2×Prep Pump G1361A, DLA G2258A, MWD G1365D, Prep FC G1364B; Temperature: rt. Columns, solvent system, flow, injection parameters and detection system are specified at the respective example.

Analytical characterization of enantiomers was performed by analytical chiral HPLC. In the description of the individual examples is referred to the applied HPLC procedure from the following list:

Method D: Waters: Alliance 2695, DAD 996, ESA: Corona; Flow: 1.0 mL/min; Temperature: 25° C.; Injection: 5.0 μL, 1.0 mg/mL ethanol/methanol (1:1). Columns, solvent system and detection system are specified at the respective example.

Method E: Agilent: 1260 AS, MWD, Aurora SFC-Module; Flow: 4.0 mL/min; Pressure outlet: 100 bar; Temperature: 37.5° C.; Injection: 10.0 μL, 1.0 mg/mL ethanol/methanol (1:1). Columns, solvent system and detection system are specified at the respective example.

Method F: Agilent: 1260 AS, MWD, Aurora SFC-Module; Column: Chiralpak ID 5 μm 100×4.6 mm; Solvent: CO₂/2-propanol 65/35; Flow: 4.0 mL/min; Pressure outlet: 150 bar; Temperature: 40° C.; Injection: 10.0 μL, 1.0 mg/mL ethanol/methanol (1:1); Detection: DAD 254 nm.

Chemical names were generated according to the IUPAC rules [ACD/Name Batch ver. 12.00] or using AutoNom2000 as implemented in MDL ISIS Draw [MDL Information Systems Inc. (Elsevier MDL)]. In some cases generally accepted names of commercially available reagents were used in place of IUPAC names or AutoNom2000 generated names. Stereodescriptors are used according to Chemical Abstracts.

Reactions employing microwave irradiation may be run with a Biotage Initiator microwave oven optionally equipped with a robotic unit. The reported reaction times employing microwave heating are intended to be understood as fixed reaction times after reaching the indicated reaction temperature.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the persion skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

The following schemes and general procedures illustrate general synthetic routes to the compounds of general formula (I) of the invention and are not intended to be limiting. It is obvious to the person skilled in the art that the order of transformations as exemplified in Schemes 1 to 4 can be modified in various ways. The order of transformations exemplified in Schemes 1 to 4 is therefore not intended to be limiting. In addition, interconversion of substituents, for example of residues R¹, R² and R³ can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts, Protective Groups in Organic Synthesis, 3rd edition, Wiley 1999).

Compounds of general formula (I) corresponding to formula 6 may be synthesized according to the procedures depicted in Scheme 1 from suitably functionalized carboxylic acids of formula 8 by reaction with appropriate amines of general formula 9:

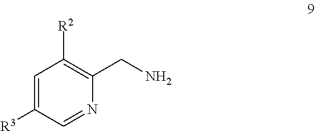

For amide formation, however, all processes that are known from peptide chemistry to the person skilled in the art may be applied. The acids of general formula 8 can be reacted with an appropriate amine of formula 9, for example in the particular form of a hydrochloride salt, in aprotic polar solvents, such as for example DMF, acetonitrile or N-methylpyrrolid-2-one via an activated acid derivative, which is obtainable for example with hydroxybenzotriazole and a carbodiimide such as for example diisopropylcarbodiimide, or else with preformed reagents, such as for example O-(7-azabenzotriazol-1-yl)-1,1,3,3-tetramethyluronium hexafluoro-phosphate (see for example Chem. Comm. 1994, 201-203), or else with activating agents such as dicyclohexylcarbodiimide/N,N-dimethylaminopyridine or N-ethyl-N′,N′-dimethylaminopropylcarbodiimide/N,N-dimethylaminopyridine. The addition of a suitable base such as for example N-methylmorpholine, TEA or DIPEA may be necessary. In certain cases, the activated acid derivative might be isolated prior to reaction with the appropriate amine. Amide formation may also be accomplished via the acid halide (which can be formed from a carboxylic acid by reaction with e.g. oxalyl chloride, thionyl chloride or sulfuryl chloride), mixed acid anhydride (which can be formed from a carboxylic acid by reaction with e.g. isobutylchloroformate), imidazolide (which can be formed from a carboxylic acid by reaction with e.g. carbonyldiimidazole) or azide (which can be formed from a carboxylic acid by reaction with e.g. diphenylphosphorylazide).

Carboxylic acids of general formula 8 in turn may be obtained from carboxylic esters of formula 7 by saponification with inorganic bases such as lithium hydroxide, potassium hydroxide or sodium hydroxide in a suitable solvent such as methanol, THF, water or mixtures thereof at temperatures between 0° C. and the boiling point of the solvent(mixture), typically at room temperature. Alternatively, carboxylic acids of general formula 8 may be directly formed from aryl bromides of general formula 5 under palladium catalyzed carbonylation conditions. Thus, bromides of formula 5 may be reacted in a suitable solvent such as for example dimethyl sulfoxide in the presence of a carbon monoxide source such as for example molybdenum hexacarbonyl or under a carbon monoxide atmosphere at pressures between 1 and 20 bar and in the presence of a palladium catalyst system such as for example palladium(II) acetate/1,1′-bis(diphenylphosphino)ferrocene and a base such as potassium acetate at temperatures between room temperature and the boiling point of the solvent, preferably at 100° C.

Carboxylic esters of general formula 7 may be synthesized from aryl bromides of formula 5 by reaction with an appropriate alcohol under palladium catalyzed carbonylation conditions. Bromides of formula 5 might be reacted in a polar aprotic solvent such as for example dimethylsulfoxide with an appropriate alcohol such as methanol in the presence of a carbon monoxide source such as for example molybdenum hexacarbonyl or under a carbon monoxide atmosphere at pressures between 1 and 20 bar and in the presence of a suitable palladium catalyst such as bis(triphenylphosphine) palladium(II) dichloride and a base such as for example triethylamine at temperatures between room temperature and the boiling point of the solvent, preferably at 100° C.

Alternatively, amides of general formula 6 may be directly synthesized from aryl bromides of formula 5 by reaction with appropriate amines of general formula 9 under palladium catalyzed carbonylation conditions. For this carbonylation all processes that are known to the person skilled in the art may be applied. Bromides of formula 5 can be reacted in a polar aprotic solvent such as for example dioxane with an appropriate amine 9, for example in the particular form of a hydrochloride salt, in the presence of a carbon monoxide source such as for example molybdenum hexacarbonyl or under a carbon monoxide atmosphere at pressures between 1 and 20 bar and in the presence of a palladium catalyst such as for example palladium(II) acetate and a base such as sodium carbonate at temperatures between room temperature and the boiling point of the solvent, preferably at 110° C. It might be necessary to add a ligand such as tri-tert-butylphosphonium tetrafluoroborate to the mixture.

Aryl bromides of general formula 5 in turn may be formed from indolines of general formula 4 by reaction with electrophiles Y of general formula:

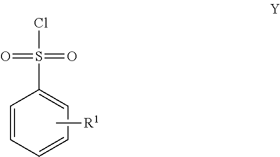

in an organic solvent such as dichloromethane, 1,2-dichloroethane or acetonitrile in the presence of a tertiary amine base such as triethylamine or DIPEA and optionally in the presence of 4-dimethylaminopyridine at temperatures between room temperature and the boiling point of the solvent, typically at 80° C. Alternatively, indolines of general formula 4 may be reacted with electrophiles Y without additional solvent in the presence of a tertiary base such as triethylamine or pyridine at room temperature to give aryl bromides of general formula 5. In the above procedures, the shown electrophiles Y are either commercially available, known compounds or may be formed from known compounds by known methods by a person skilled in the art.

Indolines of general formula 4 may be synthesized from suitably functionalized indolenines of general formulae 3a or 3b by either reduction (3a to 4) or addition of a nucleophile (3b to 4). For reduction, the indolenines 3a may be reacted in a suitable organic solvent such as for example methanol in the presence of a reducing agent such as for example sodium borohydride, sodium (triacetoxy)borohydride or sodium cyanoborohydride at temperatures between 0° C. and the boiling point of the solvent, typically at room temperature. In case of a nucleophilic addition, the indolenines 3b may be reacted in a suitable organic solvent such as for example THF with a nucleophile of formula Z:

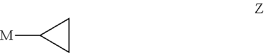

where M is a metal as for example preferably lithium or magnesium, and most preferably in the form of a Grignard reagent, like a MgBr derivative, at temperatures between 0° C. and the boiling point of the solvent, typically at room temperature (see WO06/090261, pp. 67-68 for a similar procedure). It might be necessary to add a Lewis acid such as boron trifluoride diethyl etherate to the mixture.

Alternatively, 3b may be reacted in a suitable organic solvent such as for example toluene with a nucleophile Z being a Grignard reagent, particularly a MgBr derivative, in the presence of copper(I) chloride at temperatures between room temperature and the boiling point of the solvent, typically at 120° C. to give indolines of general formula 4 (see J. Chem. Soc. Perkin Trans. 1, 1988, 3243-3247).

Indolenines of general formulae 3a or 3b may be obtained from suitably functionalized carbonyl compounds of general formulae 2a or 2b and a phenylhydrazine of formula 1 by condensation to give a hydrazone intermediate and a subsequent cyclization reaction (Fischer indole synthesis) in an organic solvent such as for example chloroform or acetic acid and in the presence of a suitable acid such as for example trifluoroacetic acid or hydrochloric acid at temperatures between 0° C. and the boiling point of the solvent (see for example Liu et al., Tetrahedron 2010, 66, 3, 573-577 or WO10/151737, p. 224 for similar procedures). In the above procedures, carbonyl compounds of general formulae 2a or 2b and phenyl-hydrazines of general formula 1 are either commercially available, known compounds or may be formed from known compounds by known methods by a person skilled in the art.

The obtained indolines of general formulae 4, 5, 6, 7 and 8 are chiral and may be separated into their diastereomers and/or enantiomers by e.g. chiral HPLC or crystallization.

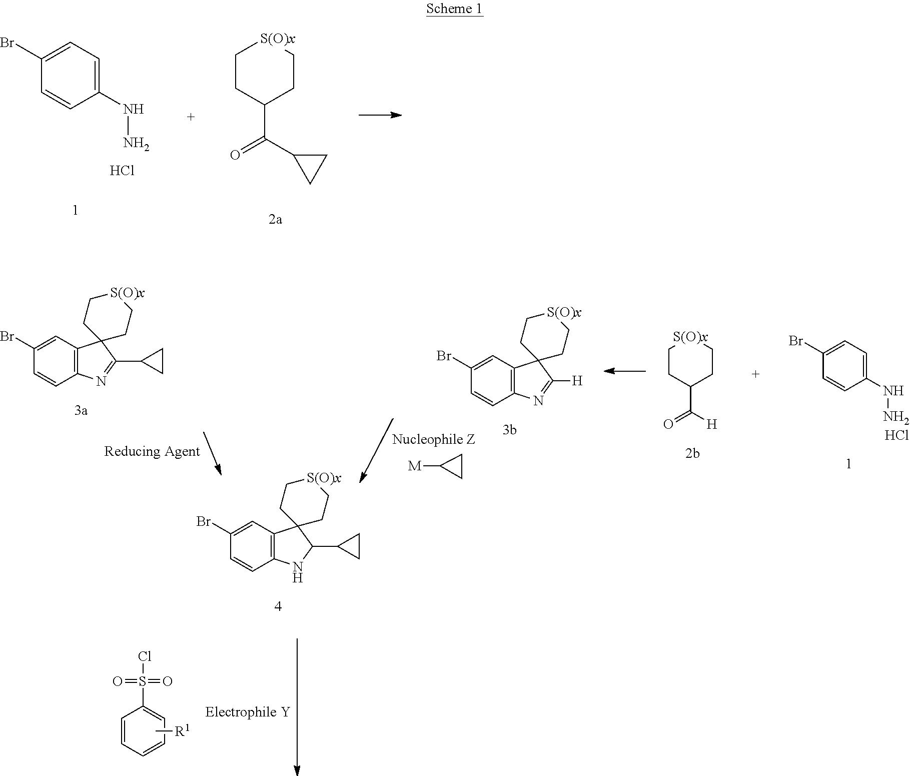

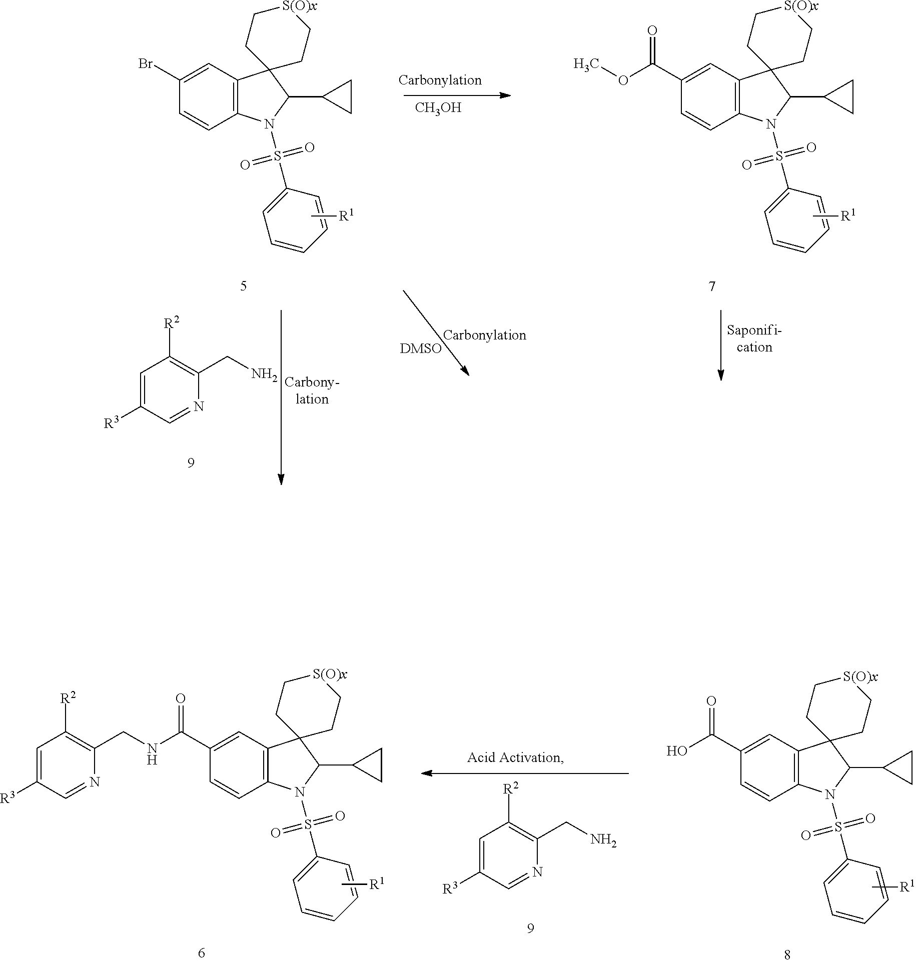

Scheme 1 General procedures for the preparation of compounds of general formula (I) corresponding to formula 6; R¹, R² and R³ are as defined in the description and claims of this invention.

Instead of using carbonyl compounds of general formula 2b in the indolenine synthesis (see Scheme 1) enol ethers of general formula 10 can be applied in certain cases to obtain indolenines of general formula 3b as depicted in Scheme 2. The reaction conditions are comparable to those described in Scheme 1 for the syntheses of 3b from 1 and 2b. Enol ethers of formula 10 are either commercially available, known compounds or may be formed from known compounds by known methods by a person skilled in the art.

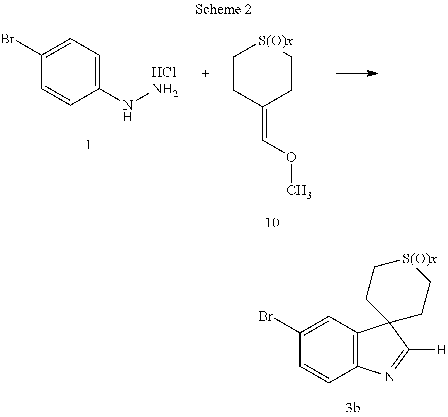

Scheme 2 General procedure for the preparation of compounds of general formula 3b.

In case of spirotetrahydrothiopyranes the sulfur atom might be oxidized as depicted in Scheme 3. Sulfones of general formula 13 may be obtained from suitably functionalized spirotetrahydrothiopyranes of general formula 11 by twofold oxidation applying peroxides. Thus, spirotetrahydrothiopyranes of formula 11 may be reacted in organic solvents such as for example dichloromethane or acetonitril with peroxides such as for example 3-chloroperoxybenzoic acid or urea hydrogen peroxide in the presence of trifluoroacetic anhydride at temperatures between 0° C. and the boiling point of the solvent, preferably at room temperature. Alternatively, sulfones of formula 13 may be synthesized from sulfoxides of general formula 12 under similar reaction conditions as described for the syntheses of 13 from 11.

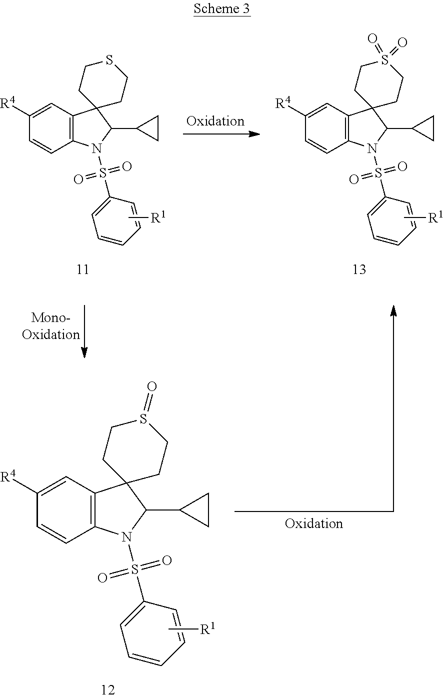

Scheme 3 General procedures for the preparation of compounds of general formula 12 and 13; R¹ is as defined in the description and claims of this invention. The procedures are favorable for the synthesis of compounds wherein R⁴ is hydrogen, halogen, C(O)OH, C(O)O—C₁-C₄-alkyl or

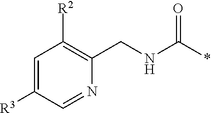

wherein * indicates the point of attachment of said group and R² and R³ are as defined in the description and claims of this invention.

Sulfoxides of general formula 12 may be obtained from spirotetrahydrothiopyranes of general formula 11 by mono-oxidation in an organic solvent such as for example acetonitrile with periodic acid and a catalytic amount of iron(III) chloride at temperatures between 0° C. and the boiling point of the solvent, preferably at room temperature.

Compounds of general formula (Ia) corresponding to formula 20 may be synthesized according to the procedures shown in Scheme 4. The compounds of formulae 20, 21 and 22 can be obtained in an analogous way as described for the compounds of formulae 6, 7 and 8 in Scheme 1.

Sulfones of general formula 19 may be synthesized from compounds of general formula 18 by oxidation with peroxides. The procedures are analogous to those described for the syntheses of 13 from 11 in Scheme 3.

Sulfonamides of general formula 18 may be obtained from suitably functionalized indolines of general formula 17 by reaction with electrophiles Y as described for the syntheses of 5 from 4 in Scheme 1.

Indolines of general formula 17 may be synthesized from suitably functionalized indolenines of general formula 16 by reaction in a suitable organic solvent such as for example THF with a nucleophile Z as defined above and being preferably a Grignard reagent, particularly a MgBr derivative, in the presence of a Lewis acid such as boron trifluoride diethyl etherate at temperatures between 0° C. and the boiling point of the solvent, typically at room temperature. Alternatively, 16 may be reacted in a suitable organic solvent such as for example toluene with a nucleophile Z being a Grignard reagent, particularly a MgBr derivative, in the presence of copper(I) chloride at temperatures between room temperature and the boiling point of the solvent, typically at 120° C. (see J. Chem. Soc. Perkin Trans. 1, 1988, 3243-3247). Indolenines of general formula 16 may be obtained from suitably functionalized carbonyl compounds of general formula 14 and a phenylhydrazine of formula 1 by condensation in an analogous way as described for the syntheses of 3b from 1 and 2b in Scheme 1. Alternatively, indolenines of general formula 16 may be synthesized from suitably functionalized enol ethers of general formula 15 and a phenylhydrazine of formula 1 as described in Scheme 2.

It is obvious to the person skilled in the art that the oxidations as exemplified in Scheme 3 and Scheme 4 can be done at different stages of the syntheses to obtain compounds of the present invention.

The obtained indolines of general formulae 17, 18, 19, 20, 21 and 22 are chiral and may be separated into their enantiomers by e.g. chiral HPLC or crystallization.

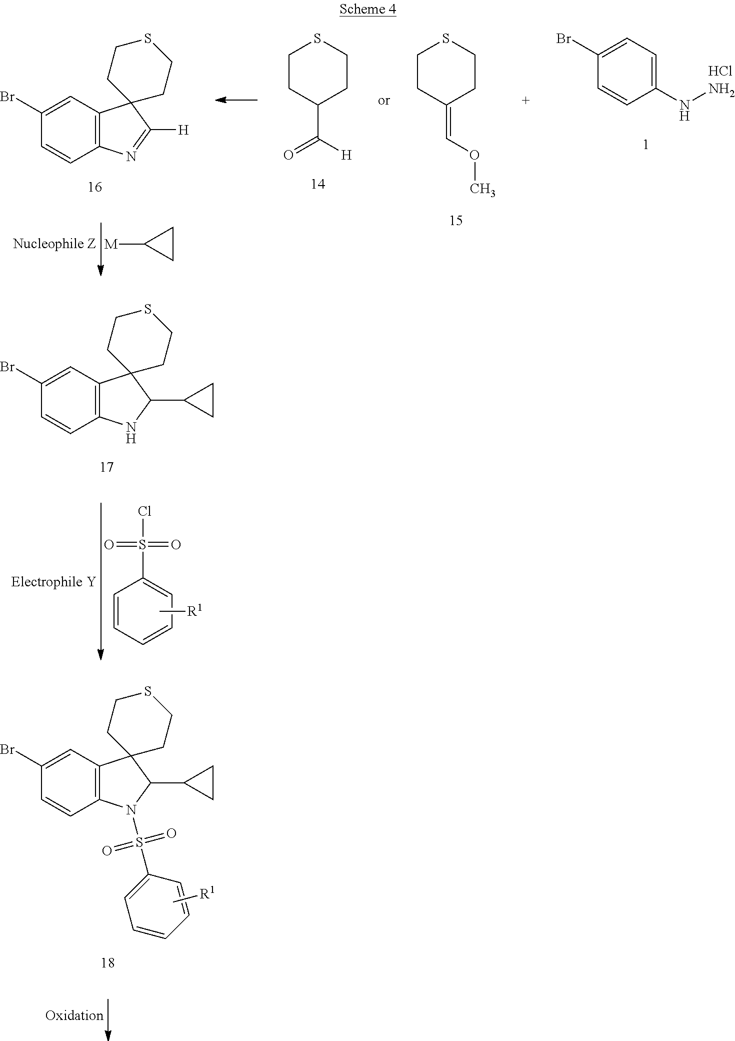

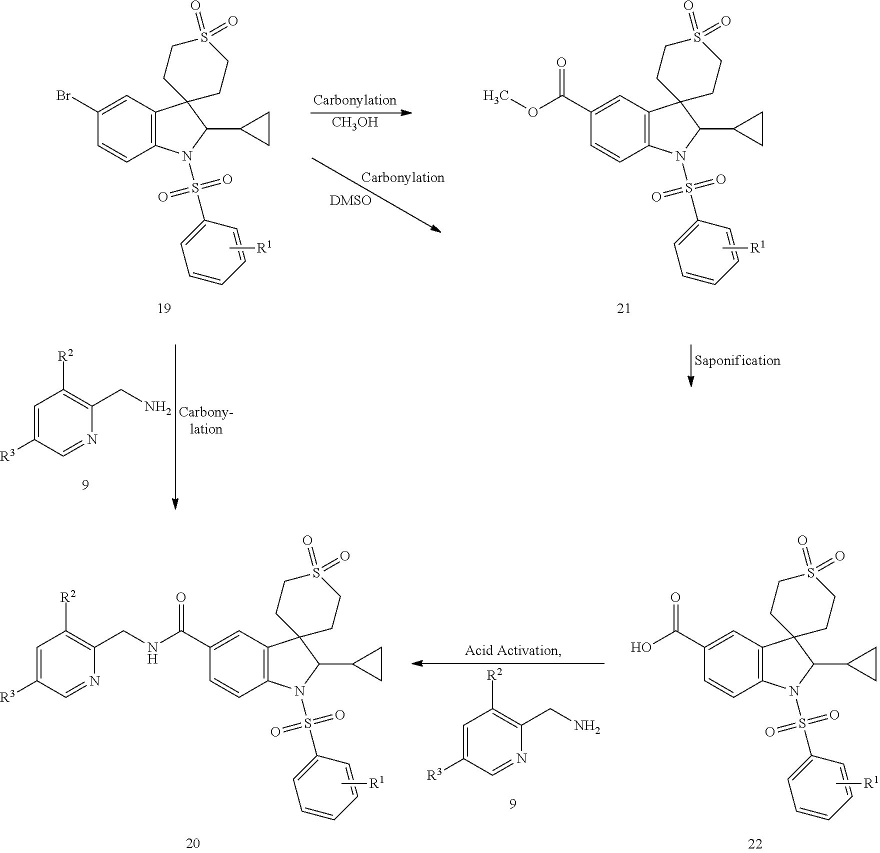

Scheme 4 General procedures for the preparation of compounds of general formula 20; R¹, R², and R³ are as defined in the description and claims of this invention. The procedures are favorable for the synthesis of compounds of general formula (Ia) corresponding to formula 20.

### General Procedures

In the subsequent paragraphs detailed general procedures for the synthesis of key intermediates and compounds of the present invention are described.

### General Procedure 1 (GP 1): Indolenine Formation (3a and 3b, Schemes 1 and 2)

### Method 1 (GP 1.1): Similar to Liu et al., Tetrahedron 2010, 66, 3, 573-577 or WO10/151737, p. 224.

To a stirred solution of 1 eq. of hydrazine 1 and 1 eq. of carbonyl compound 2a or 2b or enol ether 10 in chloroform at 0° C., 3.3 eq. of trifluoroacetic acid are added dropwise. The reaction mixture is heated to 50° C. until TLC and/or LCMS indicate complete consumption of the starting material (18 h) and then cooled to room temperature. An aqueous solution of ammonia (25%) is carefully added to reach a pH of ˜8. The mixture is poured into water and extracted with dichloromethane. The combined organic layers are washed with water, dried with sodium sulfate and the solvents removed in vacuo. The crude product is taken to the next step without further purification.

Method 2 (GP 1.2): Indolenine Formation in Acetic Acid/Aq. Hydrochloric Acid

To a stirred solution of 1 eq. of hydrazine 1 in acetic acid (2 mL/mmol) 1 eq. of concentrated hydrochloric acid (aq.) is added at rt. After 5 minutes of stirring, 1 eq. of carbonyl compound 2a or 2b or enol ether 10 is added at rt, the reaction mixture heated to 100° C. until TLC and/or LCMS indicate (nearly) complete consumption of the starting material (4-24 h) and then cooled to room temperature. An aqueous solution of ammonia (25%) is carefully added to reach a pH of ˜8. The mixture is poured into water and extracted with dichloromethane. The combined organic layers are washed with water, dried with sodium sulfate and the solvents removed in vacuo. The crude product is taken to the next step without further purification.

General Procedure 2 (GP 2): Reduction of Indolenine (3a→4, Scheme 1)

To a stirred solution of indolenine 3a in methanol, 4 eq. of sodium borohydride are carefully added at rt. The reaction is stirred at rt until TLC and/or LCMS indicate complete consumption of the starting material (1 h) and then concentrated in vacuo. The residue is taken up with water, acidified with aq. hydrochloric acid (1 M) to a pH of ˜5 and extracted with ethyl acetate. The combined organic layers are washed with brine, dried with sodium sulfate and the solvents removed in vacuo. The crude product is purified by flash chromatography or preparative HPLC.

General Procedure 3 (GP 3): Grignard Reaction (Nucleophile Addition, 3b→4, Scheme 1) Similar to WO06/090261, Pp. 67-68.

To a stirred solution of indolenine 3b in THF, 1 eq. of boron trifluoride diethylether complex is added dropwise at 0° C. After 5 min of stirring, 3 eq. of the corresponding Grignard reagent (commercial solution in THF or prepared from the respective alkyl bromide according to standard procedures) are added dropwise, keeping the temperature of the mixture at 5-10° C. The mixture is allowed to warm to room temperature and stirred until TLC and/or LCMS indicate complete consumption of the starting material (3 h). Then sat. aqueous ammonium chloride solution is added and the mixture partitioned between ethyl acetate and water. The aqueous phase is extracted with ethyl acetate, the combined organic phases are washed with brine, dried with sodium sulfate, concentrated and purified via flash chromatography (SiO₂-hexane/ethyl acetate).

### General Procedure 4 (GP 4): Sulfonamide Formation (4→5, Scheme 1)

### Method 1 (GP 4.1): Sulfonamide Formation in 1,2-Dichloroethane

To a solution of indoline 4 in 1,2-dichloroethane 2 eq. of sulfonyl chloride and 5 eq. of triethylamine are added at rt and the mixture is stirred at 80° C. for 18-24 h. If needed, further 2 eq. of sulfonyl chloride and 3 eq of triethylamine may be added and the mixture is stirred for additional 18 h. The reaction mixture is partitioned between water and dichloromethane, extracted with dichloromethane, the combined organic layers are washed with water, dried with sodium sulfate, concentrated and purified via flash chromatography (SiO₂-hexane/ethyl acetate).

### Method 2 (GP 4.2): Sulfonamide Formation in Pyridine

A mixture of indoline 4, 2 eq. of sulfonyl chloride and 6 eq. of pyridine is stirred at rt for 18-24 h. The reaction mixture is partitioned between water and dichloromethane, extracted with dichloromethane, the combined organic layers are washed with water, dried with sodium sulfate, concentrated and purified via flash chromatography (SiO₂-hexane/ethyl acetate).

### General Procedure 5 (GP 5): Oxidation to Sulfone (11→13, Scheme 3)

Method 1 (GP 5.1): Oxidation with mCPBA

To a solution of sulfide 11 in dichloromethane, 3 eq. of 3-chloroperoxybenzoic acid are added at 0° C. The mixture is stirred until TLC and/or LCMS indicate complete consumption of the starting material (4 h) and then partitioned between dichloromethane and sat. aqueous sodium hydrocarbonate solution. The organic layer is washed with sodium hydrocarbonate solution, dried with sodium sulfate and concentrated in vacuo. The crude product is purified via flash chromatography (SiO₂-hexane/ethyl acetate).

Method 2 (GP 5.2): Oxidation with Urea Hydrogen Peroxide

6 Eq. trifluoroacetic anhydride are dissolved in acetonitril (5-6 mL/mmol) at 0° C. and 8 eq. of urea hydrogen peroxide are slowly added. After 20 min stirring at rt, a solution of 1 eq. of sulfide 11 in acetonitrile (3.5 mL/mmol) is added dropwise and the mixture stirred for ca. 2 h at rt. In case of incomplete conversion, further up to 8 eq. of urea hydrogen peroxide and the according amount of trifluoroacetic anhydride may be added. After complete conversion, the mixture is partitioned between water and dichloromethane. The aqueous layer is extracted with dichloromethane, the combined organic layers are washed with water and dried with sodium sulfate. The solvents are removed in vacuo and the crude product is purified by flash chromatography to obtain the desired sulfone.

Method 3 (GP 5.3): Oxidation with Oxone®

To a solution of sulfide 11 in a mixture of tetrahydrofurane and methanol (1:1), a solution of 4 eq of Oxone® in water (0.15-0.35 M) is added at 0° C. The mixture is stirred at 0° C. until TLC and/or LCMS indicate complete consumption of the starting material (2 h) and then partitioned between water and ethyl acetate. The layers are separated, the aqueous layer is extracted with ethyl acetate, the combined organic layers washed with brine, dried with sodium sulfate and the solvents removed in vacuo. The obtained crude product is purified via flash chromatography (SiO₂-hexane/ethyl acetate).

### General Procedure 6 (GP 6): Carbonylation to Yield Methylester (5→7, Scheme 1)

The aryl bromide 5 is placed into a steel autoclave under argon atmosphere and dissolved in a 10:1 mixture of methanol and dimethyl sulfoxide (ca. 30 mL/mmol). 0.2 eq. of trans-bis(triphenylphosphine) palladium(II) dichloride and 2.5 eq. of triethylamine are added and the mixture is purged 3 times with carbon monoxide. The mixture is stirred for 30 min at 20° C. under a carbon monoxide pressure of ca. 9.5 bar. The autoclave is set under vacuum again, then a carbon monoxide pressure of ca. 8.6 bar is applied and the mixture heated to 100° C. until TLC and/or LCMS indicate complete consumption of the starting material (22 h), yielding a maximum pressure of ca. 12.2 bar. The reaction is cooled to rt, the pressure released and the reaction mixture concentrated in vacuo and redissolved in ethyl acetate/water. The layers are separated, the aqueous phase extracted with ethyl acetate, the combined organic layers washed with water and brine, then dried with sodium sulfate and the solvents removed in vacuo. The crude product is purified by flash chromatography (SiO₂-hexane/ethyl acetate).

### General Procedure 7 (GP 7): Saponification of Ester (7→8, Scheme 1)

The methyl ester 7 is dissolved in a 1:1 mixture of THF and a 2M aqueous lithium hydroxide solution (ca. 30 mL/mmol) and stirred at rt until TLC and/or LCMS indicate complete consumption of the starting material (18 h). The mixture is set to pH 4 by addition of 2M aqueous hydrochloric acid and extracted with ethyl acetate. The combined organic layers are washed with brine, dried with sodium sulfate and concentrated in vacuo. The product is used without further purification.

### General Procedure 8 (GP 8): Carbonylation to Yield Carboxylic Acid (5→8, Scheme 1)

The aryl bromide 5 is placed into a steel autoclave under argon atmosphere and dissolved in dimethyl sulfoxide (ca. 25 mL/mmol). 5 mol % of palladium(II) acetate, 0.2 eq. of 1,1′-bis(di-phenylphosphino)ferrocene and 4 eq. of potassium acetate are added and the mixture is purged 3 times with carbon monoxide. The mixture is stirred for 30 min at 20° C. under a carbon monoxide pressure of ca. 10.5 bar. The autoclave is set under vacuum again, then a carbon monoxide pressure of ca. 11 bar is applied and the mixture heated to 100° C. until TLC and/or LCMS indicate complete consumption of the starting material (22 h), yielding a maximum pressure of ca. 13.5 bar. The reaction is cooled to rt, the pressure released and the reaction mixture given to a mixture of 2 M HCl_{aq} in ice-water. After stirring for 20 min, the formed precipitate is filtered off, washed with water and redissolved in dichloromethane. The organic layer is washed with water, dried with magnesium sulfate and the solvent removed in vacuo. The obtained crude product is taken to the next step without further purification.

### General Procedure 9 (GP 9): Amide Formation (8→6, Scheme 1)

### Method 1 (GP 9.1): Amide Formation In Situ

The carboxylic acid 8 is dissolved in DMF and 1.5 eq. of the corresponding amine component, 1.5 eq. of HATU and 3 eq. of triethylamine are added. The reaction mixture is stirred at rt until TLC and/or LCMS indicate complete consumption of the starting material (2-24 h), then water is added. The formed precipitate is filtered off, washed with water and taken up with methylene chloride. The organic phase is washed with water, dried with magnesium sulfate and the solvent removed in vacuo. If appropriate, the product is purified by preparative HPLC or flash chromatography.

Method 2 (GP 9.2): Amide Formation after Isolation of Active Ester (HOAt Ester)

The carboxylic acid 8 is dissolved in DMF, 1.5 eq. of HATU and 1.5 eq. of triethylamine are added. The reaction mixture is stirred at rt until TLC and/or LCMS indicate complete consumption of the starting material (2-3 h), then water is added. The formed precipitate is filtered off, washed with water, dissolved in dichloromethane or ethyl acetate or a mixture thereof, dried and concentrated in vacuo to give the HOAt ester.

The HOAt ester, 2 eq. of the corresponding amine component and—if a hydrochloride is used as amine component—1.5 eq. of triethylamine are stirred in acetonitrile or a mixture of acetonitrile and N-methyl-2-pyrrolidone at 55-80° C. until TLC and/or LCMS indicate complete consumption of the HOAt ester (1-30 h). Then the reaction mixture is partitioned between ethyl acetate and water. The layers are separated, the water phase extracted with ethyl acetate, the combined organic layers washed with water and brine, then dried with sodium sulfate and the solvents removed in vacuo. If appropriate, the product is purified by preparative HPLC or flash chromatography.

### General Procedure 10 (GP 10): Carbonylation to Yield Amides Directly (5→6, Scheme 1)

Method 1 (GP 10.1): Amide Formation with Molybdenum Hexacarbonyl

To a solution of aryl bromide 5 in 1,4-dioxane (containing ca. 1% water) 3 eq. of the corresponding amine, 1 eq. of molybdenum hexacarbonyl, 3 eq. of sodium carbonate, 0.1 eq. of tri-tert-butylphosphonium tetrafluoroborate and 0.1 eq. of palladium(II) acetate are added. The reaction mixture is vigorously stirred at 120-140° C. until TLC and/or LCMS indicate complete consumption of the starting material (18 h). Alternatively, microwave irradiation (200 W, 20 min, 140° C., 1.2 bar) can be applied. The mixture is cooled to rt, solids are filtered off and rinsed with ethyl acetate. The filtrate is washed with water and brine, dried with sodium sulfate and concentrated in vacuo. The crude product is purified by flash chromatography (SiO₂-hexane/ethyl acetate) and if appropriate additionally by preparative HPLC.

Method 2 (GP 10.2): Amide Formation with Gaseous Carbon Monoxide

The aryl bromide 5 is placed into a steel autoclave under argon atmosphere and dissolved in THF (ca. 30 mL/mmol). 3 eq. of the corresponding amine, 0.2 eq. of trans-bis(triphenylphosphine) palladium(II) dichloride dichloromethane complex and 2.35 eq. of triethylamine are added and the mixture is purged 3 times with carbon monoxide. The mixture is stirred for 30 min at 20° C. under a carbon monoxide pressure of ca 13 bar. The autoclave is set under vacuum again, then a carbon monoxide pressure of ca 13 bar is applied and the mixture heated to 100-120° C. until TLC and/or LCMS indicate complete consumption of the starting material (22 h), yielding a maximum pressure of ca 18 bar. It might be necessary to repeat the heating under CO pressure after adding additional palladium catalyst to drive the reaction to completion. The reaction is cooled to rt, the pressure released and the reaction mixture filtrated. The residue is washed with THF and the combined filtrates concentrated in vacuo. The crude product is purified by flash chromatography (SiO₂-hexane/ethyl acetate) and if appropriate additionally by preparative HPLC.

### General Procedure 11 (GP 11): Oxidation Sulfide→Sulfoxide (11→12, Scheme 3)

To a solution of sulfide 11 in acetonitrile 0.13 eq. of iron(III) chloride are added at rt. After 15 min stirring, 1.1 eq. of periodic acid is added and the mixture stirred for further 45 min. The mixture is partitioned between water and ethyl acetate. The pH is adjusted to ˜pH 10 by the addition of aqueous sat. sodium hydrocarbonate solution. The layers are separated, the aqueous phase extracted with ethyl acetate, the combined organic layers are washed with brine, dried with sodium sulfate and the solvents evaporated. The crude product is purified by flash chromatography or preparative HPLC.

### SYNTHESIS OF KEY INTERMEDIATES

### Intermediate A.1

### Preparation of 5-Bromo-2′,3′,5′,6′-Tetrahydrospiro[Indole-3,4′-Thiopyran]

### Access Via Carbonyl Compound: Step 1a Swern Oxidation

### Preparation of 3,4,5,6-tetrahydro-2H-thiopyran-4-carbaldehyde

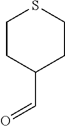

1.4 eq. oxalyl chloride (6.72 g, 52.9 mmol) were dissolved in 200 mL methylene chloride and the solution cooled to −65° C. 2 eq. dimethyl sulfoxide (5.91 g, 75.6 mmol), dissolved in 30 mL methylene chloride were added dropwise within 10 min, so that the temperature didn't exceed −50° C. After 15 min, 1 eq. tetrahydrothiopyran-4-methanol (5.00 g, 37.8 mmol), dissolved in 30 mL methylene chloride, were added dropwise within 5 min at max. −45° C. The mixture was stirred for 1 h, warming to −30° C. 3 eq. triethylamine (11.5 g, 113 mmol) were added dropwise and the mixture was allowed to warm up to room temperature. After stirring 1 h, the mixture was poured into water and extracted with methylene chloride. The combined organic layers were washed with water, dried with sodium sulfate, the solvents removed in vacuo and the crude product (5.70 g, 98%) was directly put forward to the next step.

### Access Via Enol Ether: Step 1b Wittig Reaction (WO09/007747, Pp. 60-61)

### Preparation of 4-(methoxymethylene)-3,4,5,6-tetrahydro-2H-thiopyran

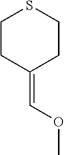

A mixture of (methoxymethyl)triphenylphosphonium chloride (885 g, 2.58 mol, 1.50 eq.) in THF (1300 mL) was cooled to −50° C. and LDA (1.29 L of a 2 M solution in THF/Heptane/Ethylbenzene, 2.58 mol, 1.50 eq.) was added dropwise keeping the temperature below −20° C. After 15 min at −20° C. the deep red reaction mixture was cooled to −40° C. and a solution of tetrahydrothiopyran-4-one (200 g, 1.72 mol, 1.00 eq) in THF (1000 mL) was added dropwise. After 15 min at −40° C. the mixture was allowed to reach rt and was stirred overnight. The reaction mixture was filtered, concentrated in vacuo and filtered again. The obtained filtrate was purified by distillation (B.p. 60° C., 0.02 mbar) to give the title compound (125 g, 50%). ¹H-NMR (300 MHz, CDCl₃): Shift [ppm]=2.27-2.30 (m, 2H), 2.52-2.55 (m, 2H), 2.59-2.62 (m, 4H), 3.55 (s, 3H), 5.82 (s, 1H). UPLC-MS (ESI+): [M+H]⁺=145.

### Step 2 Fischer indole synthesis

### Preparation of 5-bromo-2′,3′,5′,6′-tetrahydrospiro[indole-3,4′-thiopyran]

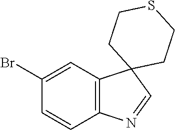 [SMILES: Brc1ccc2c(c1)C1(C=N2)CCSCC1]

According to GP 1.1 1 eq. of 4-bromo-phenylhydrazine hydrochloride (8.96 g, 40.1 mmol) and 1 eq. 3,4,5,6-tetrahydro-2H-thiopyran-4-carbaldehyde (5.80 g, 40 mmol) or, alternatively, 1 eq. of 4-(methoxymethylene)-3,4,5,6-tetrahydro-2H-thiopyran were dissolved in 250 mL chloroform. The solution was cooled to 0° C. and 3.3 eq. trifluoroacetic acid (15.8 g) were added dropwise. The reaction was heated to 50° C. for 18 h, then cooled to room temperature. An aqueous solution of ammonia (25%) was carefully added to reach a pH of about 8. The mixture was poured into water and extracted with methylene chloride. The combined organic layers were washed with water, dried with sodium sulfate and the solvents removed. The product was take to the next step without further purification. UPLC-MS (ESI+): [M+H]⁺=282/284 (Br isotope pattern).

### Intermediate B.1

### Preparation of 5-bromo-2-cyclopropyl-1,2,2′,3′,5′,6′-hexahydrospiro[indole-3,4′-thiopyran]

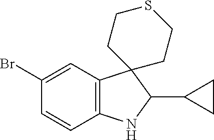 [SMILES: Brc1ccc2c(c1)C1(CCSCC1)C(C1CC1)N2]

According to GP 3 intermediate A.1 (8.82 g, 27.2 mmol), 81.6 mmol cyclopropylmagnesium bromide (0.5 M in THF) and 1 eq (3.86 g) borontrifluoride etherate were reacted in 100 mL THF to yield 3.50 g (32%) of intermediate B.1. ¹H-NMR (300 MHz, DMSO-d6): Shift [ppm]=0.08-0.19 (m, 1H), 0.32-0.42 (m, 2H), 0.43-0.54 (m, 1H), 0.77-0.88 (m, 1H), 1.58-1.66 (m, 1H), 1.81-1.88 (m, 1H), 1.93-2.00 (m, 1H), 2.12-2.20 (m, 1H), 2.57-2.76 (m, 4H), 2.80 (d, 1H), 5.77 (s, br, 1H), 6.40 (d, 1H), 7.02 (dd, 1H), 7.15 (d, 1H). UPLC-MS (ESI+): [M+H]⁺=324/326 (Br isotope pattern).

The racemic material of intermediate B.1 was analytically characterized by HPLC (method D with Column: Chiralpak IA 3 μm 100×4.6 mm; Solvent: ethanol/methanol 50:50 (v/v) or hexane/ethanol 70:30 (v/v); Detection: DAD 254 nm):

Intermediate B.1.1: Rₜ=2.83 min (ethanol/methanol 50:50) or 2.60 min (hexane/ethanol 70.30); enantiomer 1

Intermediate B.1.2: Rₜ=3.68 min (ethanol/methanol 50:50) or 3.52 min (hexane/ethanol 70.30); enantiomer 2

### Intermediate C.1

### Preparation of 5-bromo-2-cyclopropyl-1-[(4-fluorophenyl)sulfonyl]-1,2,2′,3′,5′,6′-hexahydrospiro[indole-3,4′-thiopyran]

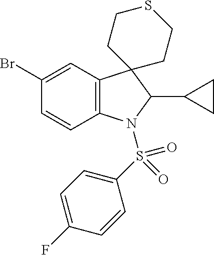 [SMILES: O=S(=O)(c1ccc(F)cc1)N1c2ccc(Br)cc2C2(CCSCC2)C1C1CC1]

According to GP 4.1 indoline B.1 (8.88 mmol) was reacted with 5 eq. triethylamine and 3 eq. 4-fluorobenzenesulfonyl chloride (CAS No. [349-88-2]) in 180 mL 1,2-dichloroethane at 80° C. for 18 h, leading to 80% conversion (by LCMS). Further 3 eq. triethylamine and 2 eq. 4-fluorobenzenesulfonyl chloride were added and stirred for further 24 h at 80° C. to drive the reaction to completion. Isolated yield: 52%. ¹H-NMR (300 MHz, DMSO-d6): Shift [ppm]=0.19 (d, 1H), 0.30-0.45 (m, 2H), 0.51-0.61 (m, 1H), 0.66-0.75 (m, 1H), 0.88-1.02 (m, 2H), 1.94 (d, 1H), 2.03-2.13 (m, 1H), 2.23-2.31 (m, 1H), 2.56 (d, 1H), 2.69-2.86 (m, 2H), 3.98 (d, 1H), 7.33-7.42 (m, 5H), 7.80-7.84 (m, 2H). UPLC-MS (ESI+): [M+H]⁺=482/484 (Br isotope pattern).

The enantiomers of the racemic material of intermediate C.1 were separated by chiral preparative HPLC (method A with Column: Chiralpak IA 5 μm 250×30 mm; Solvent: hexane/2-propanol 70:30 (v/v); Flow: 50 mL/min; Injection: 0.8 mL/run, 64 mg/mL CH₂C₁₂; Detection: UV 254 nm) and analytically characterized by HPLC (method D with Column: Chiralpak IA 3 μm 100×4.6 mm; Solvent: hexane/2-propanol 70:30 (v/v); Detection: DAD 254 nm):

Intermediate C.1.1; (2S)-5-bromo-2-cyclopropyl-1-[(4-fluorophenyl)sulfonyl]-1,2,2′,3′,5′,6′-hexahydrospiro[indole-3,4′-thiopyran]: Rₜ=2.29 min (enantiomer 1)

Intermediate C.1.2; (2R)-5-bromo-2-cyclopropyl-1-[(4-fluorophenyl)sulfonyl]-1,2,2′,3′,5′,6′-hexahydrospiro[indole-3,4′-thiopyran]: Rₜ=3.23 min (enantiomer 2)

### Intermediate C.2

### Preparation of 5-bromo-2-cyclopropyl-1-[(3-methoxyphenyl)sulfonyl]-1,2,2′,3′,5′,6′-hexahydrospiro[indole-3,4′-thiopyran]

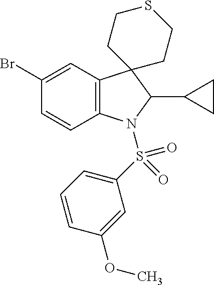 [SMILES: COc1cccc(S(=O)(=O)N2c3ccc(Br)cc3C3(CCSCC3)C2C2CC2)c1]

C.2 was prepared in analogy to intermediate C.1 according to GP 4.1 starting from B.1 and 3-methoxybenzenesulfonyl chloride (CAS No. [10130-74-2]). UPLC-MS (ESI+): [M+H]⁺=494/496 (Br isotope pattern).

### Intermediate C.3

### Preparation of 4-[(5-bromo-2-cyclopropyl-2′,3′,5′,6′-tetrahydrospiro[indole-3,4′-thiopyran]-1(2H)-yl)sulfonyl]benzonitrile

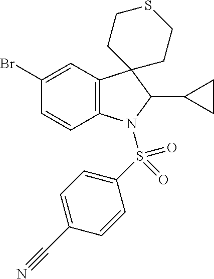 [SMILES: N#Cc1ccc(S(=O)(=O)N2c3ccc(Br)cc3C3(CCSCC3)C2C2CC2)cc1]

C.3 was prepared according to GP 4.2 starting from B.1 and 4-cyanobenzenesulfonyl chloride (CAS No. [60958-06-7]). UPLC-MS (ESI+): [M+H]⁺=489/491 (Br isotope pattern).

### Intermediate C.4

### Preparation of 3-[(5-bromo-2-cyclopropyl-2′,3′,5′,6′-tetrahydrospiro[indole-3,4′-thiopyran]-1(2H)-yl)sulfonyl]benzonitrile

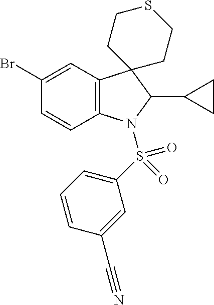 [SMILES: N#Cc1cccc(S(=O)(=O)N2c3ccc(Br)cc3C3(CCSCC3)C2C2CC2)c1]

C.4 was prepared according to GP 4.2 starting from B.1 and 3-cyanobenzenesulfonyl chloride (CAS No. [56542-67-7]). UPLC-MS (ESI+): [M+H]⁺=489/491 (Br isotope pattern).

### Intermediate C.5

### Preparation of 5-bromo-2-cyclopropyl-1-{[3-(trifluoromethoxy)phenyl]sulfonyl}-1,2,2′,3′,5′,6′-hexahydrospiro[indole-3,4′-thiopyran]

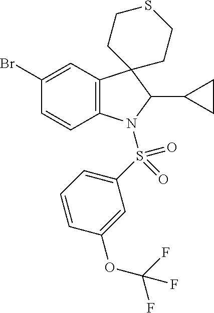 [SMILES: O=S(=O)(c1cccc(OC(F)(F)F)c1)N1c2ccc(Br)cc2C2(CCSCC2)C1C1CC1]

C.5 was prepared according to GP 4.2 starting from B.1 and 3-trifluoromethoxybenzene-sulfonyl chloride (CAS No. [220227-84-9]). UPLC-MS (ESI+): [M+H]⁺=548/550 (Br isotope pattern).

### Intermediate C.6

### Preparation of 5-bromo-2-cyclopropyl-1-{[3-(difluoromethoxy)phenyl]sulfonyl}-1,2,2′,3′,5′,6′-hexahydrospiro[indole-3,4′-thiopyran]

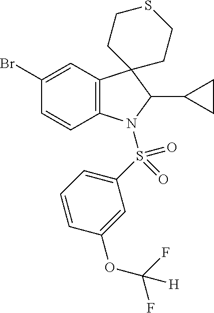 [SMILES: O=S(=O)(c1cccc(OC(F)F)c1)N1c2ccc(Br)cc2C2(CCSCC2)C1C1CC1]

C.6 was prepared according to GP 4.2 starting from B.1 and 3-difluoromethoxybenzene-sulfonyl chloride (CAS No. [351003-38-8]). UPLC-MS (ESI+): [M+H]⁺=530/532.

### Intermediate C.7

### Preparation of 5-bromo-2-cyclopropyl-1-{[4-(difluoromethoxy)phenyl]sulfonyl}-1,2,2′,3′,5′,6′-hexahydrospiro[indole-3,4′-thiopyran]

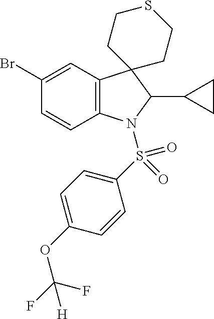 [SMILES: O=S(=O)(c1ccc(OC(F)F)cc1)N1c2ccc(Br)cc2C2(CCSCC2)C1C1CC1]

C.7 was prepared according to GP 4.2 starting from B.1 and 4-difluoromethoxybenzene-sulfonyl chloride (CAS No. [351003-34-4]). UPLC-MS (ESI+): [M+H]⁺=530/532 (Br isotope pattern).

### Intermediate C.8

### Preparation of 4-[(5-bromo-2-cyclopropyl-2′,3′,5′,6′-tetrahydrospiro[indole-3,4′-thiopyran]-1(2H)-yl)sulfonyl]benzamide

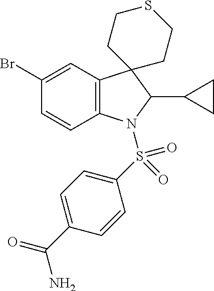 [SMILES: NC(=O)c1ccc(S(=O)(=O)N2c3ccc(Br)cc3C3(CCSCC3)C2C2CC2)cc1]

C.8 was prepared according to GP 4.2 starting from B.1 and 4-carbamoylbenzenesulfonyl chloride (CAS No. [885526-86-3]). ¹H-NMR (300 MHz, DMSO-d6): Shift [ppm]=0.09-0.13 (m, 1H), 0.34-0.51 (m, 2H), 0.56-0.65 (m, 1H), 0.72-0.81 (m, 1H), 0.90-1.00 (m, 2H), 1.87-1.92 (m, 1H), 2.06-2.16 (m, 1H), 2.29-2.34 (m, 1H), 2.56-2.61 (m, 1H), 2.76-2.89 (m, 2H), 3.99-4.06 (m, 1H), 7.39-7.48 (m, 3H), 7.62 (br. s., 1H), 7.84-7.87 (m, 2H), 7.94-7.97 (m, 2H), 8.14 (br. s., 1H). UPLC-MS (ESI+): [M+H]⁺=507/509 (Br isotope pattern).

### Intermediate C.9

### Preparation of 5-bromo-1-[(4-chlorophenyl)sulfonyl]-2-cyclopropyl-1,2,2′,3′,5′,6′-hexahydrospiro[indole-3,4′-thiopyran]

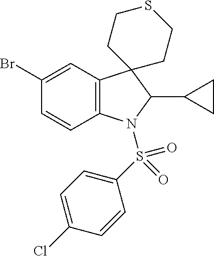 [SMILES: O=S(=O)(c1ccc(Cl)cc1)N1c2ccc(Br)cc2C2(CCSCC2)C1C1CC1]

C.9 was prepared in a slight modification to GP 4.2 starting from B.1 (3.0 g) with 1.5 eq. of 4-chlorobenzenesulfonyl chloride (CAS No. [98-60-2]) and 9 eq. of pyridine. After stirring for 20 h at rt the reaction mixture was poured on a water-ice mixture (350 mL), stirred for 20 min, the precipitate filtered off and washed with water (50 mL). The obtained solid was taken up with methylene chloride, dried with magnesium sulfate and the solvent removed in vacuo to give the desired sulfonamide. UPLC-MS (ESI+): [M+H]⁺=498/500 (Br/CI isotope pattern).

### Intermediate D.1

### Preparation of 5-bromo-2-cyclopropyl-1-[(4-fluorophenyl)sulfonyl]-1,2,2′,3′,5′,6′-hexahydrospiro[indole-3,4′-thiopyran] 1′,1′-dioxide

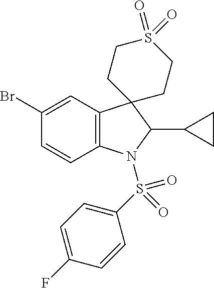 [SMILES: O=S1(=O)CCC2(CC1)c1cc(Br)ccc1N(S(=O)(=O)c1ccc(F)cc1)C2C1CC1]

According to GP 5.2 8.84 g (18.3 mmol) of intermediate C.1 were oxidized with 13.8 g (8 eq.) urea hydrogen peroxide/23 g (6 eq.) trifluoroacetic anhydride to yield 9.25 g (98%) of the desired sulfone. ¹H-NMR (300 MHz, DMSO-d6): Shift [ppm]=0.13-0.22 (m, 1H), 0.32-0.48 (m, 2H), 0.50-0.60 (m, 1H), 0.74-0.83 (m, 1H), 0.89-1.01 (m, 1H), 1.41 (dt, 1H), 2.34-2.58 (m, 3H), 3.09-3.17 (m, 2H), 3.56 (dt, 1H), 4.26 (d, 1H), 7.34-7.47 (m, 5H), 7.80-7.88 (m, 2H). UPLC-MS (ESI+): [M+H]⁺=514/516 (Br isotope pattern).

Alternatively, 8 mmol of intermediate C.1 (3.86 g) were oxidized according to GP 5.1 with 3 eq (4.19 g) of 3-chloroperoxybenzoic acid for 4 h at 0° C. to yield 2.3 g (56%) of the desired sulfone (identical by Rₜ on UPLC-MS).

The enantiomers of the racemic material of intermediate D.1 were separated by chiral preparative HPLC (method A with Column: Chiralpak IA 5 μm 250×30 mm; Solvent: hexane/2-propanol 80:20 (v/v); Flow: 50 mL/min; Injection: 0.4 mL/run, 83 mg/mL CH₂Cl₂/THF; Detection: UV 254 nm) and analytically characterized by HPLC (method D with Column: Chiralpak IA 3 μm 100×4.6 mm; Solvent: hexane/2-propanol 70:30 (v/v); Detection: DAD 254 nm):

Intermediate D.1.1; (2S)-5-bromo-2-cyclopropyl-1-[(4-fluorophenyl)sulfonyl]-1,2,2′,3′,5′,6′-hexahydrospiro[indole-3,4′-thiopyran] 1′,1′-dioxide: Rₜ=3.50 min (enantiomer 1)

Intermediate D.1.2; (2R)-5-bromo-2-cyclopropyl-1-[(4-fluorophenyl)sulfonyl]-1,2,2′,3′,5′,6′-hexahydrospiro[indole-3,4′-thiopyran] 1′,1′-dioxide: Rₜ=4.40 min (enantiomer 2)

Alternatively, intermediate D.1.1 was obtained from intermediate C.1.1 according to GP 5.2 by oxidation with 8 eq. trifluoroacetic anhydride and 10 eq. urea hydrogen peroxide for 30 min at rt. The obtained product was identical to the above obtained enantiomer 1 by analytical chiral HPLC (method D as above):

Intermediate D.1.1: Rₜ=3.57 min (enantiomer 1)

Alternatively, intermediate D.1.2 was obtained from intermediate C.1.2 according to GP 5.2 by oxidation with 8 eq. trifluoroacetic anhydride and 10 eq. urea hydrogen peroxide for 30 min at rt. The obtained product was identical to the above obtained enantiomer 2 by analytical chiral HPLC (method D as above):

Intermediate D.1.2: Rₜ=4.42 min (enantiomer 2)

### Intermediate D.2

### Preparation of 5-bromo-2-cyclopropyl-1-[(3-methoxyphenyl)sulfonyl]-1,2,2′,3′,5′,6′-hexahydrospiro[indole-3,4′-thiopyran] 1′,1′-dioxide

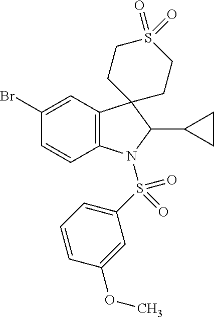 [SMILES: COc1cccc(S(=O)(=O)N2c3ccc(Br)cc3C3(CCS(=O)(=O)CC3)C2C2CC2)c1]

D.2 was prepared in analogy to intermediate D.1 according to GP 5.2 starting from C.2. UPLC-MS (ESI+): [M+H]⁺=526/528 (Br isotope pattern).

### Intermediate D.3

### Preparation of 4-[(5-bromo-2-cyclopropyl-1′,1′-dioxido-2′,3′,5′,6′-tetrahydrospiro[indole-3,4′-thiopyran]-1(2H)-yl)sulfonyl]benzonitrile

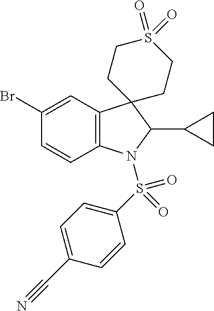 [SMILES: N#Cc1ccc(S(=O)(=O)N2c3ccc(Br)cc3C3(CCS(=O)(=O)CC3)C2C2CC2)cc1]

D.3 was prepared in analogy to intermediate D.1 according to GP 5.2 starting from C.3. ¹H-NMR (300 MHz, DMSO-d6): Shift [ppm]=0.08 (d, 1H), 0.33-0.43 (m, 1H), 0.44-0.61 (m, 2H), 0.75-0.84 (m, 1H), 0.90-1.01 (m, 1H), 1.33-1.44 (m, 1H), 2.33-2.43 (m, 1H), 2.51-2.67 (m, 2H), 3.04-3.19 (m, 2H), 3.48-3.60 (m, 1H), 4.27 (d, 1H), 7.39-7.49 (m, 3H), 7.93 (d, 2H), 8.01 (d, 2H). UPLC-MS (ESI+): [M+H]⁺=522/524 (Br isotope pattern).

### Intermediate D.4

### Preparation of 3-[(5-bromo-2-cyclopropyl-1′,1′-dioxido-2′,3′,5′,6′-tetrahydrospiro[indole-3,4′-thiopyran]-1(2H)-yl)sulfonyl]benzonitrile

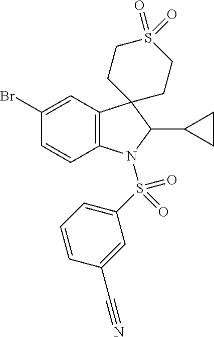 [SMILES: N#Cc1cccc(S(=O)(=O)N2c3ccc(Br)cc3C3(CCS(=O)(=O)CC3)C2C2CC2)c1]

D.4 was prepared in analogy to intermediate D.1 according to GP 5.2 starting from C.4. UPLC-MS (ESI+): [M+H]⁺=522/524 (Br isotope pattern).

### Intermediate D.5

### Preparation of 5-bromo-2-cyclopropyl-1-{[3-(trifluoromethoxy)phenyl]sulfonyl}-1,2,2′,3′,5′,6′-hexahydrospiro[indole-3,4′-thiopyran] 1′,1′-dioxide

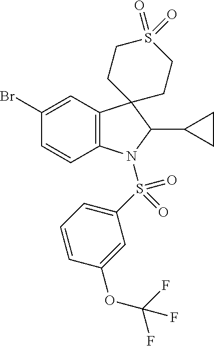 [SMILES: O=S1(=O)CCC2(CC1)c1cc(Br)ccc1N(S(=O)(=O)c1cccc(OC(F)(F)F)c1)C2C1CC1]

D.5 was prepared in analogy to intermediate D.1 according to GP 5.2 starting from C.5. UPLC-MS (ESI+): [M+H]⁺=580/582 (Br isotope pattern).

### Intermediate D.6

### Preparation of 5-bromo-2-cyclopropyl-1-{[3-(difluoromethoxy)phenyl]sulfonyl}-1,2,2′,3′,5′,6′-hexahydrospiro[indole-3,4′-thiopyran] 1′,1′-dioxide

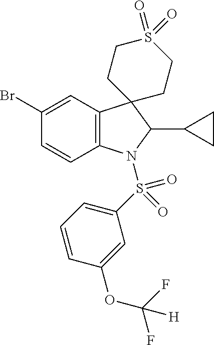 [SMILES: O=S1(=O)CCC2(CC1)c1cc(Br)ccc1N(S(=O)(=O)c1cccc(OC(F)F)c1)C2C1CC1]

D.6 was prepared in analogy to intermediate D.1 according to GP 5.2 starting from C.6. UPLC-MS (ESI+): [M+H]⁺=562/564 (Br isotope pattern).

### Intermediate D.7

### Preparation of 5-bromo-2-cyclopropyl-1-{[4-(difluoromethoxy)phenyl]sulfonyl}-1,2,2′,3′,5′,6′-hexahydrospiro[indole-3,4′-thiopyran] 1′,1′-dioxide

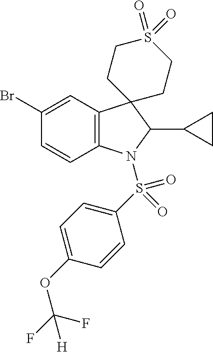 [SMILES: O=S1(=O)CCC2(CC1)c1cc(Br)ccc1N(S(=O)(=O)c1ccc(OC(F)F)cc1)C2C1CC1]

D.7 was prepared in analogy to intermediate D.1 according to GP 5.2 starting from C.7. UPLC-MS (ESI+): [M+H]⁺=562/564 (Br isotope pattern).

### Intermediate D.8

### Preparation of 4-[(5-bromo-2-cyclopropyl-1′,1′-dioxido-2′,3′,5′,6′-tetrahydrospiro[indole-3,4′-thiopyran]-1(2H)-yl)sulfonyl]benzamide

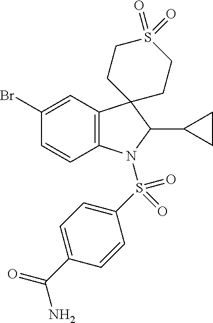 [SMILES: NC(=O)c1ccc(S(=O)(=O)N2c3ccc(Br)cc3C3(CCS(=O)(=O)CC3)C2C2CC2)cc1]

D.8 was prepared in a modification to GP 5.2 starting from C.8. Deviating from GP 5.2 the reaction mixture was filtered upon completion and the obtained residue washed with acetonitrile to get a first crop of product. The filtrate was worked-up as described in GP 5.2 to get a second crop. Both materials were combined and taken to the next step without further purification. ¹H-NMR (400 MHz, DMSO-d6): Shift [ppm]=0.08-0.13 (m, 1H), 0.38-0.45 (m, 1H), 0.48-0.54 (m, 1H), 0.56-0.63 (m, 1H), 0.82-0.88 (m, 1H), 0.95-1.03 (m, 1H), 1.39 (dt, 1H), 2.39-2.56 (m, 3H), 3.16-3.18 (m, 2H), 3.60 (dt, 1H), 4.32 (d, 1H), 7.46-7.48 (m, 3H), 7.60 (br. s., 1H), 7.86-7.88 (m, 2H), 7.93-7.96 (m, 2H), 8.12 (br. s., 1H). UPLC-MS (ESI+): [M+H]⁺=539/541 (Br isotope pattern).

### Intermediate D.9

### Preparation of 5-bromo-1-[(4-chlorophenyl)sulfonyl]-2-cyclopropyl-1,2,2′,3′,5′,6′-hexahydrospiro[indole-3,4′-thiopyran] 1′,1′-dioxide

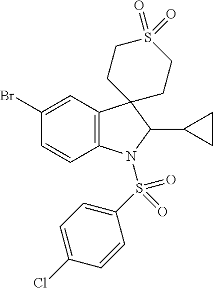 [SMILES: O=S1(=O)CCC2(CC1)c1cc(Br)ccc1N(S(=O)(=O)c1ccc(Cl)cc1)C2C1CC1]

D.9 was prepared in analogy to intermediate D.1 according to GP 5.2 starting from C.9. UPLC-MS (ESI+): [M+H]⁺=530/532 (Br/CI isotope pattern).

### Intermediate E.1

### Preparation of methyl 2-cyclopropyl-1-[(4-fluorophenyl)sulfonyl]-1,2,2′,3′,5′,6′-hexahydrospiro[indole-3,4′-thiopyran]-5-carboxylate 1′,1′-dioxide

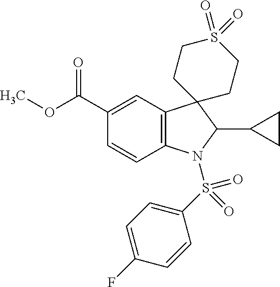 [SMILES: COC(=O)c1ccc2c(c1)C1(CCS(=O)(=O)CC1)C(C1CC1)N2S(=O)(=O)c1ccc(F)cc1]

According to GP 6 4.2 mmol of intermediate D.1 were carbonylated in a mixture of 120 mL methanol, 12 mL DMSO and 1.4 mL triethylamine (10.5 mmol) in the presence of 600 mg trans-bis(triphenylphosphine) palladium(II) dichloride (0.84 mmol). A carbon monoxide pressure of 8.59 bar was applied at 20° C., then the autoclave was heated to 100° C. internal temperature to reach a pressure of 12.2 bar. The reaction was complete after 22 h. Yield: 1.80 g of the desired methyl ester (82%). UPLC-MS (ESI+): [M+H]⁺=494.

### Intermediate E.2

### Preparation of methyl 2-cyclopropyl-1-[(4-fluorophenyl)sulfonyl]-1,2,2′,3′,5′,6′-hexahydrospiro[indole-3,4′-thiopyran]-5-carboxylate

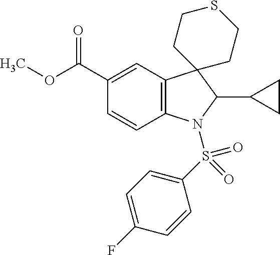 [SMILES: COC(=O)c1ccc2c(c1)C1(CCSCC1)C(C1CC1)N2S(=O)(=O)c1ccc(F)cc1]

According to GP 6 4.2 mmol of intermediate C.1 were carbonylated in a mixture of 95 mL methanol, 9.4 mL DMSO and 1.4 mL triethylamine (10.4 mmol) in the presence of 594 mg trans-bis(triphenylphosphine) palladium(II) dichloride (0.83 mmol). A carbon monoxide pressure of 13.8 bar was applied at 20° C., after evacuation a carbon monoxide pressure of 10 bar was applied and the autoclave was heated to 100° C. internal temperature to reach a pressure of 13.8 bar. The reaction was stopped after 23 h. The product obtained by flash chromatography (SiO₂-hexane/ethyl acetate; 888 mg) was recrystallized from EtOAc to give 526 mg of the desired methyl ester (26%). ¹H-NMR (400 MHz, DMSO-d6): Shift [ppm]=0.30-0.33 (m, 1H), 0.36-0.42 (m, 1H), 0.44-0.50 (m, 1H), 0.58-0.65 (m, 1H), 0.73-0.79 (m, 1H), 0.91-1.05 (m, 2H), 1.99-2.02 (m, 1H), 2.10-2.18 (m, 1H), 2.36-2.40 (m, 1H), 2.62-2.67 (m, 1H), 2.79-2.91 (m, 2H), 3.82 (s, 3H), 4.08 (d, 1H), 7.37-7.42 (m, 2H), 7.61 (d, 1H), 7.69 (d, 1H), 7.88-7.92 (m, 3H). UPLC-MS (ESI+): [M+H]⁺=462.

### Intermediate E.3

### Preparation of methyl 2-cyclopropyl-1-[(4-fluorophenyl)sulfonyl]-1,2,2′,3′,5′,6′-hexahydrospiro[indole-3,4′-thiopyran]-5-carboxylate 1′-oxide

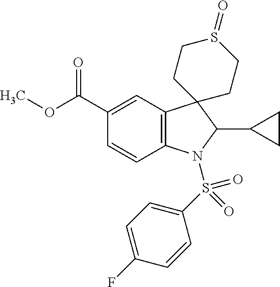 [SMILES: COC(=O)c1ccc2c(c1)C1(CCS(=O)CC1)C(C1CC1)N2S(=O)(=O)c1ccc(F)cc1]

In analogy to GP 11 a solution of 1.04 mmol of intermediate E.2 in 25 mL acetonitrile (prepared by short sonication with ultrasound) was oxidized with 22 mg iron(III) chloride (0.13 eq.) and 261 mg periodic acid (1.1 eq.) to yield 610 mg of the desired product as a 2:1 mixture of sulfoxide diastereomers. The crude product was taken to the next step without further purification. ¹H-NMR (400 MHz, DMSO-d6, major isomer:): Shift [ppm]=−0.12 (d, 1H), 0.27-0.47 (m, 2H), 0.49-0.65 (m, 1H), 0.78-0.86 (m, 1H), 0.93-1.00 (m, 1H), 1.63 (dt, 1H), 2.04-2.08 (m, 1H), 2.31-2.39 (m, 1H), 2.59-2.69 (m, 1H), 2.74-2.81 (m, 1H), 2.90-3.04 (m, 2H), 3.81-3.83 (m, 3H), 4.20 (d, 1H), 7.39-7.47 (m, 2H), 7.63 (d, 1H) [minor isomer: 7.64 (d, 1H)], 7.68 (d, 1H) [minor isomer: 7.75 (d, 1H)], 7.83-7.99 (m, 3H).). UPLC-MS (ESI+): [M+H]⁺=478.

### Intermediate E.4

### Preparation of methyl 2-cyclopropyl-1-[(3-methoxyphenyl)sulfonyl]-1,2,2′,3′,5′,6′-hexahydrospiro[indole-3,4′-thiopyran]-5-carboxylate 1′,1′-dioxide

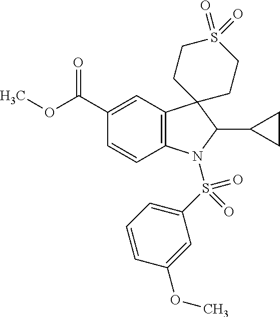 [SMILES: COC(=O)c1ccc2c(c1)C1(CCS(=O)(=O)CC1)C(C1CC1)N2S(=O)(=O)c1cccc(OC)c1]

E.4 was prepared in analogy to intermediate E.1 according to GP 6 starting from D.2. UPLC-MS (ESI+): [M+H]⁺=506.

### Intermediate E.5

### Preparation of methyl 2-cyclopropyl-1-{[3-(trifluoromethoxy)phenyl]sulfonyl}-1,2,2′,3′,5′,6′-hexahydrospiro[indole-3,4′-thiopyran]-5-carboxylate 1′,1′-dioxide

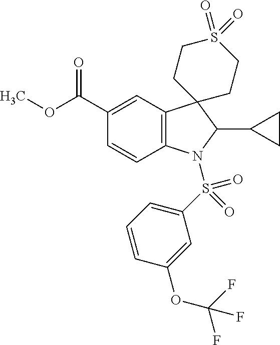 [SMILES: COC(=O)c1ccc2c(c1)C1(CCS(=O)(=O)CC1)C(C1CC1)N2S(=O)(=O)c1cccc(OC(F)(F)F)c1]

E.5 was prepared in analogy to intermediate E.1 according to GP 6 starting from D.5. ¹H-NMR (400 MHz, DMSO-d6): Shift [ppm]=0.19 (d, 1H), 0.32-0.62 (m, 3H), 0.76-0.85 (m, 1H), 0.89-1.02 (m, 1H), 1.40 (dt, 1H), 3.62 (dt, 1H), 3.79 (s, 3H), 4.35 (d, 1H), 7.60-7.96 (m, 7H). UPLC-MS (ESI+): [M+H]⁺=560.

### Intermediate E.6

### Preparation of methyl 2-cyclopropyl-1-{[3-(difluoromethoxy)phenyl]sulfonyl}-1,2,2′,3′,5′,6′-hexahydrospiro[indole-3,4′-thiopyran]-5-carboxylate 1′,1′-dioxide

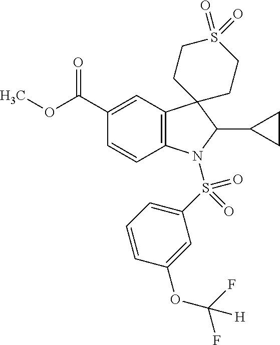 [SMILES: COC(=O)c1ccc2c(c1)C1(CCS(=O)(=O)CC1)C(C1CC1)N2S(=O)(=O)c1cccc(OC(F)F)c1]

E.6 was prepared in analogy to intermediate E.1 according to GP 6 starting from D.6. UPLC-MS (ESI+): [M+H]⁺=542.

### Intermediate E.7

### Preparation of methyl 2-cyclopropyl-1-{[4-(difluoromethoxy)phenyl]sulfonyl}-1,2,2′,3′,5′,6′-hexahydrospiro[indole-3,4′-thiopyran]-5-carboxylate 1′,1′-dioxide

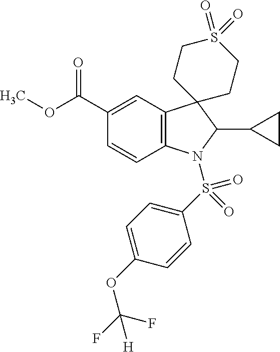 [SMILES: COC(=O)c1ccc2c(c1)C1(CCS(=O)(=O)CC1)C(C1CC1)N2S(=O)(=O)c1ccc(OC(F)F)cc1]

E.7 was prepared in analogy to intermediate E.1 according to GP 6 starting from D.7. UPLC-MS (ESI+): [M+H]⁺=542.

### Intermediate E.8

### Preparation of methyl 1-[(4-chlorophenyl)sulfonyl]-2-cyclopropyl-1,2,2′,3′,5′,6′-hexahydrospiro[indole-3,4′-thiopyran]-5-carboxylate 1′,1′-dioxide

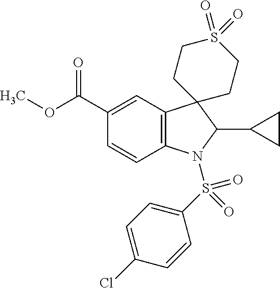 [SMILES: COC(=O)c1ccc2c(c1)C1(CCS(=O)(=O)CC1)C(C1CC1)N2S(=O)(=O)c1ccc(Cl)cc1]

E.8 was prepared in a slight modification to GP 6 starting from D.9 (5.2 g). After concentration of the crude reaction mixture in vacuo water (500 mL) is added, the formed precipitate filtered off and washed with water (80 mL). The obtained solid was taken up with methylene chloride (200 mL), dried with magnesium sulfate and the solvent removed in vacuo. The crude product was recrystallized from EtOAc to give the desired ester (4.2 g). UPLC-MS (ESI+): [M+H]⁺=510/512 (Cl isotope pattern).

### Intermediate F.1

### Preparation of 2-cyclopropyl-1-[(4-fluorophenyl)sulfonyl]-1,2,2′,3′,5′,6′-hexahydrospiro[indole-3,4′-thiopyran]-5-carboxylic acid 1′,1′-dioxide

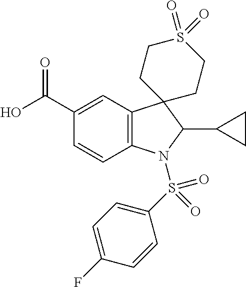 [SMILES: O=C(O)c1ccc2c(c1)C1(CCS(=O)(=O)CC1)C(C1CC1)N2S(=O)(=O)c1ccc(F)cc1]

According to GP 7 1.90 g of intermediate E.1 were hydrolyzed in 130 mL of a 1:1 mixture of THF and 2M aqueous lithium hydroxide solution to yield 1.50 g (77%) of the desired carboxylic acid. UPLC-MS (ESI−): [M−H]⁻=478.

### Intermediate F.2

### Preparation of 2-cyclopropyl-1-[(4-fluorophenyl)sulfonyl]-1,2,2′,3′,5′,6′-hexahydrospiro[indole-3,4′-thiopyran]-5-carboxylic acid 1′-oxide

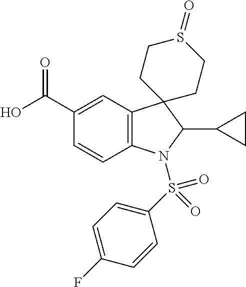 [SMILES: O=C(O)c1ccc2c(c1)C1(CCS(=O)CC1)C(C1CC1)N2S(=O)(=O)c1ccc(F)cc1]

In a slight modification to GP 7 600 mg of intermediate E.3 were hydrolyzed with lithium hydroxide (433 mg, 15 eq.) in 7 mL THF and 3 mL water to yield 530 mg (85%) of the desired carboxylic acid as a 2:1 mixture of sulfoxide diastereomers. The crude product was taken to the next step without further purification. ¹H-NMR (300 MHz, DMSO-d6, major isomer:): Shift [ppm]=−0.14 (d, 1H), 0.30-0.47 (m, 2H), 0.50-0.69 (m, 1H), 0.79-0.87 (m, 1H), 0.92-1.04 (m, 1H), 1.57-1.67 (m, 1H), 2.03-2.08 (m, 1H), 2.33-2.39 (m, 1H), 2.65-2.81 (m, 2H), 2.89-3.05 (m, 2H), 4.19 (d, 1H), 7.39-7.48 (m, 2H), 7.59 (d, 1H) [minor isomer: 7.61 (d, 1H)], 7.66 (d, 1H) [minor isomer: 7.72 (d, 1H)], 7.83-7.97 (m, 3H), 12.88 (br. s., 1H). UPLC-MS (ESI+): [M+H]⁺=464.

### Intermediate F.3

### Preparation of 2-cyclopropyl-1-[(3-methoxyphenyl)sulfonyl]-1,2,2′,3′,5′,6′-hexahydrospiro[indole-3,4′-thiopyran]-5-carboxylic acid 1′,1′-dioxide

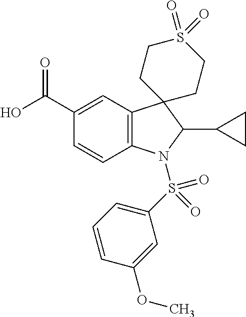 [SMILES: COc1cccc(S(=O)(=O)N2c3ccc(C(=O)O)cc3C3(CCS(=O)(=O)CC3)C2C2CC2)c1]

F.3 was prepared in analogy to intermediate F.1 according to GP 7 starting from E.4. UPLC-MS (ESI−): [M−H]⁻=490.

### Intermediate F.4

### Preparation of 2-cyclopropyl-1-{[3-(trifluoromethoxy)phenyl]sulfonyl}-1,2,2′,3′,5′,6′-hexahydrospiro[indole-3,4′-thiopyran]-5-carboxylic acid 1′,1′-dioxide

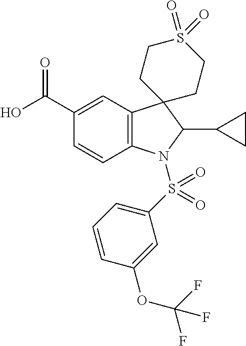 [SMILES: O=C(O)c1ccc2c(c1)C1(CCS(=O)(=O)CC1)C(C1CC1)N2S(=O)(=O)c1cccc(OC(F)(F)F)c1]

F.4 was prepared in analogy to intermediate F.1 according to GP 7 starting from E.5. UPLC-MS (ESI−): [M−H]⁻=544.

### Intermediate F.5

### Preparation of 2-cyclopropyl-1-{[3-(difluoromethoxy)phenyl]sulfonyl}-1,2,2′,3′,5′,6′-hexahydrospiro[indole-3,4′-thiopyran]-5-carboxylic acid 1′,1′-dioxide

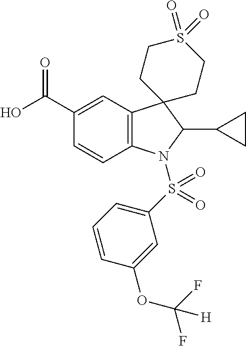 [SMILES: O=C(O)c1ccc2c(c1)C1(CCS(=O)(=O)CC1)C(C1CC1)N2S(=O)(=O)c1cccc(OC(F)F)c1]

F.5 was prepared in analogy to intermediate F.1 according to GP 7 starting from E.6. UPLC-MS (ESI−): [M−H]⁻=526.

### Intermediate F.6

### Preparation of 2-cyclopropyl-1-{[4-(difluoromethoxy)phenyl]sulfonyl}-1,2,2′,3′,5′,6′-hexahydrospiro[indole-3,4′-thiopyran]-5-carboxylic acid 1′,1′-dioxide

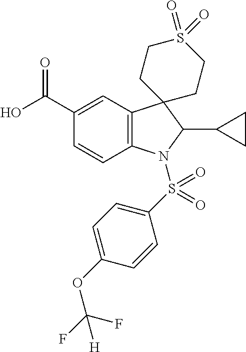 [SMILES: O=C(O)c1ccc2c(c1)C1(CCS(=O)(=O)CC1)C(C1CC1)N2S(=O)(=O)c1ccc(OC(F)F)cc1]

F.6 was prepared in analogy to intermediate F.1 according to GP 7 starting from E.7. UPLC-MS (ESI−): [M−H]⁻=526.

### Intermediate F.7

### Preparation of 1-[(4-cyanophenyl)sulfonyl]-2-cyclopropyl-1,2,2′,3′,5′,6′-hexahydrospiro[indole-3,4′-thiopyran]-5-carboxylic acid 1′,1′-dioxide

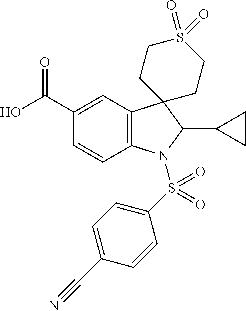 [SMILES: N#Cc1ccc(S(=O)(=O)N2c3ccc(C(=O)O)cc3C3(CCS(=O)(=O)CC3)C2C2CC2)cc1]

F.7 was prepared according to GP 8 starting from D.3. The aryl bromide D.3 (1 g) was placed into a steel autoclave under argon atmosphere and dissolved in dimethyl sulfoxide (30 mL). 25 mg of palladium(II) acetate, 250 mg of 1,1′-bis(diphenylphosphino)ferrocene and 750 mg of potassium acetate were added and the mixture was purged 3 times with carbon monoxide. The mixture was stirred for 30 min at 20° C. under a carbon monoxide pressure of ca. 11.3 bar. The autoclave was set under vacuum again, then a carbon monoxide pressure of ca. 12.69 bar was applied and the mixture heated to 100° C. until TLC and/or LCMS indicate complete consumption of the starting material (24 h), yielding a maximum pressure of ca. 14.9 bar. The reaction was cooled to rt, the pressure released and the reaction mixture given to a mixture of 2 M HCl_{aq} in ice-water. After stirring for 20 min, the formed precipitate was filtered off and washed with water. The obtained crude product was taken to the next step without further purification. UPLC-MS (ESI−): [M−H]⁻=485.

### Intermediate F.8

### Preparation of 1-[(3-cyanophenyl)sulfonyl]-2-cyclopropyl-1,2,2′,3′,5′,6′-hexahydrospiro[indole-3,4′-thiopyran]-5-carboxylic acid 1′,1′-dioxide

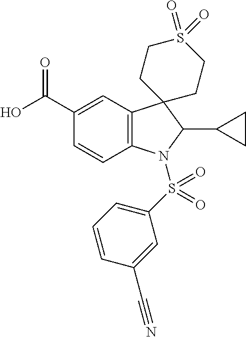 [SMILES: N#Cc1cccc(S(=O)(=O)N2c3ccc(C(=O)O)cc3C3(CCS(=O)(=O)CC3)C2C2CC2)c1]

F.8 was prepared in analogy to intermediate F.7 according to GP 8 starting from D.4. UPLC-MS (ESI−): [M−H]⁻=485.

### Intermediate F.9

### Preparation of 1-[(4-carbamoylphenyl)sulfonyl]-2-cyclopropyl-1,2,2′,3′,5′,6′-hexahydrospiro[indole-3,4′-thiopyran]-5-carboxylic acid 1′,1′-dioxide

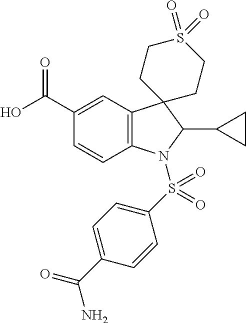 [SMILES: NC(=O)c1ccc(S(=O)(=O)N2c3ccc(C(=O)O)cc3C3(CCS(=O)(=O)CC3)C2C2CC2)cc1]

F.9 was prepared in a modification to GP 8 starting from D.8. Deviating from GP 8 the precipitate obtained upon aqueous work-up was redissolved in ethyl acetate. It was further proceeded as described in GP 8. ¹H-NMR (300 MHz, DMSO-d6): Shift [ppm]=0.16-0.21 (m, 1H), 0.38-0.47 (m, 1H), 0.50-0.65 (m, 2H), 0.83-0.90 (m, 1H), 0.94-1.03 (m, 1H), 1.34-1.44 (m, 1H), 2.50-2.56 (m, 3H), 3.17-3.22 (m, 2H), 3.59-3.69 (m, 1H), 4.38 (d, 1H), 7.60-7.66 (m, 3H), 7.88-7.96 (m, 5H), 8.11 (br. s., 1H), 12.93 (br. s., 1H). UPLC-MS (ESI−): [M−H]⁻=503.

### Intermediate F.10

### Preparation of 1-[(4-chlorophenyl)sulfonyl]-2-cyclopropyl-1,2,2′,3′,5′,6′-hexahydrospiro[indole-3,4′-thiopyran]-5-carboxylic acid 1′,1′-dioxide

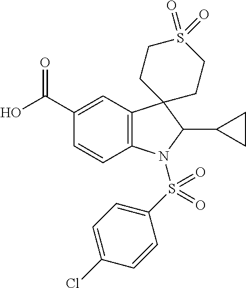 [SMILES: O=C(O)c1ccc2c(c1)C1(CCS(=O)(=O)CC1)C(C1CC1)N2S(=O)(=O)c1ccc(Cl)cc1]

In a slight modification to GP 7 4.2 g of intermediate E.8 were hydrolyzed with lithium hydroxide (2.9 g, 15 eq.) in 47 mL THF and 20 mL water for two days to yield 4.3 g of the desired carboxylic acid. The crude product was taken to the next step without further purification. UPLC-MS (ESI−): [M−H]⁻=494/496 (Cl isotope pattern).

### Intermediate F.11

### Preparation of 2-cyclopropyl-1-[(4-fluorophenyl)sulfonyl]-1,2,2′,3′,5′,6′-hexahydrospiro[indole-3,4′-thiopyran]-5-carboxylic acid

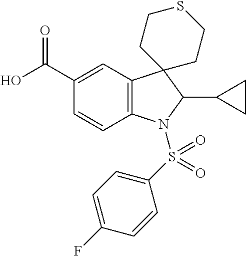 [SMILES: O=C(O)c1ccc2c(c1)C1(CCSCC1)C(C1CC1)N2S(=O)(=O)c1ccc(F)cc1]

F.11 was prepared according to GP 7 starting from ester intermediate E.2 (1.08 g) which was hydrolyzed in a 2M aqueous lithium hydroxide solution (68 mL) for 4 days to yield 1.1 g of the desired carboxylic acid. The crude product was taken to the next step without further purification. ¹H-NMR (300 MHz, DMSO-d6): Shift [ppm]=0.29-0.52 (m, 3H), 0.57-0.66 (m, 1H), 0.72-0.80 (m, 1H), 0.90-1.05 (m, 2H), 1.98-2.03 (m, 1H), 2.08-2.17 (m, 1H), 2.36-2.41 (m, 1H), 2.62-2.66 (m, 1H), 2.78-2.91 (m, 2H), 4.07 (d, 1H), 7.37-7.42 (m, 2H), 7.59 (d, 1H), 7.67 (d, 1H), 7.87-7.92 (m, 3H), 12.86 (br. s., 1H). UPLC-MS (ESI+): [M+H]⁺=448.

Alternatively, intermediate C.1 (4.15 mmol, 2.00 g) was carbonylated according to GP 8 under a carbon monoxide pressure of 16 bar (maximum pressure) overnight at 100° C. to yield 2.6 g (quant.) of the desired carboxylic acid (identical by Rₜ on UPLC-MS) which was not further purified.

Compounds according to the invention:

### Example 1

### 2-cyclopropyl-1-[(4-fluorophenyl)sulfonyl]-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide

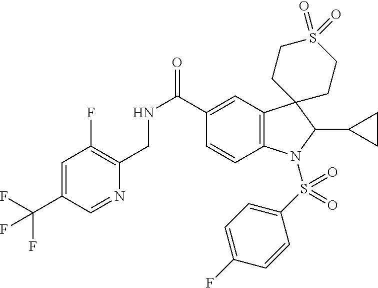 [SMILES: O=C(NCc1ncc(C(F)(F)F)cc1F)c1ccc2c(c1)C1(CCS(=O)(=O)CC1)C(C1CC1)N2S(=O)(=O)c1ccc(F)cc1]

In a slight modification to GP 9.1:100 mg (0.208 mmol) of intermediate F.1 and 83.5 mg (0.313 mmol, 1.5 eq.) 1-[3-fluoro-5-(trifluoromethyl)pyridin-2-yl]methanamine dihydrochloride were reacted with 119 mg (0.313 mmol, 1.5 eq.) HATU in the presence of 131 μL (0.938 mmol, 4.5 eq.) triethylamine in 3.5 mL DMF at rt overnight to yield 122 mg (89%) of the desired amide. The crude product was not further purified. ¹H-NMR (300 MHz, DMSO-d6): Shift [ppm]=0.21-0.33 (m, 1H), 0.34-0.66 (m, 3H), 0.78-0.90 (m, 1H), 0.94-1.08 (m, 1H), 1.46 (dt, 1H), 3.63 (dt, 1H), 4.36 (d, 1H), 4.69 (d, 2H) 7.40 (m, 2H), 7.58 (m, 1H), 7.80-7.96 (m, 4H), 8.28 (m, 1H), 8.79 (s, 1H), 9.14 (t, 1H). UPLC-MS (ESI+): [M+H]⁺=656.

The enantiomers of the racemic material of example 1 were separated by chiral preparative HPLC (method A with Column: Chiralpak IC 5 μm 250×30 mm; Solvent: ethanol/methanol 50:50 (v/v); Flow: 30 mL/min; Injection: 0.4 mL/run, 89 mg/mL CH₂C₁₂; Detection: UV 280 nm) and analytically characterized by HPLC (method D with Column: Chiralpak IC 5 μm 150×4.6 mm; Solvent: ethanol/methanol 50:50 (v/v); Detection: DAD 280 nm):

### Example 1.1

(2S)-2-cyclopropyl-1-[(4-fluorophenyl)sulfonyl]-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide: Rₜ=3.06 min (enantiomer 1)

### Example 1.2

(2R)-2-cyclopropyl-1-[(4-fluorophenyl)sulfonyl]-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide: Rₜ=3.70 min (enantiomer 2)

### Example 2

### N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′-oxide

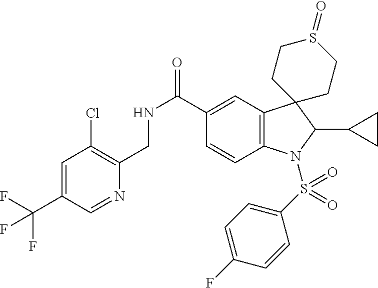 [SMILES: O=C(NCc1ncc(C(F)(F)F)cc1Cl)c1ccc2c(c1)C1(CCS(=O)CC1)C(C1CC1)N2S(=O)(=O)c1ccc(F)cc1]

In a slight modification to GP 9.1: 520 mg (1.12 mmol) of intermediate F.2 (2:1 mixture of sulfoxide diastereomers) and 416 mg (1.68 mmol, 1.5 eq.) 1-[3-chloro-5-(trifluoromethyl)pyridin-2-yl]methanamine hydrochloride (CAS No. [175277-74-4]) were reacted with 640 mg (1.68 mmol, 1.5 eq.) HATU in the presence of 469 μL (3.37 mmol, 3.0 eq.) triethylamine in 6 mL THF at rt overnight. The reaction mixture was taken up with EtOAc and water, the phases separated and the aqueous phase extracted twice with EtOAc. The combined organic layers were dried with sodium sulfate and the solvents removed in vacuo. The crude product was purified by flash chromatography (SiO₂-hexane/ethyl acetate) to yield 512 mg (66%) of the desired amide as a 2:1 mixture of sulfoxide diastereomers. ¹H-NMR (400 MHz, DMSO-d6, major isomer:): Shift [ppm]=−0.14 (d, 1H), 0.38-0.46 (m, 2H), 0.49-0.68 (m, 1H), 0.77-0.87 (m, 1H), 0.96-1.03 (m, 1H), 1.64-1.71 (m, 1H), 2.03-2.08 (m, 1H), 2.35-2.44 (m, 1H), 2.69-3.04 (m, 4H), 4.20 (d, 1H) [minor isomer: 4.14 (d, 1H)], 4.67-4.79 (m, 2H), 7.39-7.44 (m, 2H), 7.57-7.60 (m, 1H) [minor isomer: 7.78 (d, 1H)], 7.84-7.89 (m, 4H) [minor isomer: 7.91-7.95 (m, 2H)], 8.46 (s, 1H), 8.89-8.90 (m, 1H), 9.14 (t, 1H) [minor isomer: 9.00 (t, 1H)]. UPLC-MS (ESI+): [M+H]⁺=656/658 (Cl isotope pattern).

### Example 3

### N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide

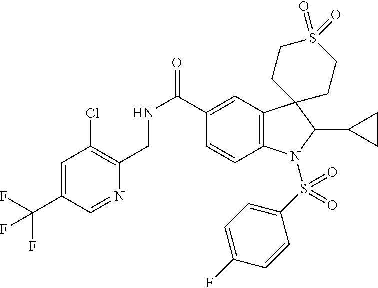 [SMILES: O=C(NCc1ncc(C(F)(F)F)cc1Cl)c1ccc2c(c1)C1(CCS(=O)(=O)CC1)C(C1CC1)N2S(=O)(=O)c1ccc(F)cc1]

According to GP 9.1 100 mg (0.208 mmol) of intermediate F.1 and 77.3 mg (0.313 mmol, 1.5 eq.) 1-[3-chloro-5-(trifluoromethyl)pyridin-2-yl]methanamine hydrochloride (CAS No. [175277-74-4]) were reacted with 119 mg (0.313 mmol, 1.5 eq.) HATU in the presence of 87 μL (0.63 mmol, 3.0 eq.) triethylamine in 2 mL DMF at rt overnight. The crude reaction mixture was directly submitted to prep. HPLC purification to yield 70 mg (50%) of the desired amide. ¹H-NMR (400 MHz, DMSO-d6): Shift [ppm]=0.26-0.30 (m, 1H), 0.38-0.45 (m, 1H), 0.47-0.53 (m, 1H), 0.57-0.64 (m, 1H), 0.82-0.88 (m, 1H), 0.97-1.05 (m, 1H), 1.47 (dt, 1H), 2.46-2.64 (m, 3H), 3.17-3.23 (m, 2H), 3.64 (dt, 1H), 4.37 (d, 1H), 4.68-4.79 (m, 2H), 7.38-7.43 (m, 2H), 7.59 (d, 1H), 7.84 (d, 1H), 7.87-7.92 (m, 3H), 8.46 (d, 1H), 8.90 (d, 1H), 9.11 (t, 1H). UPLC-MS (ESI+): [M+H]⁺=672/674 (Cl isotope pattern).

The enantiomers of the racemic material of example 3 were separated by chiral preparative HPLC (method A with Column: Chiralpak IC 5 μm 250×30 mm; Solvent: ethanol/methanol 50:50 (v/v); Flow: 35 mL/min; Injection: 1.0 mL/run, 39 mg/mL CH₂C₁₂; Detection: UV 280 nm) and analytically characterized by HPLC (method D with Column: Chiralpak IC 3 μm 100×4.6 mm; Solvent: ethanol/methanol 50:50 (v/v); Detection: DAD 280 nm) and specific optical rotation:

### Example 3.1

(2S)—N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide: Rₜ=2.63 min; [α]_{D}²⁰=−101.9°+/−0.13° (C.=10.0 mg/mL, chloroform)

### Example 3.2

(2R)—N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide: Rₜ=3.48 min; [α]_{D}²⁰=+93.0°+/−0.25° (C.=10.0 mg/mL, chloroform)

### Alternative Preparation Via Carbonylation

According to GP 10.2 2.50 g (4.86 mmol) of intermediate D.1 were carbonylated under a carbon monoxide starting pressure of 11.5 bar in the presence of 3.71 g (14.6 mmol, 3.0 eq.) 1-[3-chloro-5-(trifluoromethyl)pyridin-2-yl]methanamine hydrochloride (CAS No. [175277-74-4]), 794 mg (0.97 mmol, 0.20 eq.) trans-bis(triphenylphosphine) palladium(II) dichloride dichloromethane complex and 1.59 mL (11.4 mmol, 2.35 eq.) triethylamine in 126 mL THF at 105° C. for 22 h yielding a maximum pressure of ca 16 bar. The crude product was purified by flash chromatography (SiO₂-hexane/ethyl acetate) to give 2.1 g (63%) of the desired amide which was identical to example 3 by ¹H-NMR and UPLC-MS.

Analogously, according to GP 10.2 280 mg (0.544 mmol) of intermediate D.1.1 were carbonylated under a carbon monoxide starting pressure of 14 bar in the presence of 402 mg (1.63 mmol, 3.0 eq.) 1-[3-chloro-5-(trifluoromethyl)pyridin-2-yl]methanamine hydrochloride (CAS No. [175277-74-4]), 89 mg (0.11 mmol, 0.20 eq.) trans-bis(triphenylphosphine) palladium(II) dichloride dichloromethane complex and 178 μL (1.28 mmol, 2.35 eq.) triethylamine in 15 mL THF at 100° C. for 22 h yielding a maximum pressure of ca 18 bar. The carbonylation procedure was repeated twice for 22 h at 100° C. after adding each time 89 mg (0.11 mmol, 0.20 eq.) trans-bis(triphenylphosphine) palladium(II) dichloride dichloromethane complex to drive the reaction to completion. The crude product was purified by flash chromatography (SiO₂-hexane/ethyl acetate) followed by prep. HPLC to give 25 mg (7%) of the desired amide which was identical to example 3.1 by analytical chiral HPLC (method D as above) and optical rotation.

### Example 3.1

Rₜ=2.56 min; [α]_{D}²⁰=−101.9°+/−0.16° (C.=9.4 mg/mL, chloroform)

Analogously, according to GP 10.2 250 mg (0.486 mmol) of intermediate D.1.2 were carbonylated under a carbon monoxide starting pressure of 13 bar in the presence of 370 mg (1.46 mmol, 3.0 eq.) 1-[3-chloro-5-(trifluoromethyl)pyridin-2-yl]methanamine hydrochloride (CAS No. [175277-74-4]), 79 mg (0.097 mmol, 0.20 eq.) trans-bis(triphenylphosphine) palladium(II) dichloride dichloromethane complex and 159 μL (1.14 mmol, 2.35 eq.) triethylamine in 15 mL THF at 120° C. overnight yielding a maximum pressure of ca 17 bar. The crude product was purified by flash chromatography (SiO₂-hexane/ethyl acetate) followed by prep. HPLC to give 96 mg (27%) of the desired amide which was identical to example 3.2 by analytical chiral HPLC (method D as above) and optical rotation.

### Example 3.2

Rₜ=3.51 min; [α]_{D}²⁰=+90.9°+/−0.31° (C.=10.0 mg/mL, chloroform)

### Example 4

### N-{[5-chloro-3-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide

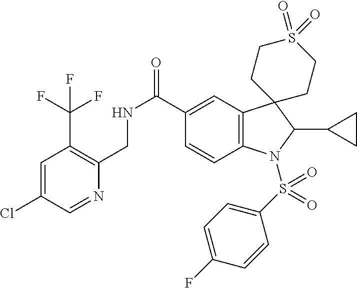 [SMILES: O=C(NCc1ncc(Cl)cc1C(F)(F)F)c1ccc2c(c1)C1(CCS(=O)(=O)CC1)C(C1CC1)N2S(=O)(=O)c1ccc(F)cc1]

According to GP 9.2 530 mg (1.11 mmol) of intermediate F.1 were reacted with 630 mg (1.66 mmol, 1.5 eq.) HATU in the presence of 231 μL (1.66 mmol, 1.5 eq.) triethylamine in 20 mL DMF at rt for 1 h. The obtained HOAt ester (370 mg, 0.551 mmol) was subsequently reacted with 272 mg (1.10 mmol, 2.0 eq.) 1-[5-chloro-3-(trifluoromethyl)pyridin-2-yl]methanamine hydrochloride in the presence of 115 μL (0.826 mmol, 1.5 eq.) triethylamine in 20 mL acetonitrile at 55° C. for 18 h. The obtained crude product was purified by flash chromatography (SiO₂-hexane/ethyl acetate) to give 270 mg (36%) of the desired amide. ¹H-NMR (300 MHz, DMSO-d6): Shift [ppm]=0.24-0.28 (m, 1H), 0.38-0.64 (m, 3H), 0.81-0.89 (m, 1H), 0.95-1.05 (m, 1H), 1.42-1.51 (m, 1H), 2.55-2.64 (m, 3H), 3.20-3.23 (m, 2H), 3.59-3.69 (m, 1H), 4.37 (d, 1H), 4.63-4.76 (m, 2H), 7.38-7.44 (m, 2H), 7.59 (d, 1H), 7.84-7.92 (m, 4H), 8.37 (d, 1H), 8.88 (d, 1H), 9.10 (t, 1H). UPLC-MS (ESI+): [M+H]⁺=672/674 (Cl isotope pattern).

The enantiomers of the racemic material of example 4 were separated by chiral preparative HPLC (method B with Column: Chiralpak ID 5 μm 250×20 mm; Solvent: CO₂/2-propanol 65/35; Flow: 80 mL/min; Temperature: 40° C.; Injection: 0.2 or 1.1 mL/run, 180 mg/mL CH₂Cl₂/CHCl₃2:1; Detection: UV 254 nm) and analytically characterized by HPLC (method F):

### Example 4.1

Rₜ=1.98 min (enantiomer 1)

### Example 4.2

Rₜ=4.34 min (enantiomer 2)

### Example 5

### 2-cyclopropyl-1-[(4-fluorophenyl)sulfonyl]-N-{[5-methyl-3-(trifluoromethyl)pyridin-2-yl]methyl}-1,2,2′,3′,5′,6′-hexahydrospiro[indole-3,4′-thiopyran]-5-carboxamide 1′,1′-dioxide

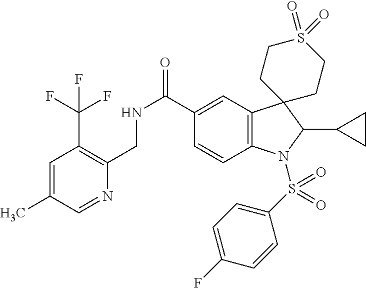 [SMILES: Cc1cnc(CNC(=O)c2ccc3c(c2)C2(CCS(=O)(=O)CC2)C(C2CC2)N3S(=O)(=O)c2ccc(F)cc2)c(C(F)(F)F)c1]

In a slight modification to GP 9.1: 135 mg (0.282 mmol) of intermediate F.1 and 109 mg (0.422 mmol, 1.5 eq.) 1-[5-methyl-3-(trifluoromethyl)pyridin-2-yl]methanamine were reacted with 161 mg (0.422 mmol, 1.5 eq.) HATU in the presence of 59 μL (0.42 mmol, 3.0 eq.) triethylamine in 1.5 mL DMF at rt overnight. The reaction mixture was taken up with EtOAc, the organic phase washed with water, dried with sodium sulfate and the solvents removed in vacuo. The crude product was purified by prep. HPLC purification to yield 61 mg (33%) of the desired amide. ¹H-NMR (300 MHz, DMSO-d6): Shift [ppm]=0.27-0.32 (m, 1H), 0.38-0.45 (m, 1H), 0.47-0.64 (m, 2H), 0.81-0.88 (m, 1H), 0.94-1.03 (m, 1H), 1.42-1.53 (m, 1H), 2.08 (s, 3H), 2.50-2.59 (m, 3H), 3.23 (br. s., 2H), 3.57-3.68 (m, 1H), 4.35 (d, 1H), 4.47-4.61 (m, 2H), 7.37-7.43 (m, 2H), 7.59 (d, 1H), 7.78 (d, 1H), 7.87-7.94 (m, 4H), 8.73 (d, 1H), 9.00 (t, 1H). UPLC-MS (ESI+): [M+H]⁺=652.

The enantiomers of the racemic material of example 5 were separated by chiral preparative HPLC (method B with Column: Chiralpak ID 5 μm 250×20 mm; Solvent: CO₂/2-propanol 69/31; Flow: 60 mL/min; Temperature: 40° C.; Injection: 0.1-0.2 mL/run, 100 mg/mL DMF; Detection: UV 280 nm) and analytically characterized by HPLC (method E with Column: Chiralpak ID 5 μm 100×4.6 mm; Solvent: CO₂/2-propanol 69/31; Detection: DAD 280 nm):

### Example 5.1

Rₜ=1.24 min (enantiomer 1)

### Example 5.2

Rₜ=5.14 min (enantiomer 2)

### Example 6

### 2-cyclopropyl-N-{[3-fluoro-5-(trifluoromethyl)pyridin-2-yl]methyl}-1-[(3-methoxyphenyl)sulfonyl]-1,2,2′,3′,5′,6′-hexahydrospiro[indole-3,4′-thiopyran]-5-carboxamide 1′,1′-dioxide

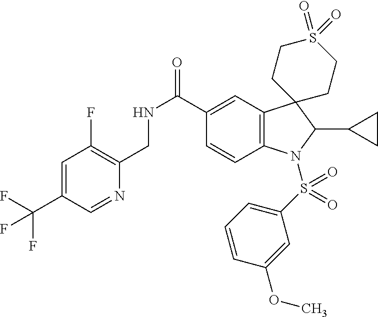 [SMILES: COc1cccc(S(=O)(=O)N2c3ccc(C(=O)NCc4ncc(C(F)(F)F)cc4F)cc3C3(CCS(=O)(=O)CC3)C2C2CC2)c1]

In a slight modification to GP 9.1:100 mg (0.203 mmol) of intermediate F.3 and 81.5 mg (0.305 mmol, 1.5 eq.) 1-[3-fluoro-5-(trifluoromethyl)pyridin-2-yl]methanamine dihydrochloride were reacted with 116 mg (0.305 mmol, 1.5 eq.) HATU in the presence of 128 μL (0.915 mmol, 4.5 eq.) triethylamine in 3.5 mL DMF at rt overnight to yield 117 mg (86%) of the desired amide. The crude product was not further purified. ¹H-NMR (300 MHz, DMSO-d6): Shift [ppm]=0.12-0.24 (m, 1H), 0.34-0.66 (m, 3H), 0.81-1.05 (m, 2H), 1.41 (dt, 1H), 3.64 (dt, 1H), 3.76 (s, 3H), 4.37 (d, 1H), 4.68 (d, 2H) 7.18-7.35 (m, 3H), 7.45 (m, 1H), 7.60 (m, 1H), 7.77-7.89 (m, 2H), 8.28 (m, 1H), 8.80 (s, 1H), 9.13 (t, 1H).). UPLC-MS (ESI+): [M+H]⁺=668.

The enantiomers of the racemic material of example 6 were separated by chiral preparative HPLC (method A with Column: Chiralpak IA 5 μm 250×30 mm; Solvent: hexane/2-propanol 70:30 (v/v); Flow: 40 mL/min; Injection: 0.8 mL/run, 66 mg/mL CH₂C₁₂; Detection: UV 280 nm) and analytically characterized by HPLC (method D with Column: Chiralpak IA 5 μm 150×4.6 mm; Solvent: hexane/2-propanol 70:30 (v/v); Detection: DAD 280 nm):

### Example 6.1

Rₜ=9.68 min (enantiomer 1)

### Example 6.2

Rₜ=16.00 min (enantiomer 2)

### Example 7

### 1-[(4-cyanophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide

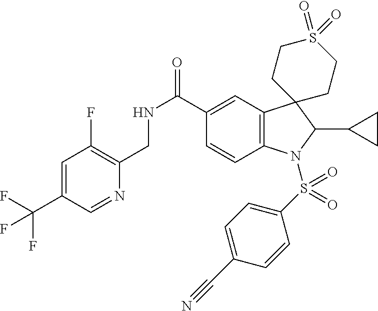 [SMILES: N#Cc1ccc(S(=O)(=O)N2c3ccc(C(=O)NCc4ncc(C(F)(F)F)cc4F)cc3C3(CCS(=O)(=O)CC3)C2C2CC2)cc1]

In a slight modification to GP 9.1:100 mg (0.206 mmol) of intermediate F.7 and 82.3 mg (0.308 mmol, 1.5 eq.) 1-[3-fluoro-5-(trifluoromethyl)pyridin-2-yl]methanamine dihydrochloride were reacted with 117 mg (0.308 mmol, 1.5 eq.) HATU in the presence of 86 μL (0.62 mmol, 3.0 eq.) triethylamine in 2.7 mL DMF at rt overnight. Additionally 25 mg (0.092 mmol, 0.45 eq.) 1-[3-fluoro-5-(trifluoromethyl)pyridin-2-yl]methanamine dihydrochloride and 29 μL (0.21 mmol, 1.0 eq.) triethylamine were added and stirring at rt continued for 22 h. After work-up according to GP 9.1 the crude product was purified by prep. HPLC to yield 63 mg (46%) of the desired amide. ¹H-NMR (300 MHz, DMSO-d6): Shift [ppm]=0.10-0.22 (m, 1H), 0.36-0.67 (m, 3H), 0.79-0.90 (m, 1H), 0.93-1.07 (m, 1H), 1.44 (dt, 1H), 3.61 (dt, 1H), 4.36 (d, 1H), 4.69 (d, 2H), 7.61 (d, 1H), 7.82-8.08 (m, 6H), 8.29 (m, 1H), 8.80 (s, 1H), 9.17 (t, 1H). UPLC-MS (ESI+): [M+H]⁺=663.

The enantiomers of the racemic material of example 7 were separated by chiral preparative HPLC (method C with Column: Chiralpak IC 5 μm 250×30 mm; Solvent: methanol/ethanol 50:50 (v/v); Flow: 50 mL/min; Injection: 0.2 mL/run, 46 mg/mL DMSO; Detection: UV 254 nm) and analytically characterized by HPLC (method D with Column: Chiralpak IC 3 μm 100×4.6 mm; Solvent: methanol/ethanol 50:50 (v/v); Detection: DAD 254 nm):

### Example 7.1

Rₜ=2.93 min (enantiomer 1)

### Example 7.2

Rₜ=6.15 min (enantiomer 2)

### Example 8

### N-{[3-chloro-5-(trifluoromethyl)pyridin-2-yl]methyl}-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide

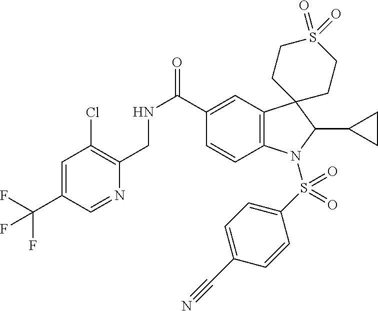 [SMILES: N#Cc1ccc(S(=O)(=O)N2c3ccc(C(=O)NCc4ncc(C(F)(F)F)cc4Cl)cc3C3(CCS(=O)(=O)CC3)C2C2CC2)cc1]

According to GP 9.1 100 mg (0.206 mmol) of intermediate F.7 and 64.9 mg (0.308 mmol, 1.5 eq.) 1-[3-chloro-5-(trifluoromethyl)pyridin-2-yl]methanamine hydrochloride (CAS No. [175277-74-4]) were reacted with 117 mg (0.308 mmol, 1.5 eq.) HATU in the presence of 86 μL (0.62 mmol, 3.0 eq.) triethylamine in 2 mL DMF at rt for 14 h. The obtained crude product was purified by flash chromatography (SiO₂-hexane/ethyl acetate) to give 97 mg (70%) of the desired amide. ¹H-NMR (300 MHz, DMSO-d6): Shift [ppm]=0.13-0.27 (m, 1H), 0.37-0.67 (m, 3H), 0.78-0.91 (m, 1H), 0.93-1.08 (m, 1H), 1.46 (dt, 1H), 3.61 (dt, 1H), 4.37 (d, 1H), 4.74 (d, 2H) 7.62 (d, 1H), 7.84-8.08 (m, 6H), 8.46 (m, 1H), 8.90 (m, 1H), 9.12 (t, 1H). UPLC-MS (ESI+): [M+H]⁺=679/681 (Cl isotope pattern).

The enantiomers of the racemic material of example 8 were separated by chiral preparative HPLC (method A with Column: Chiralpak IC 5 μm 250×30 mm; Solvent: ethanol/methanol 50:50 (v/v); Flow: 40 mL/min; Injection: 1.0 mL/run, 44 mg/mL THF; Detection: UV 280 nm) and analytically characterized by HPLC (method D with Column: Chiralpak IC 3 μm 100×4.6 mm; Solvent: ethanol/methanol 50:50 (v/v); Detection: DAD 280 nm):

### Example 8.1

(2S)—N-{[3-chloro-5-(trifluoromethyl)pyridin-2-yl]methyl}-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide: Rₜ=3.87 min (enantiomer 1)

### Example 8.2

(2R)—N-{[3-chloro-5-(trifluoromethyl)pyridin-2-yl]methyl}-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide: Rₜ=8.31 min (enantiomer 2)

### Example 9

### 1-[(3-cyanophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide

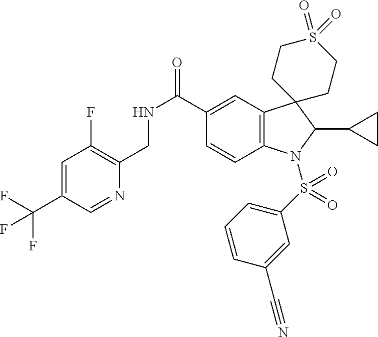 [SMILES: N#Cc1cccc(S(=O)(=O)N2c3ccc(C(=O)NCc4ncc(C(F)(F)F)cc4F)cc3C3(CCS(=O)(=O)CC3)C2C2CC2)c1]

In a slight modification to GP 9.1:115 mg (0.236 mmol) of intermediate F.8 and 80.8 mg (0.303 mmol, 1.28 eq.) 1-[3-fluoro-5-(trifluoromethyl)pyridin-2-yl]methanamine dihydrochloride were reacted with 135 mg (0.354 mmol, 1.5 eq.) HATU in the presence of 99 μL (0.71 mmol, 3.0 eq.) triethylamine in 3 mL DMF at rt for 18 h. Additionally 28 mg (0.11 mmol, 0.45 eq.) 1-[3-fluoro-5-(trifluoromethyl)pyridin-2-yl]methanamine dihydrochloride and 33 μL (0.24 mmol, 1.0 eq.) triethylamine were added and stirring at rt continued for 22 h. After work-up according to GP 9.1 the crude product was purified by flash chromatography (SiO₂-hexane/ethyl acetate) to give 82 mg (52%) of the desired amide. ¹H-NMR (300 MHz, DMSO-d6): Shift [ppm]=0.09-0.15 (m, 1H), 0.40-0.46 (m, 1H), 0.56-0.66 (m, 2H), 0.81-0.86 (m, 1H), 0.96-1.04 (m, 1H), 1.45 (dt, 1H), 3.69 (dt, 1H), 4.35 (d, 1H), 4.69 (d, 2H), 7.63 (d, 1H), 7.74 (t, 1H), 7.84 (m, 1H), 7.89 (m, 1H), 8.01 (d, 1H), 8.12 (d, 1H), 8.28 (m, 1H), 8.42 (s, 1H), 8.80 (s, 1H), 9.15 (t, 1H). UPLC-MS (ESI+): [M+H]⁺=663.

The enantiomers of the racemic material of example 9 were separated by chiral preparative HPLC (method C with Column: Chiralpak IA 5 μm 250×20 mm; Solvent: hexane/2-propanol 70:30 (v/v); Flow: 30 mL/min; Injection: 0.2 mL/run, 47 mg/mL methanol/acetonitrile/DMF 1:1:0.5; Detection: UV 280 nm) and analytically characterized by HPLC (method D with Column: Chiralpak IA 3 μm 100×4.6 mm; Solvent: hexane/2-propanol 70:30 (v/v); Detection: DAD 280 nm):

### Example 9.1

Rₜ=7.30 min (enantiomer 1)

### Example 9.2

Rₜ=11.30 min (enantiomer 2)

### Example 10

### N-{[3-chloro-5-(trifluoromethyl)pyridin-2-yl]methyl}-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide

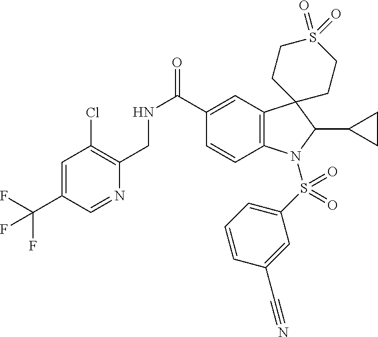 [SMILES: N#Cc1cccc(S(=O)(=O)N2c3ccc(C(=O)NCc4ncc(C(F)(F)F)cc4Cl)cc3C3(CCS(=O)(=O)CC3)C2C2CC2)c1]

In a slight modification to GP 9.1: 115 mg (0.236 mmol) of intermediate F.8 and 74.7 mg (0.354 mmol, 1.5 eq.) 1-[3-chloro-5-(trifluoromethyl)pyridin-2-yl]methanamine hydrochloride (CAS No. [175277-74-4]) were reacted with 135 mg (0.354 mmol, 1.5 eq.) HATU in the presence of 99 μL (0.71 mmol, 3.0 eq.) triethylamine in 3 mL DMF at rt for 18 h. Additionally 25 mg (0.12 mmol, 0.50 eq.) 1-[3-chloro-5-(trifluoromethyl)pyridin-2-yl]methanamine dihydrochloride and 33 μL (0.24 mmol, 1.0 eq.) triethylamine were added and stirring at rt continued for 22 h. The crude reaction mixture was directly submitted to prep. HPLC purification to yield 74 mg (46%) of the desired amide. 1H-NMR (300 MHz, DMSO-d6): Shift [ppm]=0.10-0.17 (m, 1H), 0.40-0.46 (m, 1H), 0.56-0.66 (m, 2H), 0.80-0.86 (m, 1H), 0.97-1.05 (m, 1H), 1.46 (dt, 1H), 3.70 (dt, 1H), 4.35 (d, 1H), 4.74 (d, 2H), 7.64 (d, 1H), 7.75 (t, 1H), 7.86 (m, 1H), 7.90 (m, 1H), 8.02 (m, 1H), 8.13 (d, 1H), 8.43 (s, 1H), 8.46 (m, 1H), 8.90 (s, 1H), 9.12 (t, 1H). UPLC-MS (ESI+): [M+H]⁺=679/681 (Cl isotope pattern).

The enantiomers of the racemic material of example 10 were separated by chiral preparative HPLC (method A with Column: Chiralpak IC 5 μm 250×30 mm; Solvent: ethanol/methanol 50:50 (v/v); Flow: 35 mL/min; Injection: 1.3 mL/run, 25 mg/mL CH₂C₁₂; Detection: UV 280 nm) and analytically characterized by HPLC (method D with Column: Chiralpak IC 3 μm 100×4.6 mm; Solvent: ethanol/methanol 50:50 (v/v); Detection: DAD 280 nm):

### Example 10.1

(2S)—N-{[3-chloro-5-(trifluoromethyl)pyridin-2-yl]methyl}-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide: Rₜ=3.19 min (enantiomer 1)

### Example 10.2

(2R)—N-{[3-chloro-5-(trifluoromethyl)pyridin-2-yl]methyl}-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide: Rₜ=4.09 min (enantiomer 2)

### Example 11

### 2-cyclopropyl-N-{[3-fluoro-5-(trifluoromethyl)pyridin-2-yl]methyl}-1-{[3-(trifluoromethoxy)phenyl]sulfonyl}-1,2,2′,3′,5′,6′-hexahydrospiro[indole-3,4′-thiopyran]-5-carboxamide 1′,1′-dioxide

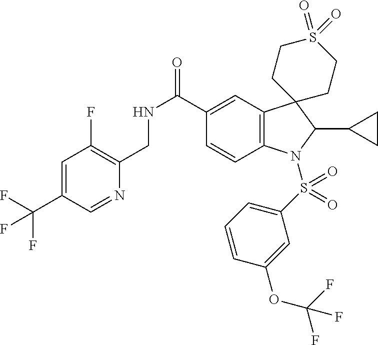 [SMILES: O=C(NCc1ncc(C(F)(F)F)cc1F)c1ccc2c(c1)C1(CCS(=O)(=O)CC1)C(C1CC1)N2S(=O)(=O)c1cccc(OC(F)(F)F)c1]

According to GP 9.1 100 mg (0.183 mmol) of intermediate F.4 and 73.4 mg (0.275 mmol, 1.5 eq.) 1-[3-fluoro-5-(trifluoromethyl)pyridin-2-yl]methanamine dihydrochloride were reacted with 105 mg (0.275 mmol, 1.5 eq.) HATU in the presence of 77 μL (0.55 mmol, 3.0 eq.) triethylamine in 1.6 mL DMF at rt for 22 h to give 136 mg (quant.) of the desired amide. The crude product was not further purified. ¹H-NMR (300 MHz, DMSO-d6): Shift [ppm]=0.18-0.27 (m, 1H), 0.38-0.47 (m, 1H), 0.49-0.66 (m, 2H), 0.81-0.90 (m, 1H), 0.95-1.06 (m, 1H), 1.46 (dt, 1H), 3.65 (dt, 1H), 4.37 (d, 1H), 4.69 (m, 2H), 7.60-7.97 (m, 7H), 8.28 (m, 1H), 8.79 (m, 1H), 9.15 (t, 1H). UPLC-MS (ESI+): [M+H]⁺=722.

The enantiomers of the racemic material of example 11 were separated by chiral preparative HPLC (method B with Column: Chiralpak ID 5 μm 250×20 mm; Solvent: CO₂/2-propanol 75/25; Flow: 80 mL/min; Temperature: 30° C.; Injection: 0.33 mL/run, 32 mg/mL acetonitrile; Detection: UV 254 nm) and analytically characterized by HPLC (method E with Column: Chiralpak ID 5 μm 100×4.6 mm; Solvent: CO₂/2-propanol 75/25; Detection: DAD 254 nm):

### Example 11.1

Rₜ=1.93 min (enantiomer 1)

### Example 11.2

Rₜ=3.16 min (enantiomer 2)

### Example 12

### N-{[3-chloro-5-(trifluoromethyl)pyridin-2-yl]methyl}-2-cyclopropyl-1-{[3-(trifluoromethoxy)phenyl]sulfonyl}-1,2,2′,3′,5′,6′-hexahydrospiro[indole-3,4′-thiopyran]-5-carboxamide 1′,1′-dioxide

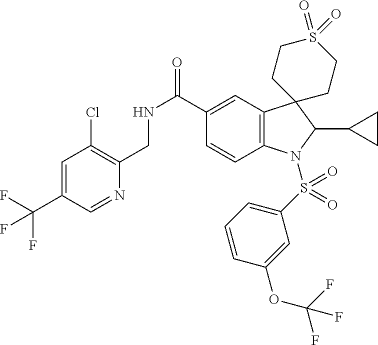 [SMILES: O=C(NCc1ncc(C(F)(F)F)cc1Cl)c1ccc2c(c1)C1(CCS(=O)(=O)CC1)C(C1CC1)N2S(=O)(=O)c1cccc(OC(F)(F)F)c1]

According to GP 9.1 100 mg (0.183 mmol) of intermediate F.4 and 67.9 mg (0.275 mmol, 1.5 eq.) 1-[3-chloro-5-(trifluoromethyl)pyridin-2-yl]methanamine hydrochloride (CAS No. [175277-74-4]) were reacted with 105 mg (0.275 mmol, 1.5 eq.) HATU in the presence of 77 μL (0.55 mmol, 3.0 eq.) triethylamine in 1.6 mL DMF at rt for 22 h to give 90 mg (67%) of the desired amide. The crude product was not further purified. ¹H-NMR (300 MHz, DMSO-d6): Shift [ppm]=0.20-0.30 (m, 1H), 0.37-0.48 (m, 1H), 0.49-0.66 (m, 2H), 0.80-0.90 (m, 1H), 0.96-1.07 (m, 1H), 1.47 (dt, 1H), 3.66 (dt, 1H), 4.38 (d, 1H), 4.74 (m, 2H), 7.60-7.98 (m, 7H), 8.46 (m, 1H), 8.90 (m, 1H), 9.12 (t, 1H). UPLC-MS (ESI+): [M+H]⁺=738/740 (Cl isotope pattern).

The enantiomers of the racemic material of example 12 were separated by chiral preparative HPLC (method B with Column: Chiralpak ID 5 μm 250×20 mm; Solvent: CO₂/2-propanol 75/25; Flow: 80 mL/min; Temperature: 30° C.; Injection: 0.2 mL/run, 39 mg/mL acetonitrile/DMSO 10:1; Detection: UV 254 nm) and analytically characterized by HPLC (method E with Column: Chiralpak ID 5 μm 100×4.6 mm; Solvent: CO₂/2-propanol 75/25; Detection: DAD 254 nm):

### Example 12.1

Rₜ=2.29 min (enantiomer 1)

### Example 12.2

Rₜ=2.91 min (enantiomer 2)

### Example 13

### 2-cyclopropyl-1-{[3-(difluoromethoxy)phenyl]sulfonyl}-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide

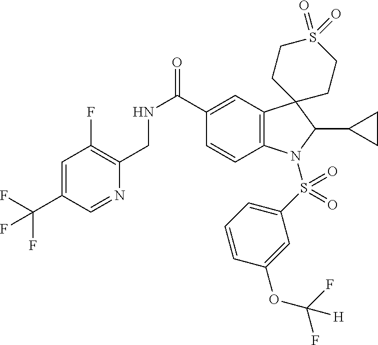 [SMILES: O=C(NCc1ncc(C(F)(F)F)cc1F)c1ccc2c(c1)C1(CCS(=O)(=O)CC1)C(C1CC1)N2S(=O)(=O)c1cccc(OC(F)F)c1]

According to GP 9.1 100 mg (0.190 mmol) of intermediate F.5 and 75.9 mg (0.284 mmol, 1.5 eq.) 1-[3-fluoro-5-(trifluoromethyl)pyridin-2-yl]methanamine dihydrochloride were reacted with 108 mg (0.284 mmol, 1.5 eq.) HATU in the presence of 79 μL (0.57 mmol, 3.0 eq.) triethylamine in 1.6 mL DMF at rt for 22 h to give 99 mg (74%) of the desired amide. The crude product was not further purified. ¹H-NMR (300 MHz, DMSO-d6): Shift [ppm]=0.18-0.27 (m, 1H), 0.37-0.47 (m, 1H), 0.49-0.65 (m, 2H), 0.82-0.91 (m, 1H), 0.94-1.05 (m, 1H), 1.45 (dt, 1H), 3.65 (dt, 1H), 4.36 (d, 1H), 4.69 (m, 2H), 7.31 (tr, 1H), 7.46-7.66 (m, 5H), 7.82-7.90 (m, 2H), 8.28 (m, 1H), 8.80 (m, 1H), 9.15 (t, 1H). UPLC-MS (ESI+): [M+H]⁺=704.

The enantiomers of the racemic material of example 13 were separated by chiral preparative HPLC (method B with Column: Chiralpak ID 5 μm 250×20 mm; Solvent: CO₂/2-propanol 70/30; Flow: 80 mL/min; Temperature: 30° C.; Injection: 0.5 mL/run, 37 mg/mL methanol/DMSO 4:1; Detection: UV 254 nm) and analytically characterized by HPLC (method E with Column: Chiralpak ID 5 μm 100×4.6 mm; Solvent: CO₂/2-propanol 70/30; Detection: DAD 254 nm):

### Example 13.1

Rₜ=1.96 min (enantiomer 1)

### Example 13.2

Rₜ=3.55 min (enantiomer 2)

### Example 14

N-{[3-chloro-5-(trifluoromethyl)pyridin-2-yl]methyl}-2-cyclopropyl-1-{[3-(difluoromethoxy)phenyl]sulfonyl}-1,2,2′,3′,5′,6′-hexahydrospiro[indole-3,4′-thiopyran]-5-carboxamide 1′,1′-dioxide

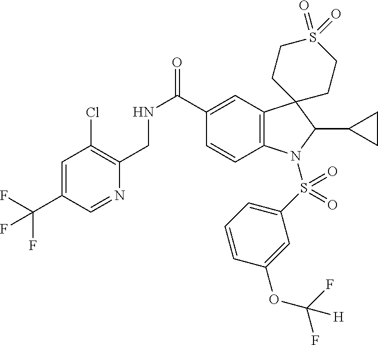 [SMILES: O=C(NCc1ncc(C(F)(F)F)cc1Cl)c1ccc2c(c1)C1(CCS(=O)(=O)CC1)C(C1CC1)N2S(=O)(=O)c1cccc(OC(F)F)c1]

According to GP 9.1 100 mg (0.190 mmol) of intermediate F.5 and 70.2 mg (0.284 mmol, 1.5 eq.) 1-[3-chloro-5-(trifluoromethyl)pyridin-2-yl]methanamine hydrochloride (CAS No. [175277-74-4]) were reacted with 108 mg (0.284 mmol, 1.5 eq.) HATU in the presence of 79 μL (0.57 mmol, 3.0 eq.) triethylamine in 1.6 mL DMF at rt for 22 h to give 119 mg (87%) of the desired amide. The crude product was not further purified. ¹H-NMR (300 MHz, DMSO-d6): Shift [ppm]=0.19-0.28 (m, 1H), 0.38-0.46 (m, 1H), 0.48-0.65 (m, 2H), 0.81-0.91 (m, 1H), 0.94-1.05 (m, 1H), 1.46 (dt, 1H), 3.65 (dt, 1H), 4.36 (d, 1H), 4.74 (m, 2H), 7.32 (t, 1H), 7.45-7.67 (m, 5H), 7.82-7.96 (m, 2H), 8.46 (m, 1H), 8.90 (m, 1H), 9.11 (t, 1H). UPLC-MS (ESI+): [M+H]⁺=720/722 (Cl isotope pattern).

The enantiomers of the racemic material of example 14 were separated by chiral preparative HPLC (method B with Column: Chiralpak ID 5 μm 250×20 mm; Solvent: CO₂/2-propanol 75/25; Flow: 60 mL/min; Temperature: 40° C.; Injection: 0.5 mL/run, 46 mg/mL CH₂Cl₂/CHCl₃ 1:1; Detection: UV 254 nm) and analytically characterized by HPLC (method E with Column: Chiralpak ID 5 μm 100×4.6 mm; Solvent: CO₂/2-propanol 75/25; Detection: DAD 254 nm):

### Example 14.1

Rₜ=3.53 min (enantiomer 1)

### Example 14.2

Rₜ=4.87 min (enantiomer 2)

### Example 15

### 2-cyclopropyl-1-{[4-(difluoromethoxy)phenyl]sulfonyl}-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide

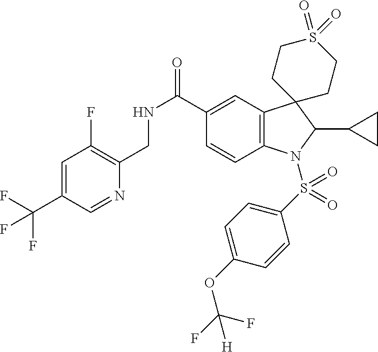 [SMILES: O=C(NCc1ncc(C(F)(F)F)cc1F)c1ccc2c(c1)C1(CCS(=O)(=O)CC1)C(C1CC1)N2S(=O)(=O)c1ccc(OC(F)F)cc1]

In a slight modification to GP 9.1: 120 mg (0.227 mmol) of intermediate F.6 and 91.1 mg (0.341 mmol, 1.5 eq.) 1-[3-fluoro-5-(trifluoromethyl)pyridin-2-yl]methanamine dihydrochloride were reacted with 130 mg (0.341 mmol, 1.5 eq.) HATU in the presence of 143 μL (1.02 mmol, 4.5 eq.) triethylamine in 2.2 mL DMF at rt overnight. The crude reaction mixture was directly submitted to prep. HPLC purification to yield 100 mg (62%) of the desired amide. ¹H-NMR (300 MHz, DMSO-d6): Shift [ppm]=0.13-0.25 (m, 1H), 0.34-0.66 (m, 3H), 0.79-0.91 (m, 1H), 0.92-1.07 (m, 1H), 1.44 (dt, 1H), 3.61 (dt, 1H), 4.35 (d, 1H), 4.69 (m, 2H), 7.31 (d, 2H), 7.35 (t, 1H), 7.59 (d, 1H), 7.78-7.92 (m, 4H), 8.28 (m, 1H), 8.79 (m, 1H), 9.13 (t, 1H). UPLC-MS (ESI+): [M+H]⁺=704.

The enantiomers of the racemic material of example 15 were separated by chiral preparative HPLC (method B with Column: Chiralpak ID 5 μm 250×20 mm; Solvent: CO₂/2-propanol 65/35; Flow: 80 mL/min; Temperature: 40° C.; Injection: 0.2 or 0.4 mL/run, 60 mg/mL CH₂Cl₂/CHCl₃/DMF 2:1:1; Detection: UV 254 nm) and analytically characterized by HPLC (method F):

### Example 15.1

Rₜ=1.13 min (enantiomer 1)

### Example 15.2

Rₜ=2.28 min (enantiomer 2)

### Example 16

### N-{[3-chloro-5-(trifluoromethyl)pyridin-2-yl]methyl}-2-cyclopropyl-1-{[4-(difluoromethoxy)phenyl]sulfonyl}-1,2,2′,3′,5′,6′-hexahydrospiro[indole-3,4′-thiopyran]-5-carboxamide 1′,1′-dioxide

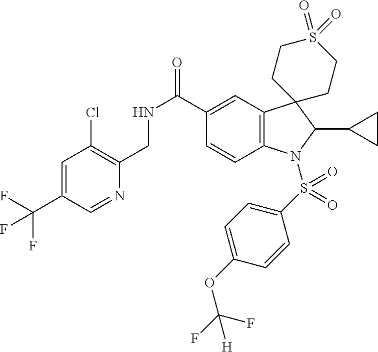 [SMILES: O=C(NCc1ncc(C(F)(F)F)cc1Cl)c1ccc2c(c1)C1(CCS(=O)(=O)CC1)C(C1CC1)N2S(=O)(=O)c1ccc(OC(F)F)cc1]

According to GP 9.1 120 mg (0.227 mmol) of intermediate F.6 and 84.3 mg (0.341 mmol, 1.5 eq.) 1-[3-chloro-5-(trifluoromethyl)pyridin-2-yl]methanamine hydrochloride (CAS No. [175277-74-4]) were reacted with 130 mg (0.341 mmol, 1.5 eq.) HATU in the presence of 95 μL (0.682 mmol, 3.0 eq.) triethylamine in 2.2 mL DMF at rt overnight. The crude reaction mixture was directly submitted to prep. HPLC purification to yield 100 mg (61%) of the desired amide. ¹H-NMR (300 MHz, DMSO-d6): Shift [ppm]=0.15-0.28 (m, 1H), 0.36-0.67 (m, 3H), 0.81-0.92 (m, 1H), 0.92-1.08 (m, 1H), 1.45 (dt, 1H), 3.61 (dt, 1H), 4.36 (d, 1H), 4.74 (m, 2H), 7.31 (d, 2H), 7.36 (t, 1H), 7.60 (d, 1H), 7.81-7.93 (m, 4H), 8.46 (m, 1H), 8.90 (m, 1H), 9.10 (t, 1H). UPLC-MS (ESI+): [M+H]⁺=720/722 (Cl isotope pattern).

The enantiomers of the racemic material of example 16 were separated by chiral preparative HPLC (method B with Column: Chiralpak ID 5 μm 250×20 mm; Solvent: CO₂/2-propanol 70/30; Flow: 80 mL/min; Temperature: 40° C.; Injection: 0.1 mL/run, 89 mg/mL acetone/ethyl acetate 1:1; Detection: UV 254 nm) and analytically characterized by HPLC (method E with Column: Chiralpak ID 5 μm 100×4.6 mm; Solvent: CO₂/2-propanol 70/30; Detection: DAD 254 nm):

### Example 16.1

Rₜ=2.22 min (enantiomer 1)

### Example 16.2

Rₜ=3.39 min (enantiomer 2)

### Example 17

### 1-[(4-carbamoylphenyl)sulfonyl]-N-{[3-chloro-5-(trifluoromethyl)pyridin-2-yl]methyl}-2-cyclopropyl-1,2,2′,3′,5′,6′-hexahydrospiro[indole-3,4′-thiopyran]-5-carboxamide 1′,1′-dioxide

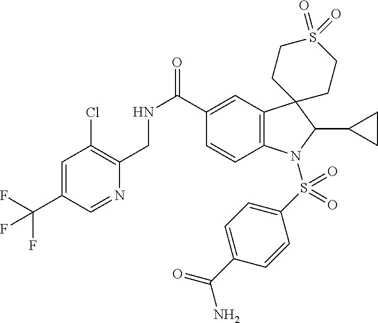 [SMILES: NC(=O)c1ccc(S(=O)(=O)N2c3ccc(C(=O)NCc4ncc(C(F)(F)F)cc4Cl)cc3C3(CCS(=O)(=O)CC3)C2C2CC2)cc1]

In a slight modification to GP 9.1: 86 mg (0.17 mmol) of intermediate F.9 were dissolved in 0.4 mL DMF and treated with a solution of 78 mg (0.21 mmol, 1.2 eq.) HATU in 0.2 mL DMF, 88 μL (0.63 mmol, 3.7 eq.) triethylamine and a solution of 51 mg (0.21 mmol, 1.2 eq.) 1-[3-chloro-5-(trifluoromethyl)pyridin-2-yl]methanamine hydrochloride (CAS No. [175277-74-4]) in 0.4 mL DMF and stirred at rt overnight. The reaction mixture was taken up with EtOAc, the organic phase washed with water, dried with sodium sulfate and the solvents removed in vacuo. The crude product was purified by prep. HPLC purification to yield 68 mg (57%) of the desired amide. ¹H-NMR (400 MHz, DMSO-d6): Shift [ppm]=0.14-0.19 (m, 1H), 0.39-0.46 (m, 1H), 0.50-0.56 (m, 1H), 0.58-0.64 (m, 1H), 0.84-0.90 (m, 1H), 0.97-1.06 (m, 1H), 1.41 (dt, 1H), 2.52-2.61 (m, 3H), 3.20-3.23 (m, 2H), 3.61-3.68 (m, 1H), 4.38 (d, 1H), 4.68-4.78 (m, 2H), 7.60-7.63 (m, 2H), 7.84 (d, 1H), 7.87-7.95 (m, 5H), 8.11 (br. s., 1H), 8.46 (d, 1H), 8.90 (d, 1H), 9.10 (t, 1H). UPLC-MS (ESI+): [M+H]⁺=697/699 (Cl isotope pattern).

The enantiomers of the racemic material of example 17 were separated by chiral preparative HPLC (method C with Column: Chiralpak IC 5 μm 250×20 mm; Solvent: methanol; Flow: 30 mL/min; Injection: 50-75 μL/run, 47 mg/mL methanol/CH₂Cl₂/DMF 1:1:1; Detection: UV 254 nm) and analytically characterized by HPLC (method D with Column: Chiralpak IC 3 μm 100×4.6 mm; Solvent: methanol; Detection: DAD 254 nm):

### Example 17.1

Rₜ=2.60 min (enantiomer 1)

### Example 17.2

Rₜ=2.90 min (enantiomer 2)

### Example 18

### 1-[(4-chlorophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide

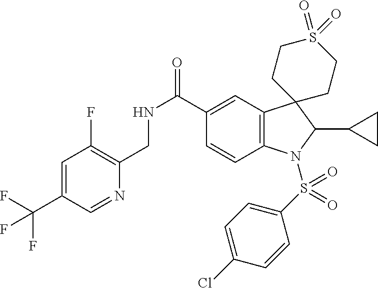 [SMILES: O=C(NCc1ncc(C(F)(F)F)cc1F)c1ccc2c(c1)C1(CCS(=O)(=O)CC1)C(C1CC1)N2S(=O)(=O)c1ccc(Cl)cc1]

In a slight modification to GP 9.1: 130 mg (0.262 mmol) of intermediate F.10 and 105 mg (0.393 mmol, 1.5 eq.) 1-[3-fluoro-5-(trifluoromethyl)pyridin-2-yl]methanamine dihydrochloride were reacted with 149 mg (0.393 mmol, 1.5 eq.) HATU in the presence of 164 μL (1.18 mmol, 4.5 eq.) triethylamine in 2.5 mL DMF at rt overnight. The crude reaction mixture was directly submitted to prep. HPLC purification to yield 120 mg (68%) of the desired amide. ¹H-NMR (300 MHz, DMSO-d6): Shift [ppm]=0.28-0.66 (m, 4H), 0.79-0.90 (m, 1H), 0.92-1.08 (m, 1H), 1.49 (dt, 1H), 3.65 (dt, 1H), 4.37 (d, 1H), 4.69 (m, 2H), 7.53-7.69 (m, 3H), 7.78-7.95 (m, 4H), 8.28 (m, 1H), 8.79 (s, 1H), 9.14 (t, 1H). UPLC-MS (ESI+): [M+H]⁺=672/674 (Cl isotope pattern).

The enantiomers of the racemic material of example 18 were separated by chiral preparative HPLC (method C with Column: Chiralpak IC 5 μm 250×30 mm; Solvent: ethanol/methanol 50:50 (v/v); Flow: 30 mL/min; Injection: 1.3 mL/run, 29 mg/mL CH₂C₁₂; Detection: UV 280 nm) and analytically characterized by HPLC (method D with Column: Chiralpak IC 3 μm 100×4.6 mm; Solvent: ethanol/methanol 50:50 (v/v); Detection: DAD 280 nm):

### Example 18.1

Rₜ=2.38 min (enantiomer 1)

### Example 18.2

Rₜ=2.78 min (enantiomer 2)

### Example 19

### 1-[(4-chlorophenyl)sulfonyl]-N-{[3-chloro-5-(trifluoromethyl)pyridin-2-yl]methyl}-2-cyclopropyl-1,2,2′,3′,5′,6′-hexahydrospiro[indole-3,4′-thiopyran]-5-carboxamide 1′,1′-dioxide

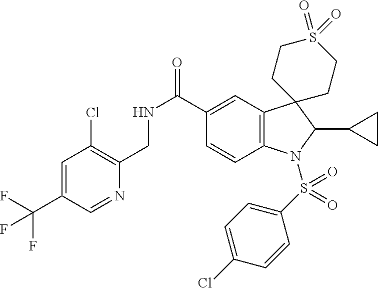 [SMILES: O=C(NCc1ncc(C(F)(F)F)cc1Cl)c1ccc2c(c1)C1(CCS(=O)(=O)CC1)C(C1CC1)N2S(=O)(=O)c1ccc(Cl)cc1]

According to GP 9.1 130 mg (0.262 mmol) of intermediate F.10 and 97 mg (0.39 mmol, 1.5 eq.) 1-[3-chloro-5-(trifluoromethyl)pyridin-2-yl]methanamine hydrochloride (CAS No. [175277-74-4]) were reacted with 149 mg (0.393 mmol, 1.5 eq.) HATU in the presence of 110 μL (0.786 mmol, 3.0 eq.) triethylamine in 2.5 mL DMF at rt overnight. The crude reaction mixture was directly submitted to prep. HPLC purification to yield 120 mg (66%) of the desired amide. ¹H-NMR (300 MHz, DMSO-d6): Shift [ppm]=0.29-0.66 (m, 4H), 0.79-0.92 (m, 1H), 0.92-1.08 (m, 1H), 1.50 (dt, 1H), 3.66 (dt, 1H), 4.74 (d, 1H), 7.54-7.68 (m, 3H), 7.79-7.95 (m, 4H), 8.45 (m, 1H), 8.90 (m, 1H), 9.11 (t, 1H). UPLC-MS (ESI+): [M+H]⁺=688/690 (Cl isotope pattern).

The enantiomers of the racemic material of example 19 were separated by chiral preparative HPLC (method C with Column: Chiralpak IB 5 μm 250×30 mm; Solvent: hexane/ethanol 70:30 (v/v); Flow: 45 mL/min; Injection: 0.5 mL/run, 75 mg/mL CH₂Cl₂; Detection: UV 280 nm) and analytically characterized by HPLC (method D with Column: Chiralpak IB 3 μm 100×4.6 mm; Solvent: hexane/ethanol 70:30 (v/v); Detection: DAD 280 nm):

### Example 19.1

Rₜ=5.24 min (enantiomer 1)

### Example 19.2

Rₜ=6.16 min (enantiomer 2)

### Example 20

### N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide

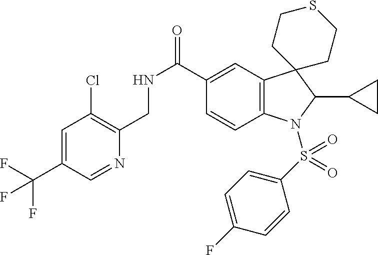 [SMILES: O=C(NCc1ncc(C(F)(F)F)cc1Cl)c1ccc2c(c1)C1(CCSCC1)C(C1CC1)N2S(=O)(=O)c1ccc(F)cc1]

According to GP 9.1 200 mg (0.447 mmol) of intermediate F.11 and 166 mg (0.670 mmol, 1.5 eq.) 1-[3-chloro-5-(trifluoromethyl)pyridin-2-yl]methanamine hydrochloride (CAS No. [175277-74-4]) were reacted with 255 mg (0.670 mmol, 1.5 eq.) HATU in the presence of 187 μL (1.34 mmol, 3.00 eq.) triethylamine in 5 mL DMF at rt overnight. Deviating from GP 9.1 the crude reaction mixture was acidified with 2 M HCl_{aq} and extracted with ethyl acetate. The organic phase was washed with water (three times) and brine, dried with sodium sulfate and the solvents removed in vacuo. The crude product was purified by prep. HPLC purification to yield 86 mg (30%) of the desired amide. ¹H-NMR (400 MHz, DMSO-d6): Shift [ppm]=0.29-0.32 (m, 1H), 0.36-0.43 (m, 1H), 0.44-0.50 (m, 1H), 0.58-0.65 (m, 1H), 0.74-0.80 (m, 1H), 0.93-1.07 (m, 2H), 2.01-2.04 (m, 1H), 2.13-2.20 (m, 1H), 2.37-2.41 (m, 1H), 2.66-2.69 (m, 1H), 2.79-2.92 (m, 2H), 4.08 (d, 1H), 4.73 (d, 2H), 7.37-7.42 (m, 2H), 7.57 (d, 1H), 7.79 (d, 1H), 7.84 (dd, 1H), 7.87-7.91 (m, 2H), 8.46 (d, 1H), 8.90 (d, 1H), 9.03 (t, 1H). UPLC-MS (ESI+): [M+H]⁺=640/642 (Cl isotope pattern).

The enantiomers of the racemic material of example 20 were separated by chiral preparative HPLC (method C with Column: Chiralpak IC 5 μm 250×20 mm; Solvent: hexane/2-propanol 70:30 (v/v); Flow: 20 mL/min; Injection: 0.5 mL/run, 34 mg/mL CH₂Cl₂/methanol; Detection: UV 254 nm) and analytically characterized by HPLC (method D with Column: Chiralpak IC 3 μm 100×4.6 mm; Solvent: hexane/2-propanol 70:30 (v/v); Detection: DAD 254 nm):

### Example 20.1

Rₜ=4.20 min (enantiomer 1)

### Example 20.2

Rₜ=4.94 min (enantiomer 2)

### Biological Assays

### 1. Materials

Buserelin was purchased from Welding (Frankfurt/Main, Germany) or USbiological (#B8995, Swampscott, USA) for IP-One HTRF® assays and LHRH from Sigma-Aldrich® (Munich, Germany). Labelled cells, Tag-Lite buffer, labelled and unlabelled GnRHR binding peptide for Tag-lite® binding assay was purchased by Cisbio Bioassays (Bagnols-sur-Ceze Cedex, France). The radio labelling was performed in the Department of Isotope Chemistry of Bayer Pharma AG (Berlin, Germany) by the iodogen method using [¹²⁵I]sodium iodide (2000 Ci/mmol; PerkinElmer Life and Analytical Sciences, USA) yielding [¹²⁵I]monoiodo-buserelin. The radio-tracer was purified by reversed phase HPLC on a Spherisorb ODS 11 column (250×4 mm, particle size 3 μm) by elution with acetonitrile/water (34:66) containing 39 mM trifluoracetic acid at a flow rate of 1 mL/min.

The retention time of [¹²⁵I]monoiodo-buserelin was approximately 17 min. All other chemicals were obtained from commercial sources at the highest purity grade available.

### 2. Methods

### 2.1. Receptor Binding Assay Using Radiolabelled Buserelin

Binding studies for competition curves were run in triplicate samples in 96 well polypropylene microtiter plates (Nunc, N.J., USA). One assay sample contained 70 μl of 300,000 cells for CHO cells stably transfected with the human GnRH receptor, 20 μl of ¹²⁵I-labelled buserelin (100,000 cpm per sample for competition curves) and 10 μl of assay buffer or test compound solution. Test compounds were dissolved in DMSO. Cetrorelix was dissolved in 0.1 M hydrochloric acid. Serial dilutions (5×10⁻⁶ M to 5×10⁻¹² M) were prepared in assay buffer (DMEM or DMEM/Ham's F12 medium, 10 mM Hepes buffer pH 7.5, 0.5% BSA). Nonspecific binding was determined in presence of excess unlabelled buserelin (10⁻⁵ M). Test samples were incubated for 60 min at room temperature. Bound and free ligand were separated by filtration over Unifilter GF/C filter microtiter plates (PerkinElmer, CT, USA) by applying negative pressure and washing twice with 200 mL of 0.02 M Tris/hydrochloric acid, pH 7.4. The filter plates were soaked with 0.3% polyethylenimine (Serve; Heidelberg, Germany) for 30 min prior to use in order to reduce nonspecific binding. The radioactivity retained by the filters was determined in a TopCount NXT HTS (PerkinElmer, CT, USA) using 20 μl/well MicroScint40 scintillator cocktail (PerkinElmer, CT, USA). Competition curves were obtained by plotting the measured radioactivity against the respective test compound concentration by using an in-house software.

### 2.2. Tag-Lite® Receptor Binding Assay

This binding assay is based on the fluorescence resonance energy transfer between fluorescence donor labelled human GnRHR and a green-labelled GnRHR binding peptide. Compounds interfering with the ligand binding side of the human GnRHR will replace the labelled peptide resulting in a signal decrease. The assay principle was established by Cisbio Bioassays (Bagnols-sur-Cèze Cedex, France) and further details are available on their homepage.

The assay procedure was further optimized for use in-house with reduced assay volumes. Frozen Hek293 cells, transiently transfected with human GnRHR and Terbium-labelling of the receptor, were supplied by Cisbio Bioassays as well as Tag-Lite buffer and green-labelled GnRHR binding peptide. Cells were thawed and transferred to cold Tag-Lite buffer. A volume of 8 μl of this cell suspension were added to 100 nl of a 160-fold concentrated solution of the test compound in DMSO pre-dispensed in a well of a white low-volume 384-well microtiter plate (Greiner Bio-One, Frickenhausen, Germany). The mixture was incubated for 5 min at room temperature. In the next step either 4 μl Tag-Lite buffer or as control 4 μl of an exceeding amount unlabelled binding peptide in Tag-Lite buffer were transferred to the mixture. The green-labelled GnRHR binding peptide was added in a final step at EC₅₀ in a volume of 4 μl Tag-Lite buffer. After an incubation of 1 h at room temperature plates were measured in a microplate reader, e.g. a PHERAstar (BMG Labtechnologies, Offenburg, Germany) by using a specific optic module.

A ratio from the fluorescence emissions at 520 nm (green fluorescence) and at 490 nm (background signal of Terbium-labelled GnRHR) was calculated and the data were normalized (reaction without test compound=0% inhibition of binding of green-labelled peptide; reaction without test compound with exceeding amount unlabelled binding peptide=100% inhibition of binding of green-labelled peptide). On the same microtiter plate, compounds were tested at 10 different concentrations in the range of 12.5 μM to 0.64 nM (12.5 μM, 4.2 μM, 1.4 μM, 0.46 μM, 0.15 μM, 51 nM, 17 nM, 5.7 nM, 1.9 nM and 0.64 nM; dilution series prepared before the assay at the level of the 160-fold conc. stock solutions by serial 1:3 dilutions in 100% DMSO) in duplicate values for each concentration. By using an in-house software, the IC₅₀ values were calculated by a 4 parameter fit.

TABLE 1
Potency in receptor binding assay using TAG-LITE ®
technology; the potency is given as IC₅₀ [μM[.

| Example | Potency [μM] |
|---|---|
| 1.1 | 0.23 |
| 3.1 | 0.0167 |
| 3.2 | 0.748 |
| 8.0 | 0.0217 |
| 10.1 | 0.0103 |
| 14.0 | 0.0787 |
| 17.2 | 0.0817 |

### 2.3. IP-One HTRF® Assay

By using homogenous time-resolved fluorescence resonance energy transfer (HTRF), the generation of one component of the GnRH-R signalling cascade can be measured. After stimulation of CHO cells stably expressing human GnRH receptor (established by Prof. Thomas Gudermann, currently University of Marburg, Germany; supplied as frozen cell aliquots by Cell Culture Services, Hamburg, Germany) with the EC₈₀ of the GnRH agonist buserelin, Gq protein-coupled receptor signalling cascade is activated resulting in PLC-dependent cleavage of PIP2 to Inositol-1,4,5-triphosphate (IP3) and Diacylglycerol. The second messenger IP3 is degraded intracellularly to myo-inositol. Inhibition of the final degradation step from Inositol-1-phosphate (IP1) to myo-inositol by addition of lithium chloride leads to accumulation of IP1 in the cells. In cell lysates, IP1 can be detected via an antibody-based HTRF detection technology, where IP1 can displace the FRET acceptor IP1-d2 from binding by Terbium-labelled anti-IP1 antibody as donor resulting in a signal decrease. Compounds were tested for their capability of inhibiting GnRH-R activation by buserelin. For all IP-One HTRF® assays reagents of Cisbio Bioassays (IP-One Tb Jumbo kit, #62IPAPEJ; Cisbio Bioassays, Bagnols sur Cèze Cedex, France) were used.

For the assay, frozen cell aliquots were thawed and a cell suspension (3.33×10⁶ cells/mL) containing IP1-d2 (dilution 1:40) was prepared and incubated at 37° C. After 1 h 3 μl of the cell suspension were added to 50 nl of a 100-fold concentrated solution of the test compound in DMSO pre-dispensed in a well of a white low-volume 384-well microtiter plate (Greiner Bio-One, Frickenhausen, Germany). The mixture was incubated for 20 min at 22° C. to allow for pre-binding of the test compound to the GnRH-R. The receptor signaling cascade was stimulated by addition of 2 μl buserelin or LHRH (at EC₅₀ or EC₈₀) in stimulation buffer (10 mM Hepes pH 7.4, 1 mM CaCl2, 0.5 mM MgCl2, 4.2 mM KCl, 146 mM NaCl, 5.5 mM a-D-Glucose, 0.05% BSA, 125 mM LiCl (final assay concentration 50 mM) in aqua dest.). Plates were incubated for 1 h at 37° C. and 5% carbon dioxide before the cells were lysed by adding 3 μl Terbium-labelled anti-IP1 antibody (1:40) diluted in Conjugate & Lysis buffer as supplied with the kit. After an incubation for 1 h at 22° C. to enable complete cell lysis and antibody binding to free IP1 or IP1-d2, plates were measured in an HTRF reader, e.g. a RUBYstar, PHERAstar (both BMG Labtechnologies, Offenburg, Germany) or a Viewlux (PerkinElmer LAS, Rodgau-Jügesheim, Germany).

From the fluorescence emissions at 665 nm (FRET) and at 620 nm (background signal of Terbium-antibody), the ratio (emission at 665 nm divided by emission at 620 nm) was calculated and the data were normalized (reaction without test compound=0% inhibition; all other assay components except agonist=100% inhibition). On the same microtiter plate, compounds were tested at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM; dilution series prepared before the assay at the level of the 100-fold conc. stock solutions by serial 1:3 dilutions in 100% DMSO) in duplicate values for each concentration. By using an in-house software, the IC₅₀ values were calculated by a 4 parameter fit.

The data reveal that the compounds of the present invention have antagonist activities on the human GnRH receptor.

Within the meaning of the present invention the antagonist activity is reflected by the ability of a compound of the invention to antagonize human GnRH receptor stimulation in IP-One HTRF® assay at least three times the standard deviation over the background level.

TABLE 2
Potency in IP-One HTRF ® assay with buserelin (at EC₈₀)
stimulation; the potency is given as IC₅₀ [μM].

| Example | Potency [μM] |
|---|---|
| 1 | 0.420 |
| 1.1 | 0.093 |
| 1.2 | 6.99 |
| 2 | 0.027 |
| 3 | 0.053 |
| 3.1 | 0.021 |
| 3.2 | 2.43 |
| 4 | 0.069 |
| 4.1 | 0.0033 |
| 4.2 | 4.08 |
| 5 | 0.864 |
| 5.1 | 0.606 |
| 5.2 | 4.25 |
| 6 | 0.231 |
| 6.1 | 0.110 |
| 6.2 | 3.26 |
| 7 | 0.069 |
| 7.1 | >20.0 |
| 7.2 | 0.057 |
| 8 | 0.022 |
| 8.1 | 0.010 |
| 8.2 | 3.56 |
| 9 | 0.311 |
| 9.1 | 0.107 |
| 9.2 | 2.31 |
| 10 | 0.029 |
| 10.1 | 0.014 |
| 10.2 | 1.97 |
| 11 | 0.760 |
| 11.1 | 0.262 |
| 11.2 | >20.0 |
| 12 | 0.219 |
| 12.1 | 0.068 |
| 12.2 | 4.21 |
| 13 | 0.423 |
| 13.1 | 0.122 |
| 13.2 | >20.0 |
| 14 | 0.082 |
| 14.1 | 0.022 |
| 14.2 | 3.95 |
| 15 | 0.112 |
| 15.1 | 0.045 |
| 15.2 | >20.0 |
| 16 | 0.037 |
| 16.1 | 0.024 |
| 16.2 | 0.879 |
| 17 | 0.260 |
| 17.1 | >20.0 |
| 17.2 | 0.122 |
| 18 | 0.065 |
| 18.1 | 0.031 |
| 18.2 | 1.53 |
| 19 | 0.031 |
| 19.1 | 9.35 |
| 19.2 | 0.0055 |

### 2.4 In Vivo Pharmacokinetics in Rats

For in vivo pharmacokinetic experiments test compounds were administered to male Wistar rats intravenously at a dose of 0.5 mg/kg and intragastral at a dose of 2 mg/kg formulated as solutions using solubilizers such as PEG400 or Solutol in well-tolerated amounts.

For pharmacokinetics after intravenous administration test compounds were given as i.v. bolus and blood samples were taken at 2 min, 8 min, 15 min, 30 min, 45 min, 1 h, 2 h, 4 h, 7 h and 24 h after dosing. Depending on the expected half-life additional samples were taken at later time points (e.g. 48 h, 72 h). For pharmacokinetics after intragastral administration test compounds were given intragastral to fasted rats and blood samples were taken at 8 min, 15 min, 30 min, 45 min, 1 h, 2 h, 4 h, 7 h and 24 h after dosing. Depending on the expected half-life additional samples were taken at later time points (e.g. 48 h, 72 h). Blood was collected into Lithium-Heparintubes (Monovetten®, Sarstedt) and centrifuged for 15 min at 3000 rpm. An aliquot of 100 μL from the supernatant (plasma) was taken and precipitated by addition of 400 μL cold acetonitril and frozen at −20° C. over night. Samples were subsequently thawed and centrifuged at 3000 rpm, 4° C. for 20 minutes. Aliquots of the supernatants were taken for analytical testing using an Agilent 1200 HPLC-system with LCMS/MS detection. PK parameters were calculated by non-compartmental analysis using a PK calculation software.

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. PK parameters calculated from concentration time profiles after i.g.: Cmax: Maximal plasma concentration (in mg/L); Cmaxnorm: Cmax divided by the administered dose (in kg/L); Tmax: Time point at which Cmax was observed (in h). Parameters calculated from both, i.v. and i.g. concentration-time profiles: AUCnorm: Area under the concentration-time curve from t=0 h to infinity (extrapolated) divided by the administered dose (in kg*h/L); AUC(0−tlast)norm: Area under the concentration-time curve from t=0 h to the last time point for which plasma concentrations could be measured divided by the administered dose (in kg*h/L); t½: terminal half-life (in h); F: oral bioavailability: AUCnorm after intragastral administration divided by AUCnorm after intravenous administration (in %).

TABLE 3
In vivo PK in male rats at 2 mg/kg (p.o).

| Example | t_{1/2} [h] | F [%] |
|---|---|---|
| 1.1 | 5.2 | 32 |
| 3.1 | 17 | 87 |
| 8.1 | 5.0 | 17 |
| 10.1 | 3.3 | 23 |

### 2.5. LH Suppression in the Ovariectomized Rat

The in vivo potency of GnRH antagonists can be quantified by a LH suppression test in ovariectomized rats. GnRH triggers the LH release from the pituitary mediated by GnRH receptors. Ovarectomy of adult female rats results in elevated levels of circulating LH due to a lack of negative feedback by gonadal steroids. GnRH antagonists suppress the release of LH and accordingly suppression of LH levels can be used to quantify the in vivo potency of GnRH antagonists.

Female adult rats were ovariectomized surgically and they were allowed to recover for at least one week. The animals received 0.5 mg/kg, 3 mg/kg or 10 mg/kg of Example 3.1 by single per oral administration. For comparison reasons a vehicle control and a positive control, 0.1 mg/kg cetrorelix (i.p.), were given once. At 0 min, 15 min, 30 min, 1 hour, 2 hours, 4 hours, 8 hours and 24 hours after compound administration blood was taken from the retro orbital plexus (n=6 per blood withdrawal) for the measurement of serum LH and serum compound levels.

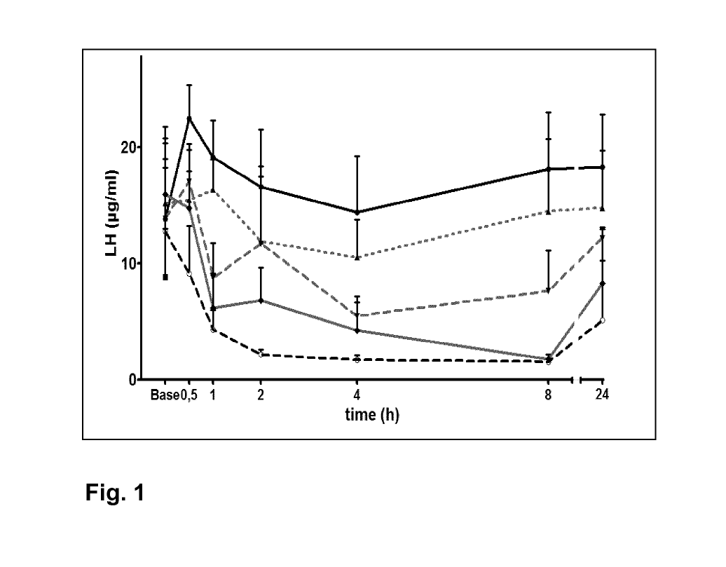

Per oral administration of Example 3.1 to ovariectomized rats resulted in a LH suppression of 19% (0.5 mg/kg), 48% (3 mg/kg) and 90% (10 mg/kg) at 8 hours following administration (see FIG. 1). Similarly, the positive control 0.1 mg/kg cetrorelix (i.p.) suppressed the LH level by ca. 90% at 8 hours.

To conclude Example 3.1 is an orally active GnRH antagonist in vivo.

### FIGURES

As nonbinding explanatory example of compounds according to the invention FIG. 1 represents the LH level following administration of the compound according to Example 3.1, to ovariectomized adult rats. [Filled circle: Vehicle; Circle, dotted black line: Cetrorelix (0.1 mg/kg); Triangle: Example 3.1 (0.5 mg/kg); Reverse triangle: Example 3.1 (3 mg/kg); Diamond: Example 3.1 (10 mg/kg)]. Values are given as mean+standard deviation (n=6).

1. A compound according to Formula (I) 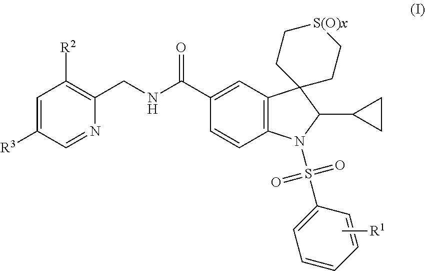
in which
x=0, 1 or 2;
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN;
with the proviso of N-[(3-chloro-5-fluoropyridin-2-yl)methyl]-2-cyclopropyl-1-[(4-fluorophenyl)sulfonyl]-1,2,2′,3′,5′,6′-hexahydrospiro [indole-3,4′-thiopyran]-5-carboxamide 1′,1′-dioxide.

2. The compound of claim 1, wherein 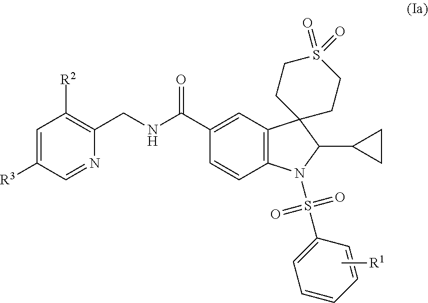
x is 2;
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN;
with the proviso of N-[(3-chloro-5-fluoropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide.

3. The A compound of Formula (I) of claim 1 wherein:
in which
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN.

4. The compound of claim 1, wherein
x is 0;
R¹ is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂;
R² is selected from the group consisting of halogen, hydroxy, C₁-C₄-alkyl, halo-C₁-C₄-alkyl, C₁-C₄-alkoxy, halo-C₁-C₄-alkoxy, CN;
R³ is selected from the group consisting of halogen, hydroxy, C₁-C₄-alkyl, halo-C₁-C₄-alkyl, C₁-C₄-alkoxy, halo-C₁-C₄-alkoxy, CN.

5. The compound of claim 1 characterized in that
R¹ is a single group in para or meta position and is selected from the group consisting of halogen, hydroxy, C₁-C₄-alkyl, halo-C₁-C₄-alkyl, C₁-C₄-alkoxy, halo-C₁-C₄-alkoxy, CN, C(O)NH₂.

6. The compound of claim 5 characterized in that
R¹ is a single group in para or meta position selected from the group consisting of halogen, C₁-C₄-alkoxy, halo-C₁-C₄-alkoxy, CN, C(O)NH₂.

7. The compound of claim 6 characterized in that
R¹ is a single group in para position selected from the group consisting of F, Cl, OCF₂H, CN, C(O)NH₂.

8. The compound of claim 6 characterized in that
R¹ is a single group in meta position selected from the group consisting of OCH₃, OCF₂H, OCF₃, CN.

9. The compound of claim 1 characterized in that
R² is selected from the group consisting of halogen, halo-C₁-C₄-alkyl.

10. The compound of claim 9 characterized in that
R² is selected from the group consisting of F, Cl, CF₃.

11. The compound of claim 1 characterized in that
R³ is selected from the group consisting of halogen, C₁-C₄-alkyl, halo-C₁-C₄-alkyl.

12. The compound of claim 11 characterized in that
R³ is selected from the group consisting of Cl, CH₃, CF₃.

13. The compound of the claim 1 characterized in that
R² is selected from the group consisting of F, Cl, CF₃, and
R³ is selected from the group consisting of Cl, CH₃, CF₃.

14. The compound of the claim 1 characterized in that
R² is selected from the group consisting of Cl, and
R³ is selected from the group consisting of CF₃.

15. The compound of claim 1, selected from the group consisting of
2-cyclopropyl-1-[(4-fluorophenyl)sulfonyl]-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide;
N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′-oxide;
N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide;
N-{[5-chloro-3-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide;
2-cyclopropyl-1-[(4-fluorophenyl)sulfonyl]-N-{[5-methyl-3-(trifluoromethyl)pyridin-2-yl]methyl}-1,2,2′,3′,5′,6′-hexahydrospiro[indole-3,4′-thiopyran]-5-carboxamide 1′,1′-dioxide;
2-cyclopropyl-N-{[3-fluoro-5-(trifluoromethyl)pyridin-2-yl]methyl}-1-[(3-methoxyphenyl)sulfonyl]-1,2,2′,3′,5′,6′-hexahydrospiro[indole-3,4′-thiopyran]-5-carboxamide 1′,1′-dioxide;
1-[(4-cyanophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide;
N-{[3-chloro-5-(trifluoromethyl)pyridin-2-yl]methyl}-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide;
1-[(3-cyanophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide;
N-{[3-chloro-5-(trifluoromethyl)pyridin-2-yl]methyl}-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide;
2-cyclopropyl-N-{[3-fluoro-5-(trifluoromethyl)pyridin-2-yl]methyl}-1-{[3-(trifluoromethoxy)phenyl]sulfonyl}-1,2,2′,3′,5′,6′-hexahydrospiro[indole-3,4′-thiopyran]-5-carboxamide 1′,1′-dioxide;
N-{[3-chloro-5-(trifluoromethyl)pyridin-2-yl]methyl}-2-cyclopropyl-1-{[3-(trifluoromethoxy)phenyl]sulfonyl}-1,2,2′,3′,5′,6′-hexahydrospiro[indole-3,4′-thiopyran]-5-carboxamide 1′,1′-dioxide;
2-cyclopropyl-1-{[3-(difluoromethoxy)phenyl]sulfonyl}-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide;
N-{[3-chloro-5-(trifluoromethyl)pyridin-2-yl]methyl}-2-cyclopropyl-1-{[3-(difluoromethoxy)phenyl]sulfonyl}-1,2,2′,3′,5′,6′-hexahydrospiro[indole-3,4′-thiopyran]-5-carboxamide 1′,1′-dioxide;
2-cyclopropyl-1-{[4-(difluoromethoxy)phenyl]sulfonyl}-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide;
N-{[3-chloro-5-(trifluoromethyl)pyridin-2-yl]methyl}-2-cyclopropyl-1-{[4-(difluoromethoxy)phenyl]sulfonyl}-1,2,2′,3′,5′,6′-hexahydrospiro[indole-3,4′-thiopyran]-5-carboxamide 1′,1′-dioxide;
1-[(4-carbamoylphenyl)sulfonyl]-N-{[3-chloro-5-(trifluoromethyl)pyridin-2-yl]methyl}-2-cyclopropyl-1,2,2′,3′,5′,6′-hexahydro spiro[indole-3,4′-thiopyran]-5-carboxamide 1′,1′-dioxide;
1-[(4-chlorophenyl)sulfonyl]-2-cyclopropyl-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide;
1-[(4-chlorophenyl)sulfonyl]-N-{[3-chloro-5-(trifluoromethyl)pyridin-2-yl]methyl}-2-cyclopropyl-1,2,2′,3′,5′,6′-hexahydro spiro[indole-3,4′-thiopyran]-5-carboxamide 1′,1′-dioxide; and
N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro[indole-3,4′-thiopyran]-5-carboxamide.

16. The compound of claim 1, characterized in that
the chiral configuration for the carbon atom in position 2 of the 1,2,2′,3′,5′,6′-hexahydrospiro[indole-3,4′-thiopyran] ring is S.

17. The compound of claim 1 selected from the group consisting of:
(2S)-2-cyclopropyl-1-[(4-fluorophenyl)sulfonyl]-N-{[3-fluoro-5-(trifluoromethyl)pyridin-2-yl]methyl}-1,2,2′,3′,5′,6′-hexahydrospiro[indole-3,4′-thiopyran]-5-carboxamide 1′,1′-dioxide;
(2S)—N-{[3-chloro-5-(trifluoromethyl)pyridin-2-yl]methyl}-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide; and
(2S)—N-{[3-chloro-5-(trifluoromethyl)pyridin-2-yl]methyl}-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide.

18. The compound of claim 1, wherein the compound is
(2S)—N-{[3-chloro-5-(trifluoromethyl)pyridin-2-yl]methyl}-2-cyclopropyl-1-[(4-fluorophenyl)sulfonyl]-1,2,2′,3′,5′,6′-hexahydrospiro [indole-3,4′-thiopyran]-5-carboxamide 1′,1′-dioxide.

19. (canceled)

20. A method of treatment of endometriosis, uterine leiomyoma (fibroids), polycystic ovarian disease, menorrhagia, dysmenorrhea, hirsutism, precocious puberty, gonadal steroid-dependent neoplasia, cancers of the prostate, breast and ovary, gonadotrope pituitary adenomas, sleep apnea, irritable bowel syndrome, premenstrual syndrome, benign prostatic hypertrophy, infertility, assisted reproductive therapy, in the treatment of growth hormone deficiency and short stature, and in the treatment of systemic lupus erythematosus comprising administering to a patient an effective amount of a compound of claim 1.

21. A method of contraception comprising administering an effective amount of a compound of claim 1 to a human female.

22. A pharmaceutical composition comprising a compound of claim 1 and a pharmaceutically acceptable carrier.

23. A chemical intermediate selected from the group consisting of:
5-bromo-1-[(4-chlorophenyl)sulfonyl]-2-cyclopropyl-1,2,2′,3′,5′,6′-hexahydrospiro[indole-3,4′-thiopyran];
5-bromo-1-[(4-chlorophenyl)sulfonyl]-2-cyclopropyl-1,2,2′,3′,5′,6′-hexahydrospiro[indole-3,4′-thiopyran] 1′,1′-dioxide;
methyl 2-cyclopropyl-1-[(4-fluorophenyl)sulfonyl]-1,2,2′,3′,5′,6′-hexahydrospiro[indole-3,4′-thiopyran]-5-carboxylate;
methyl 2-cyclopropyl-1-[(4-fluorophenyl)sulfonyl]-1,2,2′,3′,5′,6′-hexahydrospiro[indole-3,4′-thiopyran]-5-carboxylate 1′-oxide;
methyl 1-[(4-chlorophenyl)sulfonyl]-2-cyclopropyl-1,2,2′,3′,5′,6′-hexahydrospiro[indole-3,4′-thiopyran]-5-carboxylate 1′,1′-dioxide;
2-cyclopropyl-1-[(4-fluorophenyl)sulfonyl]-1,2,2′,3′,5′,6′-hexahydrospiro[indole-3,4′-thiopyran]-5-carboxylic acid 1′-oxide; and
1-[(4-chlorophenyl)sulfonyl]-2-cyclopropyl-1,2,2′,3′,5′,6′-hexahydrospiro[indole-3,4′-thiopyran]-5-carboxylic acid 1′,1′-dioxide.

24. A chemical intermediate selected from the group consisting of:
(2S)-5-bromo-2-cyclopropyl-1-[(4-fluorophenyl)sulfonyl]-1,2,2′,3′,5′,6′-hexahydrospiro[indole-3,4′-thiopyran]; and
(2S)-5-bromo-2-cyclopropyl-1-[(4-fluorophenyl)sulfonyl]-1,2,2′,3′,5′,6′-hexahydrospiro[indole-3,4′-thiopyran] 1′,1′-dioxide.
